(19) United States
(12) Patent Application Publication
(10) Pub. No.: US 2025/0025353 A1
(43) Pub. Date: Jan. 23, 2025

(54) APPARATUS AND TREATMENT FOR WOUNDS AND MALODOROUS ODOR AND WOUNDS

(71) Applicant: Alberto HACES, Parkland, FL (US)

(72) Inventor: Alberto HACES, Parkland, FL (US)

(21) Appl. No.: 18/898,167

(22) Filed: Sep. 26, 2024

Related U.S. Application Data
(63) Continuation-in-part of application No. 17/985,823, filed on Nov. 12, 2022.
(63) Continuation-in-part of application No. 17/339,879, filed on Jun. 4, 2021.
(60) Provisional application No. 63/540,910, filed on Sep. 27, 2023.
(60) Provisional application No. 63/280,591, filed on Nov. 17, 2021.
(60) Provisional application No. 63/082,465, filed on Sep. 23, 2020.

(51) Int. Cl. A61F 13/511; A61F 13/00; A61F 13/512

(52) U.S. Cl. CPC A61F 13/51113; A61F 13/00063; A61F 13/512

(57) ABSTRACT

A wound treatment barrier assembly comprising a volume encapsulating body carrying a wound treatment composition carrier is secured to a patient's skin at a location encapsulating a wound on a body of the patient. An iodine composition is emitted from the carrier in a gaseous state. The wound treatment composition carrier is removably inserted into a composition carrier receiving aperture formed through the volume encapsulating body. The gaseous emission remains within an interior volume of the volume encapsulating body. The design of the volume encapsulating body can avoid any contact between the volume encapsulating body, the wound treatment composition carrier and the wound. The volume encapsulating body is preferably domed in shape. An attachment flange can circumscribe a peripheral edge of the volume encapsulating body. An absorbent material can be provided between the attachment flange and the patient's skin.

### CROSS-REFERENCE TO RELATED APPLICATION

This Non-Provisional Utility patent application is:
A) a Non-Provisional patent application claiming the benefit of U.S. Provisional Patent Application Ser. No. 63/540,910 filed on Sep. 27, 2023;
B) a Continuation-In-Part (CIP) claiming the benefit of U.S. Non-Provisional patent application Ser. No. 17/985,823 filed on Nov. 12, 2022,
   wherein Non-Provisional patent application Ser. No. 17/985,823 claims the benefit of U.S. Provisional Patent Application 63/280,591 filed on Nov. 12, 2021;
C) a Continuation-In-Part (CIP) claiming the benefit of U.S. Non-Provisional patent application Ser. No. 17/339,879 filed on Jun. 4, 2021,
   wherein Non-Provisional patent application Ser. No. 17/339,879 claims the benefit of U.S. Provisional Patent Application Ser. No. 63/028,465 filed on Sep. 23, 2020;
   wherein each of the above identified applications are incorporated herein in their entireties by reference.

### FIELD OF THE INVENTION

The present invention relates generally to the field of wound treatment and more specifically relates to an apparatus and method of use of applying a wound treatment composition in a gaseous state to a wound using a barrier retaining the wound treatment composition within an interior volume thereof. The wound barrier and wound composition delivery device remain distant and contact free from the wound.

### BACKGROUND OF THE PRESENT INVENTION

Chronic skin wounds affect an estimated 6.5 million people in the United States, with treatment costs accounting for up to $25 billion per year. Populations at risk for chronic wounds include the elderly and those with co-morbidities such as diabetes and obesity. If not treated properly, chronic wounds can lead to severe local infection, sepsis, tissue or limb amputation, or death.

Treatment of dermatological wounds has generally been accomplished using a topical application, an oral application, an injected application, or any combination thereof. Topical applications are usually at least one of iodine, iodine releasing compounds (Iodophores), and other antimicrobial compounds.

The topical applications are customarily applied directly to the epidermis, or the outermost layer of skin. The topical applications are applied using an applicator, such as a cotton swab and either left exposed, or more commonly covered using a sterile covering such as a gauze pad, a pad in combination with an adhesive tape, an adhesive bandage, or other suitable covering.

The topical treatment can be applied in a form of a medicated adhesive patch. The medicated adhesive patch is an adhesive pad comprising a pre-applied volume of the topical treatment composition. Medicated adhesive patches can be applied to the patient's skin for surface treatment, such as injuries to the skin or transdermal treatment for delivery of a dosage of medication through the skin into the bloodstream.

Iodine, as tincture, or as iodine releasing compounds (Iodophores), and other antimicrobial compounds have been used topically for many years in the treatment of wounds (Sibbald R G, Leaoer D J, Queen D. Iodine Made Easy. Wounds International 2011; 2(2): Source: http://www.woundsintemational.com). These topically applied compositions are mostly applied to prevent wound or skin infections. If topical antimicrobials are used as soon as a wound is created, antibiotics might not be necessary. Most of these germicides are applied using a vehicle, such in water solution, in a hydrogel, or in some solid substrate used as dressing. Among the iodophores, povidone iodine solution (PVP-I) and cadexomer iodine are the most frequently used. However, the PVP-I can be toxic at the amounts used or, can irritate the wound. In a case of a cadexomer iodine (an iodine/polysaccharide gel complex, such as IODOFLEX® and IODOSORB® used as antiseptic fillers in cavity wounds), this treatment protocol requires continuous replacement of the dressings, which is time consuming, requires some professional training, and is not always effective. Traditional wound dressings stick to the wound and surrounding skin, where, at times can aggravate or re-injure the wound and/or the area surrounding the wound during removal of the wound dressing from the skin of the patient.

For many years, the wound care paradigm was to leave the cleaned and disinfected wound exposed to the air (dry healing) for a faster and less infective healing before applying sutures (In Flanders fields: the Great War, Antoine Depage, and the resurgence of débridement. Helling T S, Daon E Ann Surg. 1998 August; 228(2):173-81). It is known that oxygen plays a role in many of the stages of healing (Gottrup F. Oxygen in wound healing and infection. World J Surg, 2004; 28(3):312-315. doi:10.1007/s00268-003-7398-5, Gallagher K A, Liu Z J, Xiao M, et al.) Diabetic impairments in NO-mediated endothelial progenitor cell mobilization and homing are reversed by hyperoxia and SDF-1 alpha. (J Clin Invest. 2007; 117(5):1249-1259. doi:10.1172/JCI29710), while hypoxia is only needed at the beginning of the healing process (Demidova-Rice T N, Hamblin M R, Herman I M, Acute and impaired wound healing: pathophysiology and current methods for drug delivery, part 1: normal and chronic wounds: biology, causes, and approaches to care, Adv Skin Wound Care. 2012; 25(7):304-314, doi:10.1097/01.ASW.0000416006.55218.d0). But since the work of Winter (Formation of the scab and the rate of epithelization of superficial wounds in the skin of the young domestic pig. WINIER GD, Nature. 1962; 193:293-4), the wound-care community now believes that moist wounds heal faster and without a scab. To effect their healing properties, moist dressings are periodically applied to the wound several times a day to protect it from infection, but the moist dressings also prevent necessary oxygen from permeating into the wound. Further, the removal of the moist dressings can aggravate or re-injure the wound and/or the area surrounding the wound as well. Moist dressings deter production of a scab and lead to less scar tissue, but require more manipulations than a scab-forming, natural healing process.

While healing more slowly, a non-infected dry scab, being a natural dressing, will protect the underlying tissue from further infection if not disturbed, eliminating the need of dressing re-application while letting nature take its course. Most mothers in their wisdom, tell their children not to pick on their wound scabs; they are right about this (The molecular biology of wound healing: The Molecular Biology of Wound Healing. PLoS Biol. 2004; 2(8):e278. doi:10.1371/journal.pbio.0020278, Galko M J, Krasnow M A.)(Cellular and genetic analysis of wound healing in Drosophila larvae. PLoS Biol. 2004; 2(8):E239. doi:10.1371/journal.pbio.0020239)

The scab formation is greatly accelerated in a dry, oxygen rich environment. In fact, scab formation is an early sign of healing, but not all wounds form scabs and many become ulcers or non-healing wounds. Sometimes an ulcer can form without an abrasive force, just enough pressure in the foot or other body part (Bhattacharya S, Mishra R K. Pressure ulcers: Current understanding and newer modalities of treatment. Indian J Plast Surg. 2015; 48(1):4-16. doi:10.4103/0970-0358.155260) can form a pressure ulcer (PU). Therefore, an accelerated scab formation and healing would be a desirable quality in an antimicrobial agent to dry PUs. To date, the only suitable agent with such desirable properties is nitric oxide (NO), a natural bactericidal and healing promoter gas produced by endothelial cells. But delivery of nitric oxide (NO) is difficult due to a resulting toxicity, as the nitric oxide (NO) reacts with oxygen to produce toxic nitrogen dioxide (Schairer D O, Chouake J S, Nosanchuk J D, Friedman A J. The potential of nitric oxide releasing therapies as antimicrobial agents, Virulence. 2012; 3(3):271-279. doi:10.4161/viru.20328).

Typically, the wound treatment apparatus must be removed in order to change any medication.

Another common issue that needs to be addressed when treating patients is seepage/drainage from the wound. The seepage/drainage is typically problematic for both the patient and the medical staff. The seepage/drainage can cause discomfort to the patient, soil an area surrounding the wound, transfer to bedding, medical equipment, etc., can increase risks of infection, transfer/expose other parts of the patient and the medical staff to the biohazardous materials, and many other undesirable issues. The seepage/drainage is typically only partially retained by the dressing with excessive seepage/drainage passing through the dressing. This increases a frequency of changing of dressings.

Wound dressings are typically opaque. Inspection of the wound requires removal of the dressing. Removal of the dressing introduces a number of concerns, such as requirement of an application of a new dressing, exposure to potential contamination, removal of the dressing comprising an adhesive can aggravate or reinjure the wound increasing healing time, and many other adverse affects.

What is desired is an apparatus and a method of treatment for aiding in recovery of one or more wounds. Additionally, it is desired that the wound treatment apparatus can remain in place when changing the medication. It is further desired that the wound treatment apparatus optimize retention of any seepage/drainage from the wound. It is also advantageous to provide a wound dressing that enables inspection of the wound without removal of the wound dressing.

Different bacteria and/or fungi commonly invade a person's body creating a bacterial infection or a fungal infection. Bacteria and fungi congregate in moist areas on the body, such as feet and underarms. In certain instances the bacteria invades wounds, creating an infection. A majority of bacteria and fungi emits a malodorous odor. Athlete's foot is one example of a fungal infection. Athlete's foot (tinea pedis) is a fungal skin infection that usually begins between the toes. Symptoms include a scaly rash that usually causes itching, stinging, and burning. People with athlete's foot commonly have moist, raw skin between their toes. Currently recommended treatment involves topical antifungal medications. It commonly occurs in people whose feet have become very sweaty while confined within tight-fitting shoes. Another method of addressing foot odor is use of an insole impregnated with charcoal. The charcoal absorbs the malodorous odor emitted from the foot and remaining in a shoe. Charcoal addresses the odor, but fails to address the root cause of the odor.

What is desired is an apparatus and a method of treatment to kill the bacteria and/or fungi and other odor generating organisms, resulting in an elimination of the malodorous odor.

### SUMMARY OF THE PRESENT INVENTION

Recently, the Inventor has discovered that elemental iodine gas or vapor dramatically accelerates the drying as well as the scab formation process in wounds. This is an aerobic, novel, mild, controllable, non-contact delivery of antimicrobials or therapeutics. The inventive delivery can be stopped at any time, whereas PV-I, or other topical agents, cannot be removed once applied. The latter also need a dressing to cover the wound. Thus, iodine gas should be considered more efficacious than PV-I with much less toxicity. Additionally, oral elemental iodine, in vitro and on pre-clinical trials, shows anti-proliferative effects in benign and malignant neoplasia such as benign prostate hyperplasia (BPH) and breast and prostate cancers (Aceves, C., Anguiano, B., Delgado, G: The Extrathyronine Actions of Iodine as Antioxidant, Apoptotic, and Differentiation Factor in Various Tissues, Thyroid, Volume 23, Number 8, 2013. https://doi.org/10.1089/thy.2012.0579). Furthermore, the present invention can be adapted to deliver NO in a simpler, contactless way as well. Currently, known nitric oxide (NO) delivery methods utilize either a contact wound dressing; a patch that releases the gas from a chemical reaction; or using direct gas from a gas bottle. Additionally, the method of delivery provide by the present invention can be employed to deliver any volatile antimicrobial agents (Swamy M K, Akhtar M S, Sinniah U R. Antimicrobial Properties of Plant Essential Oils against Human Pathogens and Their Mode of Action: An Updated Review. Evid Based Complement Alternat Med. 2016; 2016:3012462. doi:10.1175/2016/3012462) to wounds such as: essential oils and/or their active components, oxygen or any other volatile therapeutics.

The invention provides an apparatus and a respective method to overcome the main deficiencies of the current state of the art by providing a novel method of delivering antimicrobials to wounds in a gentler, more effective method. The method of the present invention provides a wound-contactless gas chamber created by a contactless wound treatment delivery body in contact with skin surrounding a wound of a patient. The gas chamber positioned above the wound is filled with at least one of (a) an antimicrobial gas and (b) a healing gas. The gas chamber can be secured in position over the wound using any suitable attachment element, including adhesive, double sided adhesive, an elastic band, a rubber band, a ribbon, a strap, a bandage, and the like.

In a first aspect of the present invention, a contactless wound treatment barrier assembly includes:
a wound treatment barrier body formed to provide an interior volume defined by an interior surface of the wound treatment barrier body extending from an attachment peripheral edge forming an enclosed space,
a wound treatment composition carrier assembled to the interior surface of the wound treatment barrier body, and
a wound treatment composition applied to the wound treatment composition carrier.

In a second aspect, the wound treatment barrier body can be fabricated of a breathable material.

In another aspect, the wound treatment barrier body can be fabricated of a non-breathable material.

In another aspect, the wound treatment barrier body can be fabricated of Ethylene-Vinyl Acetate Copolymer (EVA).

In another aspect, the wound treatment barrier body can be fabricated of Poly Ethylene-Vinyl Acetate Copolymer (PEVA).

In another aspect, the wound treatment barrier body can be shaped to include an interior volume defining a chamber having a size to confine the gas for enough time to produce a desired biological effect during application of the wound treatment composition in a gaseous state.

In yet another aspect, the contactless wound treatment barrier assembly further comprising a contact flange extending radially outward from the attachment peripheral edge of the wound treatment barrier body.

In another aspect, the wound treatment barrier body can have a domed shape.

In another aspect, the wound treatment barrier body can have a domed shape including a flange extending generally radially outward from a peripheral edge of the dome.

In another aspect, the wound treatment barrier body can have a domed shape including a flange extending generally radially outward from a peripheral edge of the dome, the wound treatment barrier body further comprising an adhesive secured to an attachment side of the flange.

In another aspect, the wound treatment barrier body further comprising an aperture.

In another aspect, the wound treatment barrier body further comprising an aperture passing therethrough.

In another aspect, the wound treatment barrier body having a domed shape and an aperture passing through an upper portion of the dome.

In another aspect, the wound treatment barrier body having a domed shape and an aperture passing through a central upper portion of the dome.

In another aspect, the wound treatment barrier body can have a shape for a foot covering.

In another aspect, the wound treatment barrier body can have a shape for a foot covering and an aperture passing through an upper portion of the foot covering.

In another aspect, the wound treatment barrier body can have a shape for a foot covering and at least one aperture passing through an upper portion of the foot covering.

In another aspect, the wound treatment barrier body can have a shape for a foot covering and a plurality of apertures passing through an upper portion of the foot covering.

In another aspect, the wound treatment barrier body can have a shape for a foot covering and a plurality of apertures passing through an upper portion of the foot covering, wherein at least a portion of the apertures are sealed and at least one wound treatment composition storage and delivery container assembly is assembled therethrough.

In another aspect, the wound treatment barrier body can have a shape for a foot covering and a plurality of apertures passing through an upper portion of the foot covering, wherein at least a portion of the apertures are sealed with a transparent material and at least one wound treatment composition storage and delivery container assembly is assembled therethrough.

In another aspect, the wound treatment barrier body can have a shape for a foot covering and a plurality of apertures passing through an upper portion of the foot covering, wherein at least a portion of the apertures are used to deliver a gas into an interior of the wound treatment barrier body and at least one wound treatment composition storage and delivery container assembly is assembled therethrough.

In another aspect, the wound treatment barrier body can have a shape for a foot covering and a plurality of apertures passing through an upper portion of the foot covering, wherein at least a portion of the apertures are used to deliver a gas into an interior of the wound treatment barrier body and at least one wound treatment composition storage and delivery container assembly is assembled therethrough, wherein the gas maintains a spatial relation between the wound and the wound treatment composition within the wound treatment composition storage and delivery container assembly.

In another aspect, the wound treatment barrier body can have a shape for a foot covering and a plurality of apertures passing through an upper portion of the foot covering, wherein at least one aperture of the plurality of apertures includes a clear material enabling viewing therethrough.

In another aspect, the wound treatment barrier body can have a shape for a foot covering and a plurality of apertures passing through an upper portion of the foot covering, wherein at least one aperture of the plurality of apertures includes a removable clear material enabling viewing therethrough.

In another aspect, the wound treatment barrier body can have a shape for a foot covering, wherein the foot covering is fabricated of a gas impervious material.

In another aspect, the wound treatment barrier body can have a shape for a foot covering, the foot covering comprising a J-shaped covering.

In another aspect, the wound treatment barrier body can have a shape for a foot covering, the foot covering comprising a J-shaped bag.

In another aspect, the wound treatment barrier body can have a shape for a foot covering, the foot covering comprising a J-shaped covering, wherein the wound treatment barrier body is fabricated of a gas impervious material.

In another aspect, the wound treatment barrier body can have a shape for a foot covering, the foot covering comprising a J-shaped bag, wherein the wound treatment barrier body is fabricated of a gas impervious material.

In another aspect, the wound treatment barrier body can have a shape for a foot covering, the foot covering comprising an upper attached to a sole.

In another aspect, the wound treatment barrier body can have a shape for a foot covering, the foot covering comprising an upper attached to a sole, wherein the sole is fabricated of a semi-rigid material.

In another aspect, the wound treatment barrier body can have a shape for a foot covering, the foot covering comprising an upper attached to a sole, wherein the sole is fabricated of a semi-rigid material such as a foam, rubber, nylon, flexible plastic, and the like.

In another aspect, the wound treatment barrier body can have a shape for a foot covering, the foot covering comprising an upper attached to a sole, wherein the upper is fabricated of a flexible gas retaining material.

In another aspect, the wound treatment barrier body can have a shape for a foot covering, the foot covering comprising an upper attached to a sole, wherein the upper is fabricated of a flexible gas impervious material.

In another aspect, the wound treatment barrier body can have a shape for a foot covering, the foot covering comprising an upper attached to a sole, wherein the upper is fabricated of a semi-rigid material such as a foam, rubber, silicone, silicone-rubber, nylon, flexible plastic, clear flexible plastic, leather, and vinyl, and the like.

In another aspect, the wound treatment barrier body can have a shape for a foot covering, the foot covering comprising an upper attached to a sole, wherein the sole is fabricated of a semi-rigid material such as a foam, rubber, nylon, flexible plastic, and the like.

In another aspect, the wound treatment barrier body can have a shape for a foot covering, the foot covering comprising an upper attached to a sole, the foot covering providing a substantial gas seal between the foot covering and a patient's lower leg.

In another aspect, the wound treatment barrier body can have a shape for a foot covering, the foot covering comprising an upper attached to a sole, a banding about an upper portion of the foot covering providing a substantial gas seal between the foot covering and a patient's lower leg.

In another aspect, the wound treatment barrier body can have a shape for a foot covering, the foot covering comprising an upper attached to a sole, an elastic sealing flange extending radially across the upper portion of the foot covering, the elastic sealing flange comprising an aperture for passing a foot and leg of the patient therethrough, the elastic sealing flange providing a substantially gas impervious seal between the foot covering and lower portion of a patient's leg.

In another aspect, wherein the elastic sealing flange is alternatively referred to as a diaphragm seal.

In another aspect, wherein the elastic sealing flange is fabricated of a silicone material.

In another aspect, wherein the elastic sealing flange is fabricated of a medical-grade silicone material.

In another aspect, wherein the elastic sealing flange is fabricated of a silicone-rubber material.

In another aspect, wherein the elastic sealing flange is fabricated of a rubber material.

In another aspect, wherein the elastic sealing flange is fabricated of a latex free material.

In another aspect, the wound treatment barrier body further comprising an aperture, wherein the aperture is sized to receive the wound treatment composition carrier.

In another aspect, the wound treatment composition carrier is inserted through the aperture of the wound treatment barrier body.

In another aspect, the wound treatment composition carrier is threadably assembled into the aperture of the wound treatment barrier body.

In another aspect, the wound treatment composition carrier is assembled into a receiver inserted into the aperture of the wound treatment barrier body.

In another aspect, the wound treatment composition carrier is threadably assembled into a threaded receiver inserted into the aperture of the wound treatment barrier body.

In another aspect, the wound treatment barrier body further comprising a wound viewing aperture.

In another aspect, the wound treatment barrier body further comprising a wound viewing aperture, the wound viewing aperture being formed through the material of the wound treatment barrier body.

In another aspect, the wound treatment barrier body further comprising a wound viewing aperture, the wound viewing aperture being formed through the material of a domed portion of the wound treatment barrier body.

In another aspect, the wound treatment barrier body further comprising a wound viewing aperture, the wound viewing aperture being sealed by a clear material.

In another aspect, the contactless wound treatment barrier assembly further comprising heat generating element.

In another aspect, the heat generating element being located within the wound treatment barrier body.

In another aspect, the wound treatment composition carrier further comprising a mechanism to adjust a rate of dispensing of dispensed medication.

In another aspect, the wound treatment composition carrier further comprising a mechanism to adjust a rate of dispensing of dispensed medication, wherein the mechanism is programmable.

In another aspect, the wound treatment composition carrier further comprising a mechanism to adjust a rate of dispensing of dispensed medication, wherein the mechanism is programmable enabling programmable dispending of the medication.

In another aspect, the wound treatment composition carrier further comprising a mechanism to adjust a rate of dispensing of dispensed medication, wherein the mechanism is programmable enabling programmable dispending of the medication, wherein the programming is provided via a software instruction set.

In another aspect, the wound treatment composition carrier further comprising a mechanism to adjust a rate of dispensing of dispensed medication, wherein the mechanism is programmable enabling programmable dispending of the medication, wherein the programming is provided via a software instruction set, wherein the instruction set includes a time and volume for dispensing the medication.

In another aspect, the wound treatment composition carrier further comprising a mechanism to adjust a rate of dispensing of dispensed medication, wherein the mechanism is programmable enabling programmable dispending of the medication, wherein the programming is provided via a software instruction set, wherein the instruction set includes time periods and a volume for dispensing the medication associated with each time period.

In another aspect, the wound treatment composition carrier further comprising a mechanism to adjust a rate of dispensing of dispensed medication, wherein the mechanism is programmable enabling programmable dispending of the medication, wherein the programming is provided via a software instruction set provided by a medical professional.

In another aspect, the wound treatment composition carrier further comprising a mechanism to adjust a rate of dispensing of dispensed medication, wherein the mechanism is programmable enabling programmable dispending of the medication, wherein the programming is provided via a software instruction set provided by a medical professional via an internet.

In another aspect, the wound treatment composition carrier further comprising a mechanism to adjust a rate of dispensing of dispensed medication, wherein the rate adjusting mechanism rotates a member to adjust a dispensing opening in the wound treatment composition carrier.

In another aspect, the wound treatment barrier body further comprising a mechanism to nebulizer the dispensed medication.

In another aspect, the wound treatment barrier body further comprising a mechanism to circulate the dispensed medication.

In another aspect, the wound treatment barrier body further comprising a mechanism to circulate the dispensed medication, wherein the circulation mechanism is a fan.

In yet another aspect, the contactless wound treatment barrier assembly further comprising a contact flange extending radially outward from and circumscribing the attachment peripheral edge of the wound treatment barrier body.

In yet another aspect, the wound treatment composition is at least one of an antimicrobial compound, iodine, elemental iodine, and an iodine releasing compound.

In yet another aspect, the wound treatment composition is an antimicrobial compound.

In yet another aspect, the wound treatment composition is iodine.

In yet another aspect, the wound treatment composition is an iodine releasing compound.

In yet another aspect, the wound treatment composition is at least one of:
elemental iodine,
iodoform,
ethanol,
isopropyl alcohol,
maltol,
3-(phenylamino) butan-2-one (schleiferon A),
3-(phenylimino) butan-2-one (schleiferon B),
acetoin and 2-phenylethylamine,
elemental sulfur,
nonanol,
decanol,
undecanol,
dodecanol,
NO gas releasing compounds such as S-Nitrosoglutathione,
1-hydroxy-2-oxo-3, 3-bis(2-aminoethyl)1-triazene,
1-hydroxy-2-oxo-3, 3-bis(3-aminoethyl)1-triazene,
1-hydroxy-2-oxo-3-(3-aminopropyl) 3-(4-aminobutyl) 1-triazene
Oxygen,
essential antimicrobial oils from oregano,
thyme,
clove,
lavender,
clary sage,
arborvitae,
active oils eucalyptol,
thymol,
menthol,
carvacrol, thymol,
cinnamic aldehyde,
eugenol,
p-cymene,
allicine,
calendula oil,
melaleuca oil, and
lemongrass oil.

In yet another aspect, the wound treatment composition carrier impregnated with the wound treatment composition is stored within a moisture impervious container until ready for use.

In yet another aspect, the wound treatment composition is iodine impregnated activated charcoal embedded in the wound treatment composition carrier or inside of a pouch inside the wound treatment composition carrier.

In yet another aspect, the wound treatment composition carrier can be foam or silicone rubber.

In yet another aspect, the wound treatment composition carrier impregnated with the wound treatment composition is placed directly against the interior surface of the wound treatment barrier body and secured to the interior surface of the wound treatment barrier body using any suitable attachment configuration.

In yet another aspect, the wound treatment composition carrier impregnated with the wound treatment composition is placed directly against the interior surface of the wound treatment barrier body and secured to the interior surface of the wound treatment barrier body using a mechanical fastener.

In yet another aspect, the wound treatment composition carrier impregnated with the wound treatment composition is placed directly against the interior surface of the wound treatment barrier body and secured to the interior surface of the wound treatment barrier body using a mechanical fastener, wherein the mechanical fastener is a selected from a group of mechanical fasteners, the group of mechanical fasteners including a staple, a rivet, a snap stud and socket, a hook and loop, a dense hook and loop tape, an adhesive a double sided adhesive, a bonding agent, and the like.

In yet another aspect, the wound treatment composition carrier impregnated with the wound treatment composition is placed directly against the interior surface of the wound treatment barrier body and secured to the interior surface of the wound treatment barrier body using a magnetic attraction, wherein the magnetic attraction is provided by a magnetized element and a magnetically attracted element, such as an element fabricated of or containing a ferrous material. One of the magnetized element or the magnetically attracted element is secured to the wound treatment composition carrier; the other of the magnetized element or the magnetically attracted element is secured to the interior surface of the wound treatment barrier body at a desired location for delivery of the wound treatment composition.

In yet another aspect, the storage and delivery container body is sealed by a removable element.

In yet another aspect, the storage and delivery container body is sealed by a removable cap.

In yet another aspect, the storage and delivery container body is sealed by a removable foil.

In yet another aspect, the storage and delivery container body includes a tubular sidewall and an end wall comprising an attachment surface.

In yet another aspect, the storage and delivery container body includes the tubular sidewall and the end wall comprising an attachment surface, wherein interior surfaces of the tubular sidewall and end wall define an interior volume.

In yet another aspect, the storage and delivery container body includes a cylindrical sidewall and an end wall comprising an attachment surface.

In yet another aspect, the storage and delivery container body includes the cylindrical sidewall and the end wall comprising an attachment surface, wherein interior surfaces of the cylindrical sidewall and end wall define an interior volume.

In yet another aspect, the storage and delivery container body can further comprise a perforated end wall located proximate to an edge sealed by a removable element.

In yet another aspect, the storage and delivery container body comprising the wound treatment composition carrier impregnated with the wound treatment composition is placed against the interior surface of the wound treatment barrier body and secured to the interior surface of the wound treatment barrier body using a mechanical fastener, wherein the mechanical fastener is a selected from a group of mechanical fasteners, the group of mechanical fasteners including a staple, a rivet, a snap stud and socket, a hook and loop, a dense hook and loop tape, an adhesive a double sided adhesive, a bonding agent, and the like.

In yet another aspect, the storage and delivery container body comprising the wound treatment composition carrier impregnated with the wound treatment composition is placed against the interior surface of the wound treatment barrier body and secured to the interior surface of the wound treatment barrier body using a magnetic attraction, wherein the magnetic attraction is provided by a magnetized element and a magnetically attracted element, such as an element fabricated of or containing a ferrous material. One of the magnetized element or the magnetically attracted element is secured to the storage and delivery container body; the other of the magnetized element or the magnetically attracted element is secured to the interior surface of the wound treatment barrier body at a desired location for delivery of the wound treatment composition.

In yet another aspect, an adhesive is applied to a contacting surface of the contact flange.

In yet another aspect, a protective appliqué is applied to the adhesive on the contacting surface of the contact flange.

In yet another aspect, each end of an elastic band is secured to the contact flange of the contactless wound treatment barrier assembly.

In yet another aspect, a pair of elastic bands is secured to the contact flange of the contactless wound treatment barrier assembly, wherein each end of each elastic band of the pair of elastic bands is secured to the contact flange of the contactless wound treatment barrier assembly.

In yet another aspect, at least two elastic bands are secured to the contact flange of the contactless wound treatment barrier assembly, wherein each end of each elastic band of the at least two elastic bands is secured to the contact flange of the contactless wound treatment barrier assembly.

In yet another aspect, each end of a ribbon or other tie is secured to the contact flange of the contactless wound treatment barrier assembly.

In yet another aspect, a pair of ribbons or other ties is secured to the contact flange of the contactless wound treatment barrier assembly, wherein each end of each ribbon or other tie of the pair of ribbons or other ties is secured to the contact flange of the contactless wound treatment barrier assembly.

In yet another aspect, at least two ribbons or other ties are secured to the contact flange of the contactless wound treatment barrier assembly, wherein each end of each ribbon or other tie of the at least two ribbons or other ties is secured to the contact flange of the contactless wound treatment barrier assembly.

In yet another aspect, a method that treats a wound that is considered to be one of in a process of healing or non-healing in a way that their pathway to successful healing is initiated earlier in the wound healing process (i.e. formation of a scab) with minimal complications.

In yet another aspect, a therapeutically amount of constant anti-infective and healing agent is delivered to a wound providing minimal toxicity or exclusive of any toxicity.

In yet another aspect, a therapeutically amount of constant anti-infective and healing agent is delivered to a wound exclusive of any need for invasive/contact dressings.

In yet another aspect, the wound treatment compositions utilized to treat the wound are antimicrobial agents that can be delivered to the wound in the gaseous form by virtue of their natural gas state.

In yet another aspect, the wound treatment composition utilized to treat the wound is a wound treatment composition that can be delivered to the wound in the gaseous form, wherein the gaseous form of the wound treatment composition is generated from a wound treatment composition, the wound treatment composition being provided in one of a solid state or a liquid state by one of (a) easy sublimation or (b) the high volatility of the wound treatment composition at room temperature.

In yet another aspect, the wound treatment compositions utilized to treat the wound are antimicrobial agents that can be delivered to the wound in the gaseous form, wherein the gaseous form of the antimicrobial agents is generated from the antimicrobial agent, the antimicrobial agent being provided in one of a solid state or a liquid state by one of (a) easy sublimation or (b) the high volatility of the antimicrobial agent at room temperature.

In yet another aspect, the wound treatment compositions utilized to treat the wound are antimicrobial agents that can be delivered to the wound in the gaseous form by virtue of their natural gas state, or generated from a solid or liquid compound by easy sublimation or due to their high volatility at room temperature.

In yet another aspect, an apparatus enabling delivery of a therapeutic agent in a gaseous state to the wound without physical contact between the apparatus and the wound.

In yet another aspect, a method to deliver a therapeutic agent in the gaseous state to the wound without physical contact between the apparatus and the wound.

In yet another aspect, the apparatus includes a non-breathable, hermetic body structure to deliver oxygen sensitive antimicrobial and healing promoting gases to the wound.

In yet another aspect, the wound treatment composition carrier can be fabricated of one of a silicone rubber (with active component dissolved or “baked” in it), a porous foam, or any other substrate having properties that absorbs a liquid quickly and releases the absorbed liquid in a gaseous state at slower rate.

In yet another aspect, the apparatus include a void volume between an interior surface and a wound surface to allow the gas to fill that void and to avoid any contact of the device with the wound.

In yet another aspect, the wound treatment barrier body is fabricated of a partially breathable or non-breathable material that keeps the interior volume humid (and the wound moist to prevent from drying too quickly) by preventing any sweat or water evaporation from the skin to leave the interior volume (wound atmosphere) while saturating the healing atmosphere with the at least one of the gaseous antimicrobial and the healing agent.

In yet another aspect, the wound treatment barrier body creates a rigid chamber, wherein the rigidity of the chamber allows the patient to wear clothing and walk without a risk of the chamber collapsing onto the wound.

In yet another aspect, the chamber or vault where the delivery of the wound treatment composition or active compound is made by a pump or any other delivery device to the inner chamber.

In yet another aspect, the wound treatment barrier body provides a shallow chamber or vault with a minimum void space to deliver oxygen sensitive gases to minimize the amount of oxygen present in the inter-vault/wound void space.

In yet another aspect, the wound treatment composition can be coated upon the interior surface of the wound treatment composition storage and delivery container and covered with a seal until just before use.

In an alternative configuration, the wound treatment barrier body is formed within an insole designed for insertion within a shoe.

In one aspect, the insole is fabricated of a liquid absorbing material.

In a second aspect, the insole is fabricated of a liquid absorbing, or a gas absorbing, material, the liquid absorbing material being one of: a porous material, such as an open cell foam, a polyurethane, a reticulated polyurethane foam, a sponge; an absorbent material such as silicone rubber. The absorbing/releasing material can be infused with the liquid or a solid before the solidifying process during manufacturing said absorbing material.

In another aspect, the insole is fabricated of at least two insole bodies laminated to one another.

In another aspect, the insole comprises at least one receptacle for receiving and retaining a wound treatment composition carrier.

In another aspect, the insole comprises a plurality of receptacles for receiving and retaining a wound treatment composition carrier.

In another aspect, the insole comprises a plurality of receptacles for receiving and retaining a wound treatment composition carrier, wherein at least two receptacles are of a like shape and size.

In another aspect, a first insole body comprises a plurality of receptacles for receiving and retaining a wound treatment composition carrier, wherein at least two receptacles are of a like shape and size, while a second insole body comprises a plurality of apertures, each aperture located in registration with a respective receptacle.

In another aspect, the first insole body and the second insole body are fabricated of like materials.

In another aspect, the first insole body and the second insole body are fabricated of different materials.

In another aspect, a base or bottom insole body further comprising a peripheral edge dam.

In another aspect, the insole body is fabricated of a pliant material.

In another aspect, the insole body is fabricated of an absorbent material.

In another aspect, at least a portion of the insole body is fabricated of a microporous polyurethane foam.

In another aspect, a top surface of the insole body can be of a nylon material, a velvet material, a velour material, a polyester, Rayon, a satin material, or any other suitable material.

In another aspect, the insole body is fabricated of an absorbent material, the wound treatment composition being applied directly to the insole body.

In another aspect, the insole body further comprises a moisture impervious layer of material applied to at least one side of the insole body.

The present invention also describes a method of treating a wound, wherein the method of treatment comprising steps of:
securing a contactless wound treatment barrier assembly to a patient's skin, wherein the contactless wound treatment barrier assembly is located covering a wound to the patient's skin,
the contactless wound treatment barrier assembly comprising:
   a wound treatment barrier body formed to provide an interior volume defined by an interior surface of the wound treatment barrier body extending from an attachment peripheral edge forming an enclosed space,
   a wound treatment composition carrier assembled to the interior surface of the wound treatment barrier body,
   a wound treatment composition applied to the wound treatment composition carrier;
emitting the wound treatment composition in a gaseous state from the wound treatment composition carrier within the interior volume to treat the wound to the patient's skin.

In a second aspect, wherein the step of securing a contactless wound treatment barrier assembly to a patient's skin is accomplished using an adhesive.

In another aspect, wherein the step of securing a contactless wound treatment barrier assembly to a patient's skin is accomplished using an elastic band.

In yet another aspect, wherein the step of securing a contactless wound treatment barrier assembly to a patient's skin is accomplished using at least one elastic band.

In yet another aspect, wherein the step of securing a contactless wound treatment barrier assembly to a patient's skin is accomplished using a plurality of elastic bands.

In yet another aspect, wherein the step of securing a contactless wound treatment barrier assembly to a patient's skin is accomplished using a ribbon or tie.

In yet another aspect, wherein the step of securing a contactless wound treatment barrier assembly to a patient's skin is accomplished using at least one ribbon or tie.

In yet another aspect, wherein the step of securing a contactless wound treatment barrier assembly to a patient's skin is accomplished using a plurality of ribbons or ties.

In yet another aspect, wherein the step of securing a contactless wound treatment barrier assembly to a patient's skin is accomplished by inserting a treatment insole into an interior of a shoe.

In yet another aspect, the method of treating a wound further comprising a step of securing the wound treatment enclosure to the skin of a patient at a location proximate the wound in a manner where the wound treatment enclosure covers the wound.

In yet another aspect, the method of treating a wound further comprising a step of securing the wound treatment enclosure to the skin of a patient at a location proximate the wound in a manner where the wound treatment enclosure completely covers the wound.

In yet another aspect, the method of treating a wound further comprising a step of securing the wound treatment enclosure to the skin of a patient at a location proximate the wound in a manner where the wound treatment enclosure completely encases the wound.

In yet another aspect, the method of treating a wound further comprising a step of securing the wound treatment enclosure to the skin of a patient is accomplished using an adhesive provided on a contacting surface of the wound treatment enclosure.

In yet another aspect, the method of treating a wound further comprising a step of securing the wound treatment enclosure to the skin of a patient is accomplished using an adhesive provided on a contacting surface of a flange circumscribing the wound treatment enclosure.

In yet another aspect, the method of treating a wound further comprising a step of placing the wound treatment enclosure against the skin of a patient includes placement of an absorbent material between the wound treatment enclosure and the skin of the patient.

In yet another aspect, the method of treating a wound further comprising a step of placing the wound treatment enclosure against the skin of a patient includes placement of an absorbent material between the wound treatment enclosure and the skin of the patient, wherein the absorbent material follows a circumference of the body of the wound treatment enclosure.

In yet another aspect, the method of treating a wound further comprising a step of placing the wound treatment enclosure against the skin of a patient includes placement of an absorbent material between the wound treatment enclosure and the skin of the patient, wherein the absorbent material is bonded to the wound treatment enclosure.

In yet another aspect, the method of treating a wound further comprising a step of placing the wound treatment enclosure against the skin of a patient includes placement of an absorbent material between the wound treatment enclosure and the skin of the patient, wherein the absorbent material is bonded to a flange circumscribing the wound treatment enclosure.

In yet another aspect, the method of treating a wound further comprising a step of placing the wound treatment enclosure against the skin of a patient includes placement of an absorbent material between the wound treatment enclosure and the skin of the patient, wherein the absorbent material provides a seal between facing surfaces of the wound treatment enclosure and the skin of the patient.

In yet another aspect, the method of treating a wound further comprising a step of placing the wound treatment enclosure against the skin of a patient includes placement of an absorbent material between the wound treatment enclosure and the skin of the patient, wherein the absorbent material provides a complete seal between facing surfaces of the wound treatment enclosure and the skin of the patient.

In yet another aspect, the method of treating a wound further comprising a step of placing the wound treatment enclosure against the skin of a patient includes placement of an absorbent material between the wound treatment enclosure and the skin of the patient, wherein a first side of the absorbent material is bonded to the wound treatment enclosure and a second, opposite side is bonded to the skin of the patient.

In yet another aspect, the method of treating a wound further comprising a step of placing the wound treatment enclosure against the skin of a patient includes placement of an absorbent material between the wound treatment enclosure and the skin of the patient, wherein a first side of the absorbent material is bonded to the wound treatment enclosure and a second, opposite side is bonded to the skin of the patient, wherein at least one bonded interface employs a double sided adhesive.

In yet another aspect, the method of treating a wound further comprising a step of placing the wound treatment enclosure against the skin of a patient includes placement of an absorbent material between the wound treatment enclosure and the skin of the patient, wherein a first side of the absorbent material is bonded to the wound treatment enclosure and a second, opposite side is bonded to the skin of the patient, wherein both bonded interfaces employ the double sided adhesive.

In yet another aspect, the method of treating a wound further comprising a step of inserting the wound treatment composition carrier through an aperture formed through a body of the wound treatment enclosure.

In yet another aspect, the method of treating a wound further comprising a step of inserting the wound treatment composition carrier through an aperture formed through a body of the wound treatment enclosure, wherein the wound treatment composition carrier is threadably secured within the aperture.

In yet another aspect, the method of treating a wound further comprising a step of inserting the wound treatment composition carrier through an aperture formed through a body of the wound treatment enclosure, wherein the wound treatment composition carrier is threadably secured to a threaded receiver inserted within the aperture formed through a body of the wound treatment enclosure.

In yet another aspect, the method of treating a wound further comprising a step of inspecting the wound by viewing the wound through a viewing port formed through the body of the wound treatment enclosure.

In yet another aspect, the method of treating a wound further comprising a step of inspecting the wound by viewing the wound through a clear material provided with the viewing port formed through the body of the wound treatment enclosure.

In yet another aspect, the wound treatment applicator is fabricated of a material that absorbs the wound treatment composition and releases the wound treatment composition in a gaseous state.

In yet another aspect, the wound treatment applicator is fabricated of microporous polyurethane foam, wherein the microporous polyurethane foam absorbs the wound treatment composition and releases the wound treatment composition in a gaseous state.

In yet another aspect, the insole is fabricated of a plurality of layers laminated to one another in a stacked orientation.

In yet another aspect, the insole is fabricated of a plurality of layers laminated to one another in a stacked orientation, where a top layer is exclusive of any wound treatment composition.

In yet another aspect, the insole is fabricated of a plurality of layers laminated to one another in a stacked orientation, where the top layer is exclusive of any wound treatment composition and at least one lower layer is impregnated with the wound treatment composition.

In yet another aspect, the method can further comprise a step of wearing a sock on a foot being treated by an antimicrobial agent coated insole wherein the process relies on the patient's sock as a separating barrier. The antimicrobial agent can be applied to one or both sides of the coated insole.

In yet another aspect, the method can further comprise a step of a light positioning of the insole in close proximity of the foot wound using at least one of a loosely wrapped bandage and a gauze.

In yet another aspect, the process would exclude any use of petroleum jelly on the gauze.

In yet another aspect, the method can apply the wound treatment composition to an insole inserted into a weight offloading boot.

In yet another aspect, the method can apply the wound treatment composition to an insole wherein the insole would be utilized in conjunction with an Unna boot.

In yet another aspect, the gauze compression would be applied away from the foot ulcer. Once the ulcer dries, the boot can be removed and a sock can be used to separate the insole from the dry ulcer.

The present invention also describes a method of fabricating a wound treatment carrier, the method comprising steps of:
incorporating elemental Iodine into polyurethane;
dissolving the polyurethane and elemental Iodine combination into polyols forming a liquid composition;
treating the liquid composition with Di-isocyanates creating a treated composition; and
forming the treated composition into an insole.

The present invention also describes a method of fabricating a wound treatment carrier, the method comprising steps of:
forming a base layer;
applying a wound treatment composition to the base layer.
assembling a top layer to the base layer creating the insole.

The present invention also describes a method of fabricating a wound treatment carrier, the method comprising steps of:
forming a base layer;
forming a central layer;
forming a top layer;
applying a wound treatment composition to at least one of the base layer and the central layer.
assembling the central layer between the top layer and the base layer creating the insole.

The present invention also describes a method of fabricating a wound treatment carrier, the method comprising steps of:
forming a base layer;
forming at least one central layer;
forming a top layer;
applying a wound treatment composition to at least one of the base layer and one or more of the at least one central layer.
assembling each of the at least one central layer between the top layer and the base layer creating the insole.

The present invention also describes a method of fabricating a wound treatment carrier, the method comprising steps of:
forming a base layer;
forming at least one central layer;
forming a top layer;
applying a wound treatment composition to at least one of the base layer and each of the at least one central layer.
assembling each of the at least one central layer between the top layer and the base layer creating the insole.

The present invention also describes a method of fabricating a wound treatment carrier, the method comprising steps of:
forming a base layer of an insole;
applying a microporous polyurethane foam impregnated with a wound treatment composition to a top surface of the base layer.

In yet another aspect, the base layer of the insole is formed to contour to a bottom surface of a patient's foot.

In yet another aspect, the base layer of the insole is formed to have a contour similar to a contour of the bottom surface of the patient's foot.

In yet another aspect, the base layer of the insole is formed to have a contour generally resembling the contour of the bottom surface of the patient's foot.

In yet another aspect, the insole can be impregnated with iodine and used to treat foot odor.

In yet another aspect, the insole can be impregnated with iodine and used to treat foot odor caused by athlete's foot.

In yet another aspect, a wound treatment composition can be applied to a carrier material by soaking the carrier material in a volume of wound treatment material.

In yet another aspect, a wound treatment composition can be applied to a carrier material by soaking the carrier material in a volume of wound treatment material, then letting the carrier material dry.

In yet another aspect, a wound treatment composition can be applied to a carrier material using an applicator.

In yet another aspect, a wound treatment composition can be applied to the carrier material using a spray applicator.

In yet another aspect, a wound treatment composition can be applied to the carrier material using the spray applicator, where the spray applicator includes a container and a spray dispenser.

In yet another aspect, a wound treatment composition can be applied to the carrier material using the spray applicator, where the spray applicator includes a container and a pump spray dispenser.

In yet another aspect, a wound treatment composition can be applied to the carrier material using the spray applicator, where the spray applicator includes a container and a childproof dispenser.

In another aspect, the malodorous odor and an associated source of the malodorous odor treatment barrier body further comprising an aperture.

In another aspect, the malodorous odor and an associated source of the malodorous odor treatment barrier body further comprising an aperture, wherein the aperture is sized to receive the malodorous odor and an associated source of the malodorous odor treatment composition carrier.

In another aspect, the malodorous odor and an associated source of the malodorous odor treatment composition carrier is inserted through the aperture of the malodorous odor and an associated source of the malodorous odor treatment barrier body.

In another aspect, the malodorous odor and an associated source of the malodorous odor treatment composition carrier is threadably assembled into the aperture of the malodorous odor and an associated source of the malodorous odor treatment barrier body.

In another aspect, the malodorous odor and an associated source of the malodorous odor treatment composition carrier is assembled into a receiver inserted into the aperture of the malodorous odor and an associated source of the malodorous odor treatment barrier body.

In another aspect, the malodorous odor and an associated source of the malodorous odor treatment composition carrier is threadably assembled into a threaded receiver inserted into the aperture of the malodorous odor and an associated source of the malodorous odor treatment barrier body.

In another aspect, the malodorous odor and an associated source of the malodorous odor treatment barrier body further comprising a malodorous odor and an associated source of the malodorous odor viewing aperture.

In another aspect, the malodorous odor and an associated source of the malodorous odor treatment barrier body further comprising a malodorous odor and an associated source of the malodorous odor viewing aperture, the malodorous odor and an associated source of the malodorous odor viewing aperture being formed through the material of the malodorous odor and an associated source of the malodorous odor treatment barrier body.

In another aspect, the malodorous odor and an associated source of the malodorous odor treatment barrier body further comprising a malodorous odor and an associated source of the malodorous odor viewing aperture, the malodorous odor and an associated source of the malodorous odor viewing aperture being formed through the material of a domed portion of the malodorous odor and an associated source of the malodorous odor treatment barrier body.

In another aspect, the malodorous odor and an associated source of the malodorous odor treatment barrier body further comprising a malodorous odor and an associated source of the malodorous odor viewing aperture, the malodorous odor and an associated source of the malodorous odor viewing aperture being sealed by a clear material.

In another aspect, the contactless malodorous odor and an associated source of the malodorous odor treatment barrier assembly further comprising heat generating element.

In another aspect, the heat generating element being located within the malodorous odor and an associated source of the malodorous odor treatment barrier body.

In another aspect, the malodorous odor and an associated source of the malodorous odor treatment composition carrier further comprising a mechanism to adjust a rate of dispensing of dispensed medication.

In another aspect, the malodorous odor and an associated source of the malodorous odor treatment composition carrier further comprising a mechanism to adjust a rate of dispensing of dispensed medication, wherein the mechanism is programmable.

In another aspect, the malodorous odor and an associated source of the malodorous odor treatment composition carrier further comprising a mechanism to adjust a rate of dispensing of dispensed medication, wherein the mechanism is programmable enabling programmable dispending of the medication.

In another aspect, the malodorous odor and an associated source of the malodorous odor treatment composition carrier further comprising a mechanism to adjust a rate of dispensing of dispensed medication, wherein the mechanism is programmable enabling programmable dispending of the medication, wherein the programming is provided via a software instruction set.

In another aspect, the malodorous odor and an associated source of the malodorous odor treatment composition carrier further comprising a mechanism to adjust a rate of dispensing of dispensed medication, wherein the mechanism is programmable enabling programmable dispending of the medication, wherein the programming is provided via a software instruction set, wherein the instruction set includes a time and volume for dispensing the medication.

In another aspect, the malodorous odor and an associated source of the malodorous odor treatment composition carrier further comprising a mechanism to adjust a rate of dispensing of dispensed medication, wherein the mechanism is programmable enabling programmable dispending of the medication, wherein the programming is provided via a software instruction set, wherein the instruction set includes time periods and a volume for dispensing the medication associated with each time period.

In another aspect, the malodorous odor and an associated source of the malodorous odor treatment composition carrier further comprising a mechanism to adjust a rate of dispensing of dispensed medication, wherein the mechanism is programmable enabling programmable dispending of the medication, wherein the programming is provided via a software instruction set provided by a medical professional.

In another aspect, the malodorous odor and an associated source of the malodorous odor treatment composition carrier further comprising a mechanism to adjust a rate of dispensing of dispensed medication, wherein the mechanism is programmable enabling programmable dispending of the medication, wherein the programming is provided via a software instruction set provided by a medical professional via an internet.

In another aspect, the malodorous odor and an associated source of the malodorous odor treatment composition carrier further comprising a mechanism to adjust a rate of dispensing of dispensed medication, wherein the rate adjusting mechanism rotates a member to adjust a dispensing opening in the malodorous odor and an associated source of the malodorous odor treatment composition carrier.

In another aspect, the malodorous odor and an associated source of the malodorous odor treatment barrier body further comprising a mechanism to nebulizer the dispensed medication.

In another aspect, the malodorous odor and an associated source of the malodorous odor treatment barrier body further comprising a mechanism to circulate the dispensed medication.

In another aspect, the malodorous odor and an associated source of the malodorous odor treatment barrier body further comprising a mechanism to circulate the dispensed medication, wherein the circulation mechanism is a fan.

The present invention also describes a method of treating a malodorous odor and an associated source of the malodorous odor, wherein the method of treatment comprising steps of:
inserting an odor treatment composition carrier and a garment into an enclosure, the odor treatment composition carrier carrying an odor treatment composition in at least one of a solid state and a liquid state, the garment being designed to be worn in an area adjacent to the associated source of the malodorous odor, wherein the enclosure is airtight when sealed,
sealing the enclosure creating an airtight enclosed volume;
emitting the odor treatment composition in a gaseous state from the odor treatment composition carrier within the enclosed volume of the enclosure;
impregnating the garment with the odor treatment composition;
placing the garment onto the patient proximate the source of the malodorous odor; and
wearing the object while the odor treatment composition treats the source of the malodorous odor.

In another aspect, the odor treatment composition includes an iodine.

In yet another aspect, the odor treatment composition is an iodine.

In yet another aspect, the method of treating a malodorous odor and an associated source of the malodorous odor further comprising a step of removing the garment from the patient.

In yet another aspect, the method of treating a malodorous odor and an associated source of the malodorous odor further comprising a step of securing the wound treatment enclosure to the skin of a patient at a location proximate the wound in a manner where the wound treatment enclosure covers the wound.

In yet another aspect, the method of treating a malodorous odor and an associated source of the malodorous odor further comprising a step of securing the wound treatment enclosure to the skin of a patient at a location proximate the wound in a manner where the wound treatment enclosure completely covers the wound.

In yet another aspect, the method of treating a malodorous odor and an associated source of the malodorous odor further comprising a step of securing the wound treatment enclosure to the skin of a patient at a location proximate the wound in a manner where the wound treatment enclosure completely encases the wound.

In yet another aspect, the method of treating a malodorous odor and an associated source of the malodorous odor further comprising a step of securing the wound treatment enclosure to the skin of a patient is accomplished using an adhesive provided on a contacting surface of the wound treatment enclosure.

In yet another aspect, the method of treating a malodorous odor and an associated source of the malodorous odor further comprising a step of securing the wound treatment enclosure to the skin of a patient is accomplished using an adhesive provided on a contacting surface of a flange circumscribing the wound treatment enclosure.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient, wherein the absorbent material follows a circumference of the body of the wound treatment enclosure.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient, wherein the absorbent material is bonded to the wound treatment enclosure.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient, wherein the absorbent material is bonded to a flange circumscribing the wound treatment enclosure.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient, wherein the absorbent material provides a seal between facing surfaces of the wound treatment enclosure and the skin of the patient.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient, wherein the absorbent material provides a complete seal between facing surfaces of the wound treatment enclosure and the skin of the patient.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient, wherein a first side of the absorbent material is bonded to the wound treatment enclosure and a second, opposite side is bonded to the skin of the patient.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient, wherein a first side of the absorbent material is bonded to the wound treatment enclosure and a second, opposite side is bonded to the skin of the patient, wherein at least one bonded interface employs a double sided adhesive.

In yet another aspect, the method of treating a malodorous odor and an associated source of the malodorous odor further comprising a step of placing the wound treatment enclosure against the skin of a patient includes placement of an absorbent material between the wound treatment enclosure and the skin of the patient, wherein a first side of the absorbent material is bonded to the wound treatment enclosure and a second, opposite side is bonded to the skin of the patient, wherein both bonded interfaces employ the double sided adhesive.

In yet another aspect, the method of treating a malodorous odor and an associated source of the malodorous odor further comprising a step of inserting the odor treatment composition carrier through an aperture formed through a body of the wound treatment enclosure.

In yet another aspect, the method of treating a malodorous odor and an associated source of the malodorous odor further comprising a step of inserting the odor treatment composition carrier through an aperture formed through a body of the wound treatment enclosure, wherein the odor treatment composition carrier is threadably secured within the aperture.

In yet another aspect, the method of treating a malodorous odor and an associated source of the malodorous odor further comprising a step of inserting the odor treatment composition carrier through an aperture formed through a body of the wound treatment enclosure, wherein the odor treatment composition carrier is threadably secured to a threaded receiver inserted within the aperture formed through a body of the wound treatment enclosure.

In yet another aspect, the method of treating a malodorous odor and an associated source of the malodorous odor further comprising a step of inspecting the wound by viewing the wound through a viewing port formed through the body of the wound treatment enclosure.

In yet another aspect, the method of treating a malodorous odor and an associated source of the malodorous odor further comprising a step of inspecting the wound by viewing the wound through a clear material provided with the viewing port formed through the body of the wound treatment enclosure.

In yet another aspect, the odor treatment composition carrier is provided in a form of an insole.

In yet another aspect, the garment is provided in a form of a footwear and the odor treatment composition carrier is provided in a form of an insole, the method further comprising a step of inserting the insole into the footwear.

In yet another aspect, the method further comprising a step of orienting the garment inside out prior to sealing the enclosure.

In yet another aspect, the garment is provided in a form of at least one of a shirt, pants, shorts, socks, underwear, a bandage, and the like.

In another aspect, the garment is provided in a form of a footwear and the odor treatment composition carrier is provided in a form of an insole, the method further comprising steps of:
inserting the insole into the footwear; and
removing the insole from the footwear prior to placing the footwear onto a foot of the patient.

In another aspect, the garment is provided in a form of a footwear, the method further comprising a step of inserting the odor treatment composition carrier into the footwear.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a partially cut-away isometric bottom, front view of an exemplary contactless wound treatment barrier assembly in accordance with the present invention, the exemplary contactless wound treatment barrier assembly shown being placed over an exemplary wound;

FIG. 2 presents an isometric exploded assembly bottom, front view of the exemplary contactless wound treatment barrier assembly originally introduced in FIG. 1;

FIG. 3 presents a sectioned front view of the exemplary contactless wound treatment barrier assembly originally introduced in FIG. 1, the exemplary contactless wound treatment barrier assembly shown secured in place over an exemplary wound;

FIG. 4 presents an isometric bottom, front view of a variant of the exemplary contactless wound treatment barrier assembly originally introduced in FIG. 1;

FIG. 5 presents sectioned front view of a first enhanced version of the exemplary contactless wound treatment barrier assembly originally introduced in FIG. 1;

FIG. 6 presents sectioned front view of a second enhanced version of the exemplary contactless wound treatment barrier assembly originally introduced in FIG. 1;

FIG. 7 presents an isometric exploded assembly side view of an alternative exemplary contactless wound treatment barrier assembly applying the same concept and method of use, wherein the alternative exemplary contactless wound treatment barrier assembly is provided as a contactless wound treatment shoe insole for a shoe for treatment of a wound located on a patient's foot;

FIG. 8 presents an isometric exploded assembly side view of an alternative wound treatment shoe insole for a shoe;

FIG. 9 presents an isometric exploded assembly side view of a modified version of the wound treatment shoe insole for a shoe originally introduced in FIG. 8;

FIG. 10 presents a variety of exemplary chemical arrangements that are all suitable for use as a wound treatment composition with the contactless wound treatment barrier assembly;

FIG. 11 presents an exemplary contactless wound treatment flow diagram describing a method of using the exemplary contactless wound treatment barrier assembly for applying a wound treatment composition in gaseous form to a wound;

FIG. 12 presents an exemplary flow diagram describing a method of fabricating an insole of a polyurethane, the polyurethane comprising elemental iodine and Di-isocyanates;

FIG. 13 presents an isometric view of an exemplary insole for a shoe, the insole being fabricated including a laminated microporous polyurethane foam (high density polyurethane open cell material with memory elasticity), wherein iodine is impregnated into the microporous polyurethane foam material of the insole;

FIG. 14 presents an isometric view of a spray bottle applying or reapplying a wound treatment composition onto a sole of a shoe;

FIG. 15 presents a perspective bottom view of an exemplary wound on a patient's foot, the illustration presenting the wound following one (1) week of treatment;

FIG. 16 presents a perspective bottom view of the exemplary wound on a patient's foot originally introduced in FIG. 15, the illustration presenting the wound following two (2) weeks of treatment;

FIG. 17 presents a perspective bottom view of the exemplary wound on a patient's foot originally introduced in FIG. 15, the illustration presenting the wound following three (3) weeks of treatment;

FIG. 18 presents an exemplary flow diagram describing a method of treating a malodorous odor and a source of the malodorous odor;

FIG. 19 presents an isometric top exploded assembly view of an exemplary serviceable wound treatment apparatus;

FIG. 20 presents an isometric bottom section view of the exemplary serviceable wound treatment apparatus originally introduced in FIG. 19, the section being taken along section line 20-20 of FIG. 19;

FIG. 20 presents an isometric bottom exploded assembly section view of the exemplary serviceable wound treatment apparatus originally introduced in FIG. 19, the section being taken along section line 20-20 of FIG. 19, the illustration presenting the adhesive and adhesive cover as an exploded assembly;

FIG. 21 presents an isometric bottom assembly section view of the exemplary serviceable wound treatment apparatus originally introduced in FIG. 19, the section being taken along section line 20-20 of FIG. 19, the illustration presenting the adhesive and adhesive cover assembled to a flange of a wound treatment apparatus body;

FIG. 22 presents an isometric bottom exploded assembly section view of the exemplary serviceable wound treatment apparatus originally introduced in FIG. 19 being placed upon a skin of a patient for treatment of a wound, the section being taken along section line 20-20 of FIG. 19, the wound treatment composition carrier being inserted into the exemplary serviceable wound treatment apparatus;

FIG. 23 presents an isometric bottom assembly section view of the exemplary serviceable wound treatment apparatus originally introduced in FIG. 19 secured to the skin of the patient for treatment of the wound, the section being taken along section line 20-20 of FIG. 19, the wound treatment composition carrier as inserted into the exemplary serviceable wound treatment apparatus;

FIG. 24 presents an isometric bottom exploded assembly section view of a variant of the exemplary serviceable wound treatment apparatus originally introduced in FIG. 19, the variant introduces a threadable component for receiving and retaining the wound treatment composition carrier, the illustration being similar to FIG. 22;

FIG. 25 presents an isometric bottom assembly section view of the variant of the exemplary serviceable wound treatment apparatus originally introduced in FIG. 19, the wound treatment composition carrier, the illustration being similar to FIG. 22;

FIG. 26 presents an isometric bottom exploded assembly section view of the exemplary serviceable wound treatment apparatus originally introduced in FIG. 19, the section being taken along section line 20-20 of FIG. 19, the illustration introducing an absorbent material for placement between the serviceable wound treatment apparatus and the skin of the patient;

FIG. 27 presents an isometric bottom assembly section view of the exemplary serviceable wound treatment apparatus originally introduced in FIG. 19, the section being taken along section line 20-20 of FIG. 19, the illustration presenting the absorbent material bonded to the respective surface of the serviceable wound treatment apparatus;

FIG. 28 presents a sectioned side elevation view of an alternative exemplary serviceable wound treatment apparatus, wherein the alternative exemplary serviceable wound treatment apparatus is provided in a shape of a foot covering.

Like reference numerals refer to like parts throughout the several views of the drawings.

### DETAILED DESCRIPTION OF THE PRESENT INVENTION

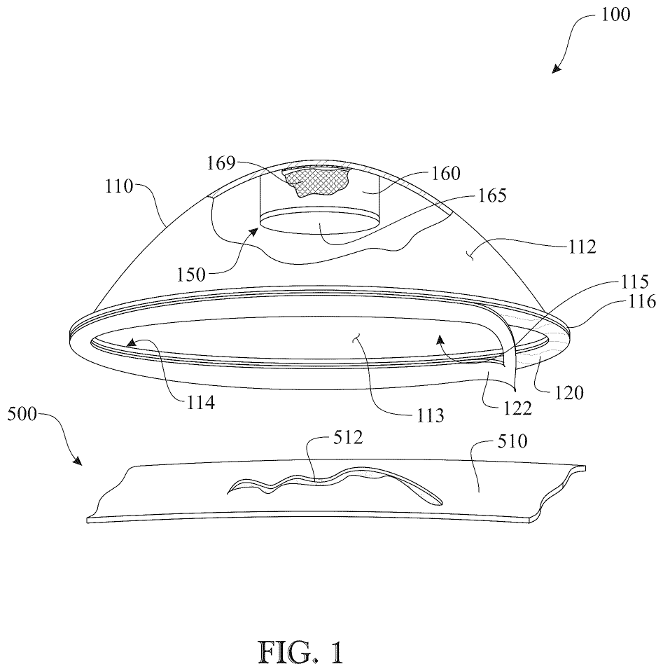

Detailed embodiments of the present invention are disclosed herein. It will be understood that the disclosed embodiments are merely exemplary of the invention that may be embodied in various and alternative forms. The figures are not necessarily to scale, and some features may be exaggerated or minimized to show details of particular embodiments, features, or elements. Specific structural and functional details, dimensions, or shapes disclosed herein are not limiting but serve as a basis for the claims and for teaching a person of ordinary skill in the art the described and claimed features of embodiments of the present invention. The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The present invention applies the benefits of antimicrobial and healing properties found in certain proven agents used on wound care treatment, but whose wound contact delivery becomes contraindicated due to toxicity or the inability to keep the wound from getting irritated, and therefore slowing or regressing the healing process. The present invention overcomes these problems by providing an apparatus enabling delivery of the active agents to the wound in a gaseous state, in a contactless fashion, while maintaining a constant delivery of the agent in gaseous form, and keeping the atmosphere around/above the wound in a humidity controlled and oxygen controlled environment. The wound treatment application apparatus can be designed to allow air/oxygen in contact with the wound (aerobic and breathable during the proliferative stage of healing), or designed to prevent air from getting in contact with the wound (hypoxic conditions during the inflammatory stage of healing).

The wound treatment application apparatus can be provided in a variety of form factors. The general concept of the present invention is presented in a first exemplary form factor, referred to as a contactless wound treatment barrier assembly 100 and illustrated in FIGS. 1 through 3. The contactless wound treatment barrier assembly 100 comprises a wound treatment barrier body 110 having a wound treatment barrier body exterior surface 112 and a wound treatment barrier body interior surface 113. The wound treatment barrier body 110 terminates at a wound treatment barrier body peripheral contact edge 114. A wound treatment barrier body support flange 116 is formed extending radially outward from a wound treatment barrier body peripheral contact edge 114 of the wound treatment barrier body 110. The wound treatment barrier body 110 is shaped to create a wound treatment barrier body interior volume 115 on one side of the wound treatment barrier body peripheral contact edge 114. The wound treatment barrier body 110 is of a size and shape where a top or upper area of the wound treatment barrier body interior surface 113 is distant from the wound treatment barrier body peripheral contact edge 114 or the wound treatment barrier body support flange 116. A primed wound treatment composition storage and delivery container assembly 150 is secured to the upper area of the wound treatment barrier body interior surface 113. The primed wound treatment composition storage and delivery container assembly 150 is of a height that is less than a distance between the top or upper area of the wound treatment barrier body interior surface 113 and the wound treatment barrier body support flange 116, ensuring that a lower surface of the primed wound treatment composition storage and delivery container assembly 150 remains elevated above a contacting surface or exemplary skin 510 of an exemplary injured region 500.

The exemplary wound treatment barrier body 110 is illustrated in accordance with a preferred shape, where the wound treatment barrier body peripheral contact edge 114 is circular in shape and the wound treatment barrier body 110 is formed having a domed shape. It is understood that the wound treatment barrier body 110 can be formed having any suitable shape to create a wound treatment barrier body interior volume 115. Examples include a hemispherical shape, a hemi-ellipsoid shape, a hemi-spheroid shape, a hemi-oblate spheroid shape, a hemi-prolate spheroid shape, a cone shape, a frustum shape, a tetrahedron shape, a triangular pyramid shape, a rectangular pyramid shape, a square pyramid shape, a hexagonal pyramid shape, a cube shape, a hexagonal prism shape, and the like.

The primed wound treatment composition storage and delivery container assembly 150 can be provided in any suitable format. The illustration presents a first exemplary configuration of a suitable primed wound treatment composition storage and delivery container assembly 150. The exemplary primed wound treatment composition storage and delivery container assembly 150 includes a primed wound treatment composition storage and delivery container closure 165 sealing a primed wound treatment composition carrier 169 within an interior of a primed wound treatment composition storage and delivery container body 160. A peripheral edge of the primed wound treatment composition storage and delivery container body attachment surface 164 is contiguous about a like shaped and sized peripheral edge of the primed wound treatment composition storage and delivery container body 160. The primed wound treatment composition storage and delivery container body 160 is shaped to form a cavity for retaining the primed wound treatment composition carrier 169. The primed wound treatment composition storage and delivery container body 160 includes a primed wound treatment composition storage and delivery container body exterior surface 162 and a primed wound treatment composition storage and delivery container body interior surface 163. The primed wound treatment composition storage and delivery container body interior surface 163 of the primed wound treatment composition storage and delivery container body 160 provides a peripheral wall defining the cavity. The primed wound treatment composition storage and delivery container body attachment surface 164 provides an interior bottom wall for the cavity. The primed wound treatment composition carrier 169 comprises a volume of wound treatment composition 167 applied to a wound treatment composition carrier 168.

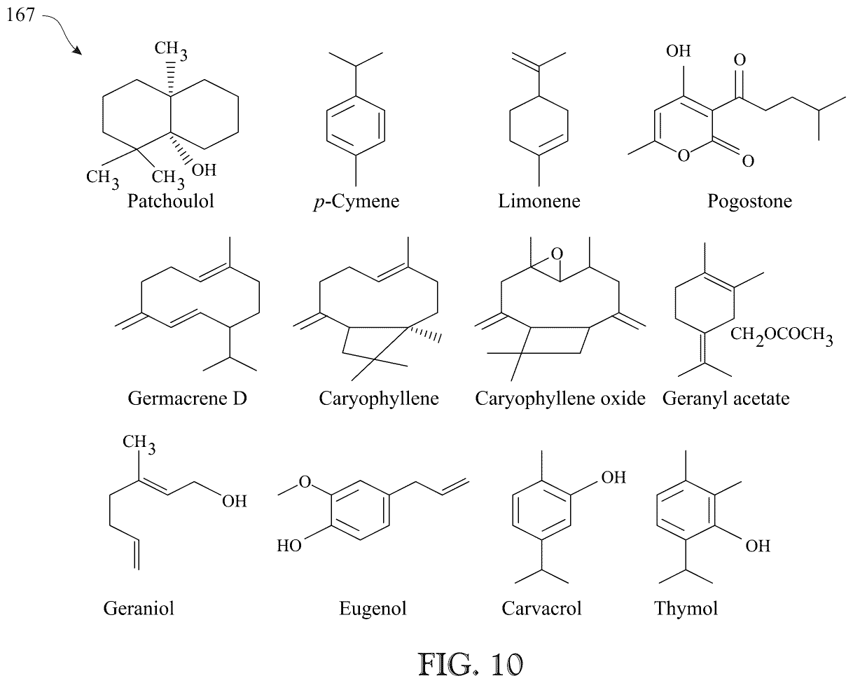

A listing of a number of optional wound treatment compositions 167 that can be applied to the wound treatment composition carrier 168 for treatment of a wound of a patient are listed in tables 1, 2 and 3 presented below. A diagrammatic representation of several exemplary wound treatment compositions 167 are illustrated and named accordingly in FIG. 10.

The following is a list of optional wound treatment compositions 167, including:
elemental Iodine,
iodoform,
ethanol,
isopropyl alcohol,
maltol,
3-(phenylamino) butan-2-one (schleiferon A),
3-(phenylimino) butan-2-one (schleiferon B),
acetoin and 2-phenylethylamine,
elemental sulfur,
nonanol,
decanol,
undecanol,
dodecanol,
NO gas releasing compounds such as S-Nitrosoglutathione,
1-hydroxy-2-oxo-3, 3-bis(2-aminoethyl) 1-triazene,
1-hydroxy-2-oxo-3, 3-bis(3-aminoethyl) 1-triazene,
1-hydroxy-2-oxo-3-(3-aminopropyl) 3-(4-aminobutyl) 1-triazene
Oxygen,
essential antimicrobial oils from oregano,
thyme,
clove,
lavender,
clary sage,
arborvitae,
active oils eucalyptol,
thymol,
menthol,
carvacrol, thymol,
cinnamic aldehyde,
eugenol,
p-cymene,
allicine,
calendula oil,
melaleuca oil,
lemongrass oil, and
the like.

The wound treatment composition carrier 168 can be fabricated having any suitable shape and size. The shape and size of the wound treatment composition carrier 168 would be such to snugly fit within the interior volume or cavity created by the primed wound treatment composition storage and delivery container body 160. For example, the wound treatment composition carrier 168 can be of a circular shape, where the peripheral edge is equal to or slightly greater than the like peripheral edge of the primed wound treatment composition storage and delivery container body interior surface 163. In another example, the wound treatment composition carrier 168 can be of a star shape, where a diameter defined by the distal points of the star shape is equal to or slightly greater than the like peripheral edge of the primed wound treatment composition storage and delivery container body interior surface 163. In another example, the wound treatment composition carrier 168 can be of a rectangular shape, where diagonal corners of the rectangular shape fits snugly within the primed wound treatment composition storage and delivery container body interior surface 163 of the primed wound treatment composition storage and delivery container body 160. The term snugly is defined as where a pliancy of the wound treatment composition carrier 168 compresses the wound treatment composition carrier 168 slightly when the wound treatment composition carrier 168 is inserted into the interior volume or cavity created by the primed wound treatment composition storage and delivery container body interior surface 163 of the primed wound treatment composition storage and delivery container body 160. Other shapes and sizes providing a snug fit can alternatively be considered, such as a hexagonal shape, an octagonal shape, a triangular shape, and the like. The thickness of the wound treatment composition carrier 168 can be equal to or less than a height of the primed wound treatment composition storage and delivery container body 160. The thickness of the wound treatment composition carrier 168 can be slightly larger than the height of the primed wound treatment composition storage and delivery container body 160 in a condition where the wound treatment composition carrier 168 is compressed when the primed wound treatment composition storage and delivery container closure 165 is secured to the primed wound treatment composition storage and delivery container body 160.

Although the primed wound treatment composition storage and delivery container body 160 is illustrated as having a cylindrical shape, it is also recognized that the primed wound treatment composition storage and delivery container body 160 can be of any suitable shape and size. The shape and size of the wound treatment composition carrier 168 would be configured accordingly to snugly fit therein.

In an alternative arrangement, the wound treatment composition carrier 168 is secured directly to the wound treatment barrier body interior surface 113. In this arrangement, the wound treatment composition carrier 168 would be preferably shaped and sized to rest against the wound treatment barrier body interior surface 113. The size and shape of a sidewall of the wound treatment composition carrier 168 can be shaped to optimize an exposed surface area of the wound treatment composition carrier 168. For example, a star shaped wound treatment composition carrier 168 would have a greater surface area than a like sized square or round shaped wound treatment composition carrier 168. The greater the exposed surface area, the greater the emission of the wound treatment composition 167 in a gaseous state.

The wound treatment composition carrier 168 can be fabricated of any suitable material having properties capable of absorbing a composition in a liquid state and emitting the composition in a gaseous state. Examples of suitable material can be a porous material, such as an open cell foam, an open cell polyurethane, a closed cell polyurethane, a reticulated polyurethane foam, silicone, rubber, silicone-rubber, a sponge; an absorbent material, such as a fabric, a carpet, and the like.

The wound treatment composition 167 can be any of the following:
Iodine, as tincture, or as iodine releasing compounds (Iodophores), other antimicrobial compounds, maltol, ethanol, isopropyl alcohol, isopropyl alcohol, maltol, 3-(phenylamino) butan-2-one (schleiferon A), 3-(phenylimino) butan-2-one (schleiferon B), acetoin and 2-phenylethylamine, elemental sulfur, nonanol, decanol, undecanol, dodecanol, NO gas releasing compounds such as the NONOates: S-Nitrosoglutathione, 1-hydroxy-2-oxo-3,3-bis(2-aminoethyl)1-triazene, 1-hydroxy-2-oxo-3,3-bis(3-aminoethyl)1-triazene, 1-hydroxy-2-oxo-3-(3-aminopropyl)3-(4-aminobutyl)1-triazene Oxygen, essential antimicrobial oils from oregano, thyme, clove, lavender, clary sage, melaleuca calendula and arborvitae, as well as the active oils eucalyptol, thymol, menthol, carvacrol, thymol, cinnamic aldehyde, eugenol, and p-cymene, allicine, calendula oil, melaleuca oil, lemongrass oil, and the like.

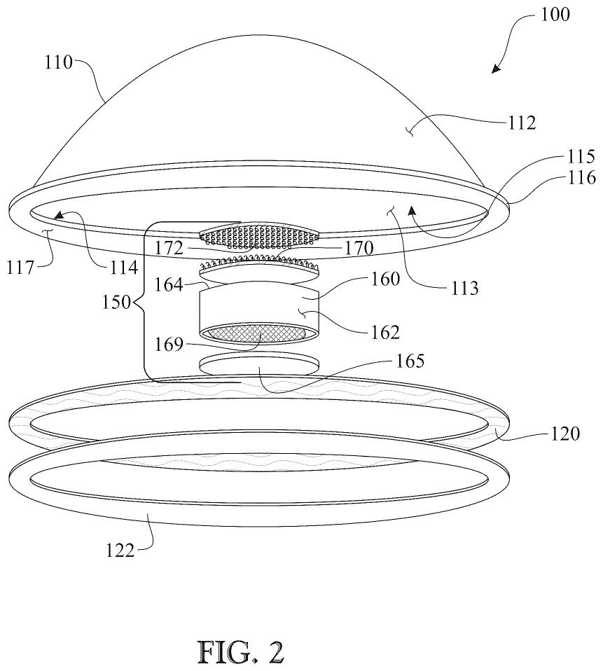

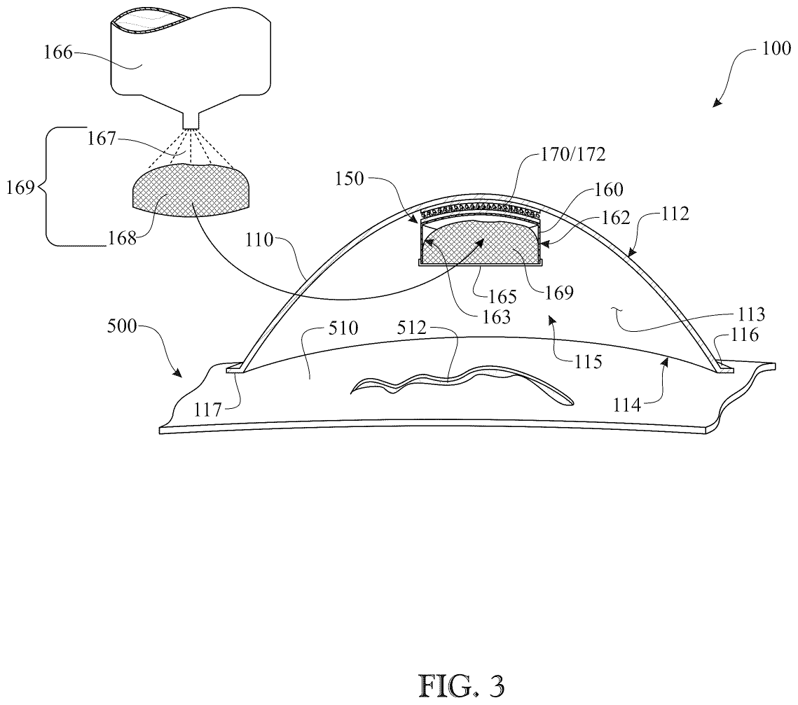

The wound treatment composition 167 can be applied to the wound treatment composition carrier 168 from any wound treatment composition container 166. Instructions or a prescription can be provided, where the instructions or prescription include a volume of wound treatment composition 167 that is to be applied to the wound treatment composition carrier 168. The volume can be based upon the cubic size of the wound treatment composition carrier 168, the treatment of the wound, and the like. In one application, the wound treatment composition container 166 can be a retail bottle, where the end user would dispense the wound treatment composition 167 from the wound treatment composition container 166 onto the wound treatment composition carrier 168. In a second application, the wound treatment composition container 166 can be a large delivery container, where the manufacturer would dispense the wound treatment composition 167 from the wound treatment composition container 166 onto the wound treatment composition carrier 168, then package the primed wound treatment composition carrier 169 within a sealed package for transport and delivery to any of a medical facility, a retailer, a distributor, an end user, and the like. The primed wound treatment composition storage and delivery container assembly 150 is secured to the top or upper area of the wound treatment barrier body interior surface 113 using any suitable attachment interface. This can include an adhesive or other bonding agent, a double sided tape adhesive, a dense hook and loop tape 170, 172 (as illustrated in FIGS. 2 and 3), mechanical fasteners (such as rivets, eyelets, nuts and bolts, a hook and eyelet, a snap stud and mating snap socket (receptacle), a staple or multiple staples, a tie (such as a string, a ribbon, and the like), or any other suitable fastener. In another alternative, the wound treatment barrier body 110 can include a pocket for receiving and retaining the primed wound treatment composition storage and delivery container assembly 150. The pocket would comprise an opening for dispensing of the primed wound treatment composition carrier 169 in aerated or gaseous form. Although the illustrations present the dense hook and loop tape 170, 172 as the exemplary attachment interface, it is recognized that the dense hook and loop tape 170, 172 can be replaced using any of the other suitable attachment interface, such as those described above or any other suitable attachment interface known by those skilled in the art.

In an enhanced packaging, the primed wound treatment composition carrier 169 can be filled with a healing gas, such nitrous-oxide (NO) or any NO releasing solid or liquid.

The contactless wound treatment barrier assembly 100 can be secured to the exemplary injured region 500 using any suitable attachment mechanism. The exemplary contactless wound treatment barrier assembly 100 presents one exemplary suitable attachment mechanism. The exemplary contactless wound treatment barrier assembly 100 includes a support flange adhesive 120 applied to a wound treatment barrier body support flange contact surface 117 of the wound treatment barrier body support flange 116, as best presented in the illustration shown in FIG. 2. The support flange adhesive 120 is protected by a support flange adhesive protective cover 122 until use.

The wound treatment barrier body 110 and wound treatment barrier body support flange 116 are preferably fabricated as a unitary component. The wound treatment barrier body 110 can be fabricated of any suitable material, including melt blown polypropylene, polyethylene, polyamide, polyurethane, polyester, polycarbonate, silicone. The thickness, shape, and material properties should be such to sufficiently retain a distance or non-contact arrangement between any surface of the contactless wound treatment barrier assembly 100 over the exemplary wound 512 (such as the primed wound treatment composition storage and delivery container assembly 150 and the wound treatment barrier body interior surface 113) and the exemplary wound 512. To create an anaerobic condition, the wound treatment barrier body exterior surface 112 of the wound treatment barrier body 110 can be sealed by applying a coating of flex seal silicone spray.

The contactless wound treatment barrier assembly 100 consists of a chamber-like or dome-like structure 110, that can be placed upon and secured to the exemplary skin 510 around the exemplary wound 512 using an adhesive 120 on the wound treatment barrier body support flange 116 extending radially outward from a contacting edge of the dome or chamber 110, to which a polymeric substrate (natural or man-made, such cellulose or polyurethane foam) carrying an antimicrobial and/or healing promoting solid or liquid, capable of subliming or evaporating, has been attached (or built in) to the interior surface 113 of the wound treatment barrier body 110 or the side of the wound treatment barrier body 110 facing the exemplary wound 512. The dome or chamber 110 can be air permeable to deliver the active agent under an aerobic condition or impermeable to deliver the active agent under an anaerobic condition. The ceiling and/or sidewalls of the device dome or chamber 110 is rigid enough and sufficiently distant from the exemplary wound 512 as not to come in contact with the exemplary wound 512, and to allow enough void space to prevent an accidental pressure over the wound treatment barrier body 110 to make any surface within an interior of the wound treatment barrier body 110 contact the exemplary wound 512. The rigidity can be determined by a thickness, a shape, material properties and the like that are considered when designing the wound treatment barrier body 110.

In an alternative arrangement, the primed wound treatment composition carrier 169 can be directly secured to the wound treatment barrier body interior surface 113 of the wound treatment barrier body 110 using any suitable assembly interface. In this configuration, since the primed wound treatment composition carrier 169 may be “wet”, a mechanical fastener is more desirable over an adhesive.

Several examples of the primed wound treatment composition carrier 169 are presented as follows:

### Example 1

A small piece of polyuthetane foam 168 of 1 square inch and 0.25 inch in thickness was soaked in a 5% solution of elemental iodine in alcohol (3 mL) 167. The alcohol is allowed to evaporate at room temperature on a flat piece of glass, in the open air. The dry, iodine infused, foam is attached to the inside of a standard size dust face mask using a staple or any other suitable mechanical fastener. Alternatively, the iodine impregnated polyurethane foam 169 is encased in a screw capped or lid covered container 160, and affixed to the wound treatment barrier body interior surface 113 of the wound treatment barrier body 110 with an adhesive (replacing the dense hook segment 170/dense loop segment 172. The screw cap or lid 165 is removed from the primed wound treatment composition storage and delivery container body 160 just prior to use.

### Example 2

A small piece of polyurethane foam 168 of 1 square inch and 0.25 inch in thickness is soaked in a 5% solution of elemental iodine in alcohol (3 mL) 167. The alcohol is not allowed to evaporate during the application and preparation process. The solution 167 is applied to the foam 168. The combination forming the primed wound treatment composition carrier 169 is attached directly to the wound treatment barrier body interior surface 113 of the wound treatment barrier body 110 using any suitable mechanical fastener, as described above.

### Example 3

A small piece of polyurethane foam 168 of 1 square inch and 0.25 inch in thickness is attached dry to the wound treatment barrier body interior surface 113 of the wound treatment barrier body 110, and then infused with the alcohol solution 167 in situ just before use.

### Example 4

Using a screw-cap cylindrical plastic container 160 having a volume of 4 mL; 50 mg of elemental iodine were placed therein and immediately covered with 3.5 mL of liquid silicone. After 24 hours at room temperature and vented to the air, the container 160 is sealed with the screw cap 165. As previously described, the primed wound treatment composition storage and delivery container assembly 150 containing the wound treatment composition 167 as described in this paragraph is attached to the wound treatment barrier body interior surface 113 of the wound treatment barrier body 110 using a bonding agent.

### Example 5

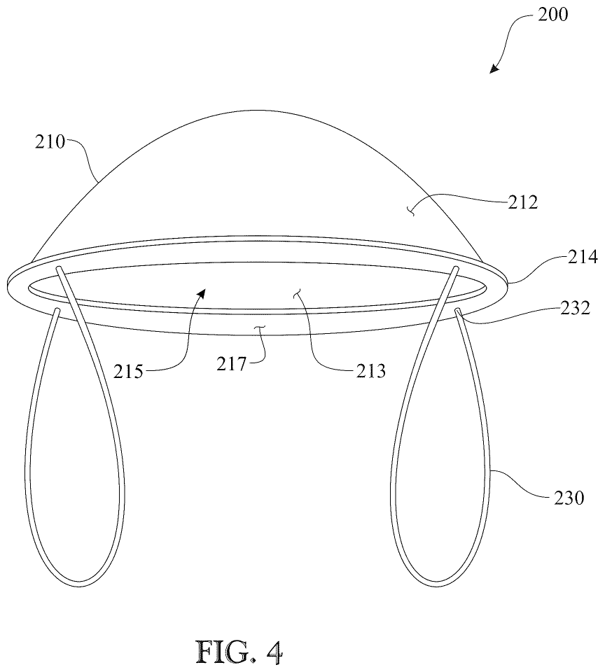

A one (1) week-old knee scrape wound (5 cm×2 cm) with no skin present, that have been treated with hydrogen peroxide for cleaning and topical triple antibiotic for healing, that had failed to start healing and was getting infected, was cleaned again with hydrogen peroxide to remove exudate and pus, and covered with the iodine gas chamber (vault) or wound treatment barrier body 110 (fabricated using a dust face mask) and retained in place using elastic bands 320 (FIG. 4) to keep the wound treatment barrier body 110 in place. After twelve hours (12h), the wound 512 had formed a multi-cracked scab and it appears to be healing. Additional two (2) days of wearing the wound treatment barrier body 110, showed a larger, drier, scab and no signs of pain or infection. The contactless wound treatment barrier assembly 100 delivers enough iodine to be noticed by a somewhat strong odor. The threshold to detect iodine in the air by smell is 0.9 parts per million (ppm). This concentration is manifold lower than the direct use of iodine tincture and comparable to PVP-I, 10% solution (2 to 22 ppm of free iodine). Larger or smaller doses can be controlled by adding more active ingredient to the wound treatment composition carrier 168 or by increasing/decreasing the surface area of the wound treatment composition carrier 168.

A small piece of polyurethane foam 168 of 1 square inch and 0.25 inch in thickness was attached dry to the wound treatment barrier body interior surface 113 of the wound treatment barrier body 110, and then infused with the alcohol solution 167 in situ just before use.

Another benefit of using the contactless wound treatment barrier assembly 100 is that the contactless wound treatment barrier assembly 100 can reduce or eliminate malodorous odors, such as those caused by bacterial infections, fungal infections, or those commonly associated with infections of wounds. In an application directed to reduce or eliminate malodorous odors the contactless wound treatment barrier assembly 100 can more appropriately be referred to as a malodorous odor treatment barrier assembly 100 and the components can be referred to by a like adjective.

The contactless wound treatment barrier assembly 100 is configured to be applied to the patient using the support flange adhesive 120. It is recognized that the support flange adhesive 120 may not be desirable for all applications. A contactless wound treatment barrier assembly 200, introduced in FIG. 4, illustrates an alternative attachment configuration. The contactless wound treatment barrier assembly 200 and the contactless wound treatment barrier assembly 100 include a number of like components. The contactless wound treatment barrier assembly 200 and the contactless wound treatment barrier assembly 100 would be nearly identical, with the only distinction being the attachment configuration for securing the contactless wound treatment barrier assembly 200 to the patient. Components of the contactless wound treatment barrier assembly 200 not shown in FIG. 4 which are included in the contactless wound treatment barrier assembly 100 are understood to be included in the contactless wound treatment barrier assembly 200 by reference. Like elements of the contactless wound treatment barrier assembly 200 and the contactless wound treatment barrier assembly 100 are numbered the same, wherein the elements of the contactless wound treatment barrier assembly 200 are preceded by the numeral “2”.

The contactless wound treatment barrier assembly 100 is configured to be secured to the patient at a location covering the exemplary wound 512 using the support flange adhesive 120. The alternative attachment configuration employs a contactless wound treatment barrier elastic retention member 230 attached to a wound treatment barrier body support flange 214 by an elastic retention member fastener 232. Both ends of the contactless wound treatment barrier elastic retention member 230 can be preassembled to the wound treatment barrier body support flange 214 by an elastic retention member fastener 232 or one end can be secured to the wound treatment barrier body support flange 214, while the second end of the contactless wound treatment barrier elastic retention member 230 can be secured to the wound treatment barrier body support flange 214 after wrapping the contactless wound treatment barrier elastic retention member 230 about the patient.

As with the contactless wound treatment barrier assembly 100, the contactless wound treatment barrier assembly 200 can reduce or eliminate malodorous odors, such as those caused by bacterial infections, fungal infections, or those commonly associated with infections of wounds. In an application directed to reduce or eliminate malodorous odors the contactless wound treatment barrier assembly 200 can more appropriately be referred to as a malodorous odor treatment barrier assembly 200 and the components can be referred to by a like adjective.

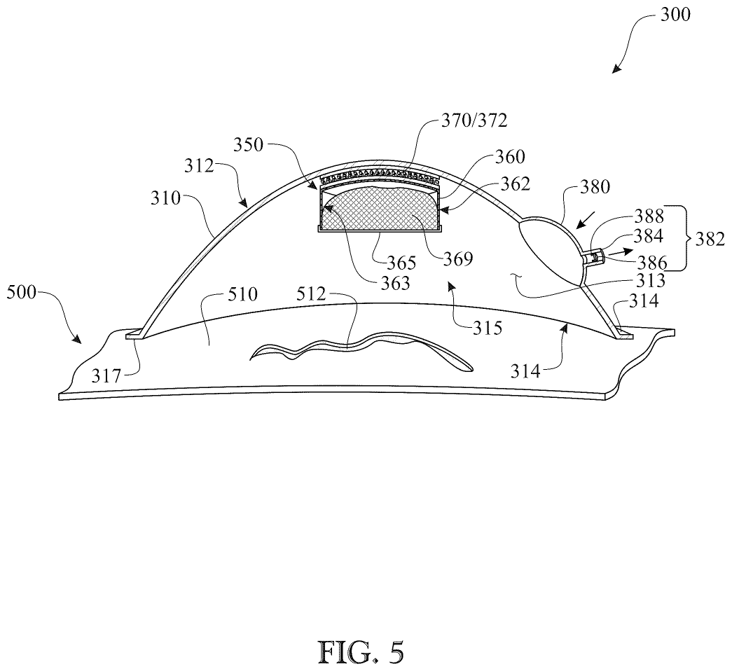

The contactless wound treatment barrier elastic retention member 230 is only a second exemplary configuration for securing the contactless wound treatment barrier assembly 200 to the patient. It is understood that any suitable attachment configuration can be utilized. This can include tape, a wrap, a wide bandage, or any other suitable attachment configuration known by those skilled in the art. Each of the securing devices would be routed circumscribing the body 110 (or the flange 116) around the limb associated with the wound. The contactless wound treatment barrier assembly 100 can be considered a basic or low tier version. A contactless wound treatment barrier assembly 300, introduced in FIG. 5, introduces a first enhanced variant of the contactless wound treatment barrier assembly 100. The contactless wound treatment barrier assembly 300 and the contactless wound treatment barrier assembly 100 include a number of like components. The contactless wound treatment barrier assembly 300 and the contactless wound treatment barrier assembly 100 would be nearly identical, with the distinction being the enhancement of a vacuum forming element. Like elements of the contactless wound treatment barrier assembly 300 and the contactless wound treatment barrier assembly 100 are numbered the same, wherein the elements of the contactless wound treatment barrier assembly 300 are preceded by the numeral “3”.

The contactless wound treatment barrier assembly 300 includes a vacuum creating element 380. The vacuum creating element 380 is a flexible element designed to displace a volume within an interior of a wound treatment barrier body 310. The displacement causes an increase in internal air pressure, causing entrapped air to escape from within an interior (defined by the wound treatment barrier body interior surface 313) of the wound treatment barrier body 310. In a basic configuration, the air can escape by slightly lifting the wound treatment barrier body support flange 314 from the exemplary skin 510, wherein the wound treatment barrier body interior surface 313 raises under the increased pressure.

In a more enhanced version, as illustrated in FIG. 5, the contactless wound treatment barrier assembly 300 includes a vacuum retaining check valve 382, where displacement causes entrapped air to vent through the vacuum retaining check valve 382. The vacuum retaining check valve 382 includes a vacuum retaining check valve flow control member 386 hingeably assembled within an interior of a vacuum retaining check valve tubular member 384. The vacuum retaining check valve flow control member 386 would seal against a vacuum retaining check valve flow control member seat 388 in a normal configuration and would be dislodged from the vacuum retaining check valve flow control member seat 388 when air pressure within the interior of the wound treatment barrier body 310 is greater than ambient air pressure external to the wound treatment barrier body 310. When the vacuum retaining check valve flow control member 386 is subjected to a condition where the air pressure within the interior of the wound treatment barrier body 310 is greater than ambient air pressure external to the wound treatment barrier body 310, the vacuum retaining check valve flow control member 386 would dislodge from the vacuum retaining check valve flow control member seat 388, enabling air to pass through the vacuum retaining check valve tubular member 384, this equalizing the air pressure within the interior of the wound treatment barrier body 310 and the ambient air pressure external to the wound treatment barrier body 310. The vacuum retaining check valve 382 can be integrated into the vacuum creating element 380 (as shown), or integrated into the wound treatment barrier body 310 at a separate location. The vacuum enables a better seal between the wound treatment barrier body support flange contact surface 317 and the exemplary skin 510. The vacuum also helps with the disbursement of the wound treatment composition 167 within the interior of the wound treatment barrier body 310.

As with the contactless wound treatment barrier assembly 100, the contactless wound treatment barrier assembly 300 can reduce or eliminate malodorous odors, such as those caused by bacterial infections, fungal infections, or those commonly associated with infections of wounds. In an application directed to reduce or eliminate malodorous odors the contactless wound treatment barrier assembly 300 can more appropriately be referred to as a malodorous odor treatment barrier assembly 300 and the components can be referred to by a like adjective.

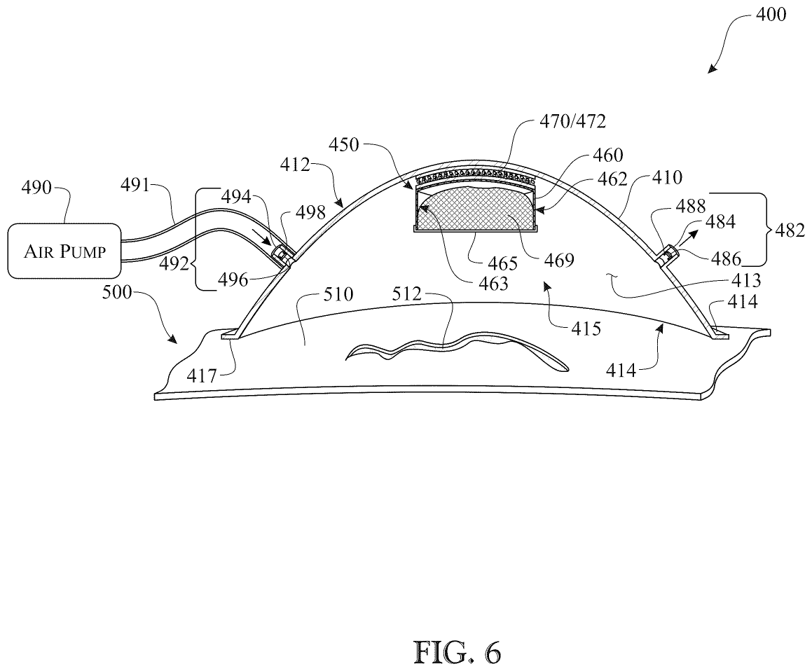

A contactless wound treatment barrier assembly 400, introduced in FIG. 6, introduces a second enhanced variant of the contactless wound treatment barrier assembly 100. The contactless wound treatment barrier assembly 400 and the contactless wound treatment barrier assembly 100 include a number of like components. The contactless wound treatment barrier assembly 400 and the contactless wound treatment barrier assembly 100 would be nearly identical, with the distinction being the enhancement of a gas injection system. Like elements of the contactless wound treatment barrier assembly 400 and the contactless wound treatment barrier assembly 100 are numbered the same, wherein the elements of the contactless wound treatment barrier assembly 400 are preceded by the numeral “4”.

The contactless wound treatment barrier assembly 400 includes an air pump 490, which delivers a gas to an interior volume 415 of a wound treatment barrier body 410. The air pump 490 is any suitable gas delivery system to deliver a gas to the interior volume 415 of the wound treatment barrier body 410. The gas can be oxygen, nitrogen, air, or any other desirable gas.

The gas or air is delivered to the interior volume 415 within the wound treatment barrier body 410 by pressure or flow generated from the air pump 490. The gas or air is transferred via an air pump delivery tube 491 to the interior volume 415 within the wound treatment barrier body 410. The air pump delivery tube 491 can be connected to an air fill check valve 492. The air fill check valve 492 includes an air fill check valve flow control member 496 hingeably assembled within an interior of an air fill check valve tubular member 494. The air fill check valve flow control member 496 seals against an air fill check valve flow control member seat 498 formed about a circumference of the interior of the air fill check valve tubular member 494. The air fill check valve 492 would operate in a manner opposite of the vacuum retaining check valve 382, where the pressure differential between the gas or air supplied from the air pump 490 causes the air fill check valve flow control member 496 to flex away from the air fill check valve flow control member seat 498, enabling passage of the gas or air through the air fill check valve 492, into the interior volume 415 of the wound treatment barrier body 410. When gas or air is not being delivered to the interior volume 415 of the wound treatment barrier body 410 from the air pump 490, the air fill check valve flow control member 496 remains seated against the air fill check valve flow control member seat 498, retaining entrapped gas or air within the interior volume 415 of the wound treatment barrier body 410.

The delivered gas or air displaces the existing gas or air within the interior volume 415 of the wound treatment barrier body 410. The displacement causes an increase in internal air pressure, causing entrapped gas or air to escape from within an interior (defined by the wound treatment barrier body interior surface 413) of the wound treatment barrier body 410. In a basic configuration, the air can escape by slightly lifting the wound treatment barrier body support flange 414 from the exemplary skin 510, wherein the wound treatment barrier body interior surface 413 raises under the increased pressure.

In a more enhanced version, as illustrated in FIG. 6, the contactless wound treatment barrier assembly 400 includes an interior pressure venting check valve 482, where displacement causes entrapped air to vent through the interior pressure venting check valve 482. The interior pressure venting check valve 482 includes an interior pressure venting check valve flow control member 486 hingeably assembled within an interior of an interior pressure venting check valve tubular member 484. The interior pressure venting check valve flow control member 486 would seal against an interior pressure venting check valve flow control member seat 488 in a normal configuration and would be dislodged from the interior pressure venting check valve flow control member seat 488 when air pressure within the interior of the wound treatment barrier body 410 is greater than ambient air pressure external to the wound treatment barrier body 410. When the interior pressure venting check valve flow control member 486 is subjected to a condition where the air pressure within the interior of the wound treatment barrier body 410 is greater than ambient air pressure external to the wound treatment barrier body 410, the interior pressure venting check valve flow control member 486 would dislodge from the interior pressure venting check valve flow control member seat 488, enabling air to pass through the interior pressure venting check valve tubular member 484, this equalizing the air pressure within the interior of the wound treatment barrier body 410 and the ambient air pressure external to the wound treatment barrier body 410. The vacuum enables a better seal between the wound treatment barrier body support flange contact surface 317 and the exemplary skin 510. The infusion of a gas or replacement of air within the interior of the wound treatment barrier body 410 aids in a process of healing the exemplary wound 512.

As with the contactless wound treatment barrier assembly 100, the contactless wound treatment barrier assembly 400 can reduce or eliminate malodorous odors, such as those caused by bacterial infections, fungal infections, or those commonly associated with infections of wounds. In an application directed to reduce or eliminate malodorous odors the contactless wound treatment barrier assembly 400 can more appropriately be referred to as a malodorous odor treatment barrier assembly 400 and the components can be referred to by a like adjective.

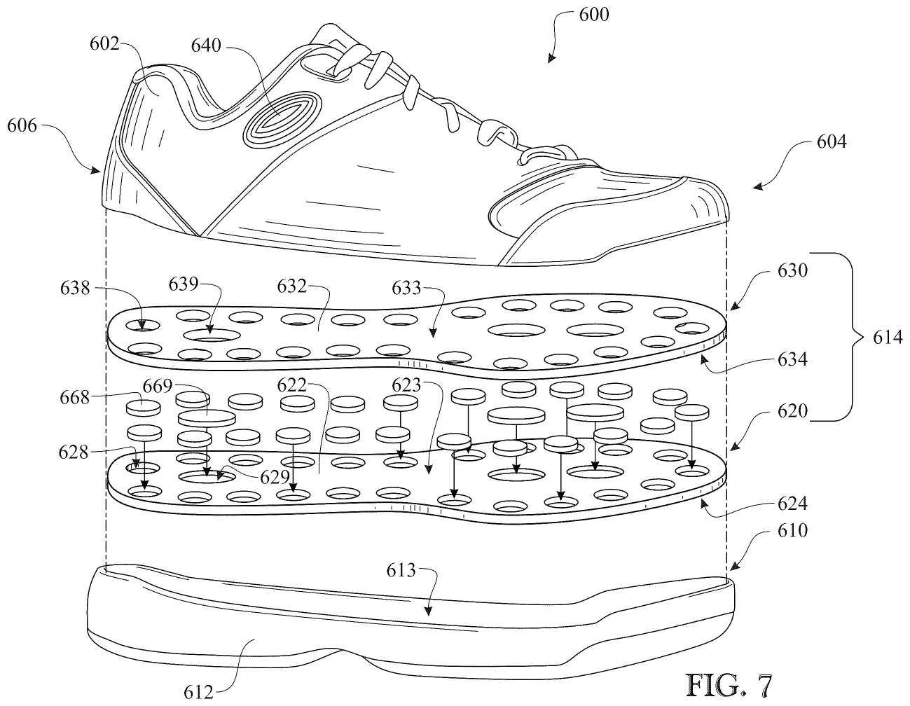

The general concept of the present invention is to apply a wound treatment composition 167 to a wound in a gaseous form exclusive of any contact to the wound. The same concept can be applied to other applicators. One example of an alternative applicator, a contactless wound treatment shoe assembly 600, is presented in FIG. 7. The contactless wound treatment shoe assembly 600 utilizes a contactless wound treatment shoe insole 614 comprising a laminated assembly including a contactless wound treatment shoe insole base 620 and a contactless wound treatment shoe insole upper 630.

The exemplary contactless wound treatment shoe assembly 600 includes elements common to a shoe, including a contactless wound treatment shoe upper 602, which is assembled to a contactless wound treatment shoe insole seat 613; the contactless wound treatment shoe insole seat 613 being defined partially by an interior surface of a contactless wound treatment shoe outsole 612 of a contactless wound treatment shoe sole 610. Orientation of the contactless wound treatment shoe assembly 600 can be referenced by a contactless wound treatment shoe toe section 604 and a contactless wound treatment shoe heel section 606.

The contactless wound treatment shoe insole base 620 is fabricated by shaping a material into a contactless wound treatment shoe insole base body 622. Orientation of the contactless wound treatment shoe insole base body 622 can be references by an insole base body upper surface 623, considered to be upward facing during use, and an insole base body lower surface 624, considered to be downward facing during use. Similarly, the contactless wound treatment shoe insole upper 630 is fabricated by shaping a material into a contactless wound treatment shoe insole upper body 632. Orientation of the contactless wound treatment shoe insole upper body 632 can be references by an insole base upper body upper surface 633, considered to be upward facing during use, and an insole base upper body lower surface 634, considered to downward facing during use.

The contactless wound treatment shoe insole base 620 and the contactless wound treatment shoe insole upper 630 can be fabricated of the same material, similar materials, or different materials.

The exemplary contactless wound treatment shoe insole 614 is fabricated by laminating the insole base body upper surface 623 of the contactless wound treatment shoe insole base body 622 and the insole base upper body lower surface 634 of the contactless wound treatment shoe insole upper body 632 to one another. It is recognized that other methods of manufacturing and arrangements can be employed to result in a contactless wound treatment shoe insole 614 having similar properties.

The contactless wound treatment shoe insole base 620 can include one or more contactless wound treatment shoe insole base receptacles 628, 629. The contactless wound treatment shoe insole base receptacles 628, 629 are located in various locations of the contactless wound treatment shoe insole base 620. The contactless wound treatment shoe insole base receptacles 628, 629 are preferably provided in locations known to have a higher incident rate of wounds on a person's foot.

The contactless wound treatment shoe insole base receptacles 628, 629 can be similar in size, different in size, or a mixture of various sizes, where some receptacles 628, 629 may be similar in size. The contactless wound treatment shoe insole base receptacles 628, 629 are formed having a size and shape to receive a respectively sized and shaped wound treatment composition insert 668, 669. Each wound treatment composition insert 668, 669 would be fabricated similar to the primed wound treatment composition carrier 169 described above. Each contactless wound treatment shoe insole base receptacle 628, 629 can pass through the 620 or include a bottom wall (not shown). The contactless wound treatment shoe insole base 620 can be fabricated of a flexible material enabling a snug or tight fit between each wound treatment composition insert 668, 669 and the receiving contactless wound treatment shoe insole base receptacle 628, 629. Alternatively, the base material (similar to the wound treatment composition carrier 168 of the primed wound treatment composition carrier 169) can be fabricated of a flexible material enabling a snug or tight fit between each wound treatment composition insert 668, 669 and the receiving contactless wound treatment shoe insole base receptacle 628, 629.

The exemplary contactless wound treatment shoe insole upper 630 includes a series of contactless wound treatment shoe insole upper treatment delivery apertures 638, 639 which are preferably located in vertical registration with the contactless wound treatment shoe insole base receptacles 628, 629 of the contactless wound treatment shoe insole base 620. The contactless wound treatment shoe insole upper 630 is fabricated of a material that would adequately support the patient's foot to maintain a distance between upper surfaces of the wound treatment composition inserts 668, 669 and the adjacent surface of the patient's foot, retaining a contactless treatment.

The contactless wound treatment shoe assembly 600 can additionally include an air flow generator 640. The air flow generator 640 can be configured in any of a variety of functions. In a first function, the air flow generator 640 can create an airflow within the interior of the contactless wound treatment shoe assembly 600, aiding in disbursing the wound treatment composition 167.

In a distribution process where the wound treatment composition inserts 668, 669 are prefilled with the wound treatment composition 167; the wound treatment composition inserts 668, 669 can be stored in a moisture impervious packaging until ready for use. Shortly prior to use, the wound treatment composition inserts 668, 669 would be removed from the moisture impervious packaging.

In use, the medical personnel or the patient would determine which of the contactless wound treatment shoe insole base receptacles 628, 629 are optimal for treatment of a respective wound. Upon determination of the optimal contactless wound treatment shoe insole base receptacles 628, 629, the respectively sized wound treatment composition inserts 668, 669 are located and inserted therein. This can be accomplished when the contactless wound treatment shoe insole 614 is the contactless wound treatment shoe assembly 600 removed from or inserted within the contactless wound treatment shoe assembly 600.

The patient would wear the contactless wound treatment shoe assemblies 600 for treatment, replacing the wound treatment composition inserts 668, 669 as needed, until the wounds are healed, or at a point of treatment where exposure to the wound treatment composition is no longer beneficial. To maintain a proper gait, it is suggested to include a pair of contactless wound treatment shoe insoles 614; one contactless wound treatment shoe insole 614 inserted into each shoe 600.

The series of contactless wound treatment shoe insole upper first treatment delivery apertures 638 and the series of contactless wound treatment shoe insole upper second treatment delivery aperture 639 of the contactless wound treatment shoe assembly 600 can create an airflow process, including a pressurization and a suction within each respective cavity. The pressurization and the suction would aid in drawing a gaseous state of the wound treatment composition 167 from the first wound treatment composition insert 668/second wound treatment composition insert 669 inserted in the respective cavity.

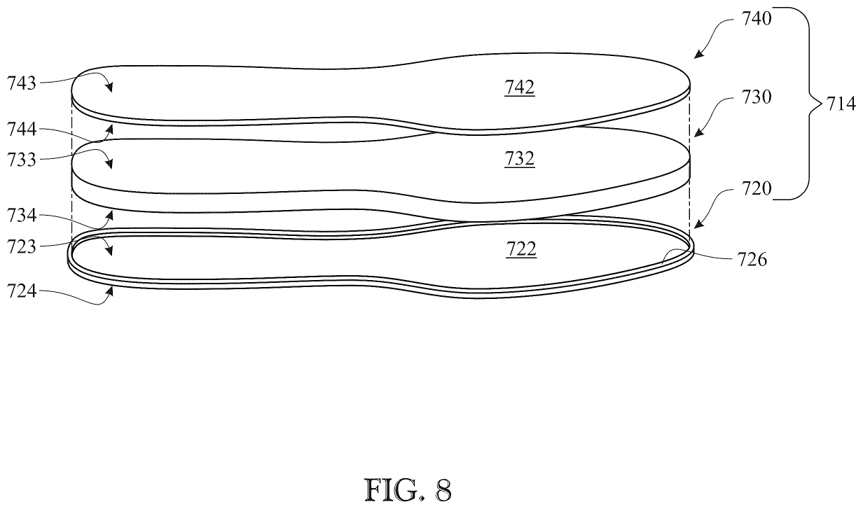

The contactless wound treatment shoe insole 614 is one exemplary configuration providing contactless wound treatment for a patient's foot. The concept can be provided in any of a number of variations. A contactless wound treatment shoe insole 714, illustrated in FIG. 8, presents a first variant of the contactless wound treatment shoe insole 614. The contactless wound treatment shoe insole 614 includes areas that are contactless to a wound. The contactless wound treatment shoe insole 714 is slightly deviated, where a porous surface exclusive of any wound treatment composition contacts the surface of the foot and the associated wound. The arrangement provides a dispersion system for the wound treatment composition, while maintaining adequate support and comfort to the patient.

In an additional application, the contactless wound treatment shoe insole 614 can be used to reduce or eliminate malodorous odors, such as those associated with athlete's foot, wounds, and other malodorous odors. In an application directed to reduce or eliminate malodorous odors the contactless wound treatment shoe insole 614 can more appropriately be referred to as a malodorous odor treatment shoe insole 614 and the components can be referred to by a like adjective.

The exemplary contactless wound treatment shoe insole 714 includes a contactless wound treatment shoe insole lower 720, a contactless wound treatment shoe insole core 730, and a contactless wound treatment shoe insole upper 740 laminated in sequence. The contactless wound treatment shoe insole lower 720 is fabricated of a moisture impervious material shaped into a contactless wound treatment shoe insole lower body 722, having an insole lower body top surface 723 and an insole lower body bottom surface 724. The contactless wound treatment shoe insole core 730 is fabricated of a porous material shaped into a contactless wound treatment shoe insole core body 732, having an insole core body top surface 733 and an insole core body bottom surface 734. The contactless wound treatment shoe insole core body 732 is fabricated of a material having properties enabling absorption of the wound treatment composition 167, while also enabling gaseous dispersion of the wound treatment composition 167. The contactless wound treatment shoe insole core body 732 would replicate the primed wound treatment composition carrier 169 described above. The contactless wound treatment shoe insole upper 740 is fabricated of a porous material shaped into a contactless wound treatment shoe insole upper body 742, having an insole upper body top surface 743 and an insole upper body bottom surface 744. The contactless wound treatment shoe insole upper body 742 is fabricated of a material and having a thickness and properties enabling support of the patient while maintaining a distance between the patient's foot and the insole core body top surface 733 of the contactless wound treatment shoe insole core 730.

To maintain overflow of a liquid state of the wound treatment composition 167, an insole lower body peripheral edge dam 726 can be formed around a periphery of an edge of the insole lower body top surface 723. The contactless wound treatment shoe insole core body 732 would be seated within the insole lower body peripheral edge dam 726. The insole core body bottom surface 734 and the insole lower body top surface 723 would be laminated to one another. Similarly, the insole upper body bottom surface 744 and the insole core body top surface 733 would be laminated to one another. When the contactless wound treatment shoe insole core body 732 is compressed, the contactless wound treatment shoe insole core body 732 may expulse a small volume of the wound treatment composition 167 in liquid form. Exclusive of the insole lower body peripheral edge dam 726, the expulsed volume of wound treatment composition 167 could puddle at the bottom of the shoe and wet the patient's foot. The insole lower body peripheral edge dam 726 contains any expulsed volume of wound treatment composition 167, thus avoiding any wetting of the patient's foot.

The contactless wound treatment shoe insole 714 can be stored in a moisture impervious packaging until ready for use. Shortly prior to use, the contactless wound treatment shoe insole 714 would be removed from the moisture impervious packaging.

When in use, the patient would preferably be wearing a sock or a loose bandage wrapped around of the treated foot. The at least one of a sock and a bandage aids in providing an additional barrier between the patient's foot and the wound treatment composition 167. During use, the wound treatment composition 167 would convert into a gaseous state and provide treatment of the wound. The gaseous state of the wound treatment composition 167 enables the wound treatment composition 167 to treat the wound without direct constant contact, as opposed to where a wound is normally subjected to direct contact with a liquid, a gel, or a solid composition. Additionally, to prevent future infections, the contactless wound treatment shoe insole 714 can made of any material that would allow the slow release of the wound treatment composition over weeks or even months. Examples of commercially available material that is suitable for this application include PORON®, POLYGEL®, or any other material with similar release properties.

The exemplary contactless wound treatment shoe insole 714 includes three layers 720, 730, 740 laminated together. It is understood that the contactless wound treatment shoe insole 714 can be fabricated of any number of layers, including a single insole body, two insole bodies, three insole bodies (as illustrated), four insole bodies, to nth number of insole bodies.

When the contactless wound treatment shoe insole 714 is fabricated having a single body (such as a 730), the wound treatment composition 167 can be applied to one or both surfaces, such as the exemplary insole body top surface 733, the exemplary insole body bottom surface 734, or both of the exemplary insole body top surface 733 and the exemplary insole body bottom surface 734. By applying the wound treatment composition 167 to the exemplary insole body bottom surface 734, it is less likely that the wound treatment composition 167 wet the patient's foot. Additionally, certain compositions that can be utilized as the wound treatment composition 167 have a tendency to temporarily stain the skin 510 of the patient. Therefore, by applying the wound treatment composition 167 to the exemplary insole body bottom surface 734, it reduces or eliminates a potential of staining the skin 510 of the patient.

The contactless wound treatment shoe insole 614 maintains a contactless relationship between a bottom of a patient's foot and the respective contacting surface (insole base upper body upper surface 633) of the contactless wound treatment shoe insole 614 by inclusion of the series of contactless wound treatment shoe insole upper first treatment delivery apertures 638 and contactless wound treatment shoe insole upper second treatment delivery apertures 639. The contactless wound treatment shoe insole 714 utilizes a modified concept, where the contactless wound treatment shoe insole core body 732 is impregnated with the wound treatment composition 167 or the wound treatment composition 167 is impregnated on a non-contacting surface. The contactless wound treatment shoe insole 714 provides contact between the bottom of a patient's foot and the respective contacting surface (insole upper body top surface 743) of the contactless wound treatment shoe insole 714, while maintaining a distance between the bottom of a patient's foot and the wound treatment composition 167. Therefore, the patient's foot is not subjected to any moisture or direct contact with the wound treatment composition 167.

As with the contactless wound treatment shoe insole 614, the contactless wound treatment shoe insole 714 can also be used to reduce or eliminate malodorous odors, such as those associated with athlete's foot, wounds, and other malodorous odors. In an application directed to reduce or eliminate malodorous odors the contactless wound treatment shoe insole 714 can more appropriately be referred to as a malodorous odor treatment shoe insole 714 and the components can be referred to by a like adjective.

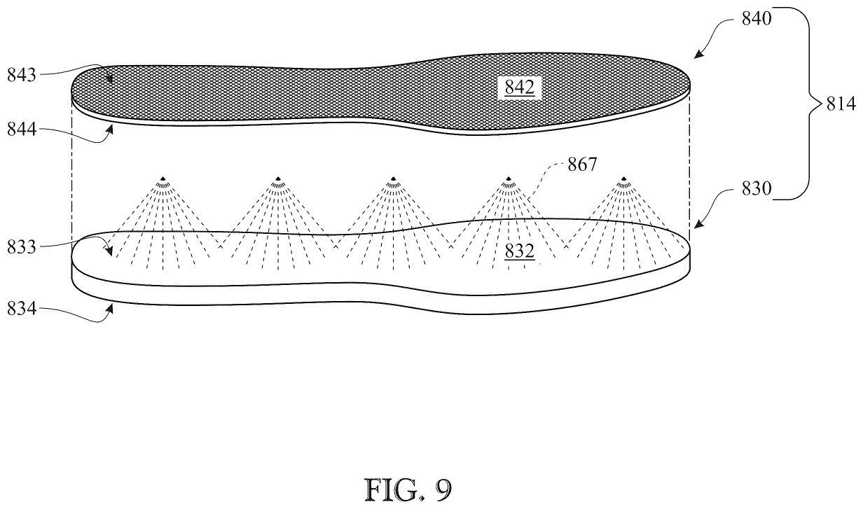

A wound treatment shoe insole 814 is introduced in FIG. 9. The wound treatment shoe insole 814 is a modified version of the contactless wound treatment shoe insole 714, where the wound treatment shoe insole 814 provides contact between the bottom of a patient's foot and the respective contacting surface (insole upper body top surface 843) of the wound treatment shoe insole wound treatment shoe insole 814, while maintaining a distance between the bottom of a patient's foot and the wound treatment composition 167. The wound treatment shoe insole 814 is fabricated having a wound treatment shoe insole upper 840 and a wound treatment shoe insole core 830 laminated to one another. The wound treatment shoe insole core 830 is fabricated of a wound treatment shoe insole core body 832 having an insole core body top surface 833 and an insole core body bottom surface 834. The wound treatment shoe insole core body 832 is preferably fabricated of any material that would allow the slow release of the wound treatment composition over weeks or even months. Examples of commercially available material that is suitable for this application include PORON®, POLYGEL®, or any other material with similar release properties. Similarly, the wound treatment shoe insole upper 840 is fabricated of a wound treatment shoe insole upper body 842 having an insole upper body top surface 843 and an insole upper body bottom surface 844. The insole upper body top surface 843 can be of a nylon material, a velvet material, a velour material, a polyester, Rayon, a satin material, or any other suitable material. Prior to lamination, a wound treatment composition 867 is applied to an insole core body top surface 833 of a wound treatment shoe insole core body 832 using any suitable application method. The wound treatment composition 867 is allowed to impregnate the wound treatment shoe insole core body 832 and the insole core body top surface 833 to dry. Once the wound treatment shoe insole core body 832 is properly prepared, the wound treatment shoe insole core body 832 and the wound treatment shoe insole upper body 842 are laminated to one another using any suitable laminating bonding medium and or bonding process. The wound treatment shoe insole core body 832 and the wound treatment shoe insole upper body 842 can be of any suitable size and shape during fabrication, such as provided in long continuous sheets of material. The wound treatment shoe insole 814 can be cut to shape following curing of the lamination process.

As with the contactless wound treatment shoe insole 614, the contactless wound treatment shoe insole 814 can also be used to reduce or eliminate malodorous odors, such as those associated with athlete's foot, wounds, and other malodorous odors. In an application directed to reduce or eliminate malodorous odors the contactless wound treatment shoe insole 814 can more appropriately be referred to as a malodorous odor treatment shoe insole 814 and the components can be referred to by a like adjective.

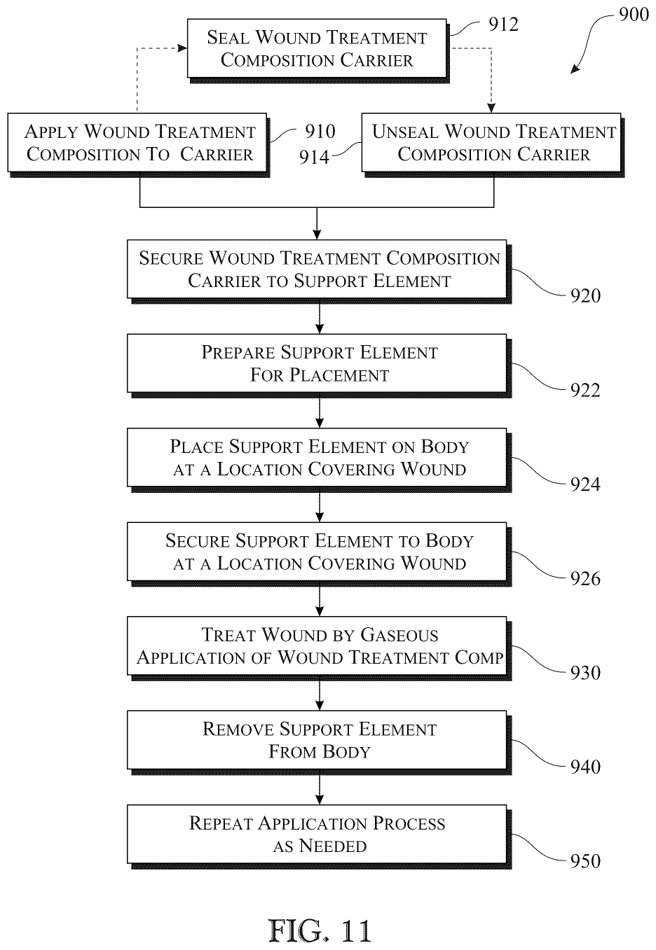

In general, each of the contactless wound treatment barrier assemblies 100, 200, 300, 400, and the contactless wound treatment shoe assembly 600 are configured to be used in a similar manner. The manner of use of described in a contactless wound treatment flow diagram 900 presented in FIG. 11. The contactless wound treatment flow diagram 900 initiates with a step of acquiring the associated components. The primed wound treatment composition carrier 169 is prepared by applying the wound treatment composition 167 to the wound treatment composition carrier 168 in accordance with an apply wound treatment composition to carrier step 910. This can be accomplished following one of two paths. Along a first path, the user would obtain a wound treatment composition carrier 168 in a dry state (prior to being considered as a primed wound treatment composition carrier 169). The user would obtain a volume of a wound treatment composition 167, wherein the wound treatment composition 167 would have a composition based upon the desired treatment. The user would complete the apply wound treatment composition to carrier step 910. The user would prepare the primed wound treatment composition carrier 169 by dispensing a volume of wound treatment composition 167 having the desired composition onto the wound treatment composition carrier 168 and allowing the wound treatment composition 167 to be absorbed into the material of the wound treatment composition carrier 168. Shortly after completion of the apply wound treatment composition to carrier step 910, the user would proceed to a secure wound composition carrier to interior of support element step 920; details of which are presented below.

Along an alternative path, the manufacturer would complete the apply wound treatment composition to carrier step 910. Shortly after completion of the apply wound treatment composition to carrier step 910, the manufacturer would place the primed wound treatment composition carrier 169 within a moisture impervious container in accordance with a seal wound treatment composition carrier step 912. The exemplary illustrations present one configuration for a moisture impervious container, more specifically, a primed wound treatment composition storage and delivery container body 160 sealed using a primed wound treatment composition storage and delivery container closure 165. In the exemplary arrangement, the primed wound treatment composition carrier 169 is sealed within the primed wound treatment composition storage and delivery container body 160 by the primed wound treatment composition storage and delivery container closure 165. The mating edge of the primed wound treatment composition storage and delivery container closure 165 can be sealed using a shrink wrap, a wax, or any other removable moisture impervious seal. The primed wound treatment composition carrier 169 can be sealed in any suitable moisture impervious container, such as the primed wound treatment composition storage and delivery container body 160 detailed above, a plastic bag, a metal container, a plastic container, a foil wrap, a plastic wrap, or any other suitable moisture impervious container. The material and the design of the moisture impervious container would retain the wound treatment composition 167 within the wound treatment composition carrier 168 until a time for use. Following this alternative path, prior to use, the primed wound treatment composition carrier 169 would be removed from the moisture impervious container in accordance with an unseal wound treatment composition carrier step 914 where the removal process would be determined by the packaging. In accordance with the exemplary illustrations, the primed wound treatment composition storage and delivery container closure 165 would be removed from the primed wound treatment composition storage and delivery container body 160, exposing the primed wound treatment composition carrier 169 to the atmosphere within the interior volume 115 of the wound treatment barrier body 110 in accordance with the unseal wound treatment composition carrier step 914.

The primed wound treatment composition carrier 169 is secured to a top region of the wound treatment barrier body interior surface 113 of the wound treatment barrier body 110. In the exemplary illustrations, the primed wound treatment composition carrier 169 is contained within the primed wound treatment composition storage and delivery container body 160. A dense hook segment 170 is secured to a primed wound treatment composition storage and delivery container body attachment surface 164 of the primed wound treatment composition storage and delivery container body 160. A dense loop segment 172 is secured to the wound treatment barrier body interior surface 113 of the wound treatment barrier body 110 in the top region thereof. The primed wound treatment composition storage and delivery container body 160 is secured to the wound treatment barrier body interior surface 113 of the wound treatment barrier body 110 by joining the dense hook segment 170 and the dense loop segment 172 to one another in accordance with a secure wound composition carrier to interior of support element step 920. It is understood that the secure wound composition carrier to interior of support element step 920 can be accomplished in a variety of alternative methods. In a first alternative arrangement, the dense loop segment 172 can be secured to the primed wound treatment composition storage and delivery container body attachment surface 164 and the dense hook segment 170 can be secured to the wound treatment barrier body interior surface 113 of the wound treatment barrier body 110. In another alternative arrangement, the primed wound treatment composition storage and delivery container body attachment surface 164 can be secured to the wound treatment barrier body interior surface 113 of the wound treatment barrier body 110 by an adhesive, such as a bonding agent, a double sided adhesive tape, and the like. In yet another alternative arrangement, the primed wound treatment composition storage and delivery container body attachment surface 164 can be secured to the wound treatment barrier body interior surface 113 of the wound treatment barrier body 110 by a mechanical fastener, such as a snap stud and socket, a hook and loop, a magnetized element and a ferrous or magnetic element, and the like. In one configuration, the primed wound treatment composition storage and delivery container body 160 can be fabricated of a ferrous or magnetic material and a magnetized element can be secured to the wound treatment barrier body interior surface 113 of the wound treatment barrier body 110. In the alternative arrangement, the primed wound treatment composition carrier 169 can be directly secured to the wound treatment barrier body interior surface 113 of the wound treatment barrier body 110 using any suitable attachment configuration, including a mechanical fastener, a bonding agent, and the like. A non-moisture impervious or non-wetting material can be applied to the attachment surface of the wound treatment composition carrier 168 to aid in the securing process.

The contactless wound treatment barrier assembly 100 is prepared for placement and attachment to the patient in accordance with a prepare support element for placement and attachment to the body at a location covering the wound step 922. When using the contactless wound treatment barrier assembly 100, (and similarly, the contactless wound treatment barrier assembly 300, 400), the support flange adhesive protective cover 122 is removed from the contactless wound treatment barrier assembly 100, exposing the support flange adhesive 120. When using the contactless wound treatment barrier assembly 200, the user would prepare the contactless wound treatment barrier elastic retention members 230. In alternative attachment solutions, the user would obtain and prepare the associated materials, such as tape, straps, and the like.

When the contactless wound treatment barrier assembly 100 and any other necessary materials is properly prepared, the contactless wound treatment barrier assembly 100 is placed onto the patient's exemplary skin 510 at a location covering the exemplary wound 512 in accordance with a place support element on the body at the location covering the wound step 924. Once properly positioned, the contactless wound treatment barrier assembly 100 is secured to the patient's exemplary skin 510 at the location covering the exemplary wound 512 in accordance with a secure support element to the body at the location covering the wound step 926.

Once secured, the wound treatment composition 167 is released from the wound treatment composition carrier 168 in a gaseous state within the interior volume 115 of the wound treatment barrier body 110 or any other chamber formed by any wound treatment barrier in accordance with a treat wound by gaseous application of wound treatment composition around the wound step 930.

The healing process is detailed as follows:

### Healing Phases:

Phase 1: Hemostasis, blood clotting occurs here
Phase 2: Inflammation, hypoxic state (low oxygen and high Nitric Oxide are needed here), activate genes needed for fighting infection
Phase 3: Proliferation of cells and new skin forming, normoxic state (high pressure oxygen helps healing)
Phase 4: Remodeling

The contactless wound treatment barrier assembly 100 can be removed from the exemplary injured region 500 on a periodic basis, such as daily in accordance with a remove support element from the body after a period of time step 940. If needed, the process is repeated to continue treatment in accordance with a repeat application process as needed step 950.

Treatment of the exemplary wound 512 can be discontinued once the healing process reaches a certain point. The medical professionals can provide guidance to the patient regarding the length of use of the contactless wound treatment barrier assembly 100.

Although the above is directed towards a treatment of a dermatologic wound, it is understood that the same apparatus and method can be adapted for treating sore muscles, fatigue, and the like. The distinction would be the selection of the wound treatment composition 167.

The following are tables comprising chemical compositions of various essential oils for use as alternative medicines. The essential oils can be utilized as the wound treatment composition 167.

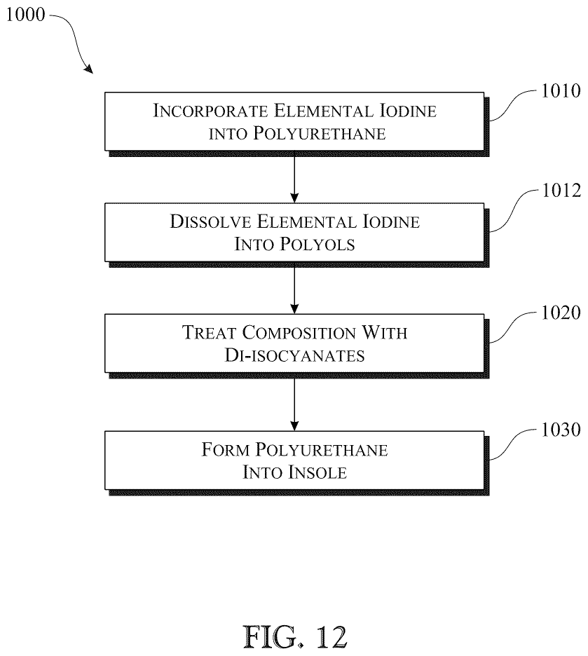

An exemplary elemental iodine impregnated insole fabrication process flow diagram 1000 is presented in FIG. 12. The elemental iodine impregnated insole fabrication process flow diagram 1000 initiates by incorporating elemental iodine into a liquid mixture to formulate a volume of polyurethane (step 1010). The two liquid components are mixed together to initiate the polymerization process. The process continues by dissolving elemental iodine into the liquid polyols (step 1012). The process illustrated in the elemental iodine impregnated insole fabrication process flow diagram 1000 continues where the iodine solution in polyols (step 1012) is treated with Di-isocyanates (step 1020). The mixture reacts to form a high volume foam (“Bun”) that is cut into sheets. The sheet of the polyurethane composition is cut into an insole using any suitable cutting process, including die cutting, laser cutting, computer controlled cutting or shearing, or any other suitable cutting process. Alternatively, the mixture comprising the iodine solution in polyols and treated with Di-isocyanates (as created in steps 1012 and 1020) can be poured into an insole mold (step 1030), eliminating the cutting step. The latter process makes a denser foam for longer term slow release of iodine gas. In one example, the polyurethane composition can be molded into the shape of the insole. In another example, the polyurethane composition can be rolled into a planar sheet and then cut to the shape of the insole using any suitable shaping process, such as later cutting, computer controlled cutting, sheering with a steel-rule die, and the like. Markings, such as sizing markings, use instructions, manufacturer's markings, materials identification, medical information, and the like, can be applied to the material at any suitable step of the process.

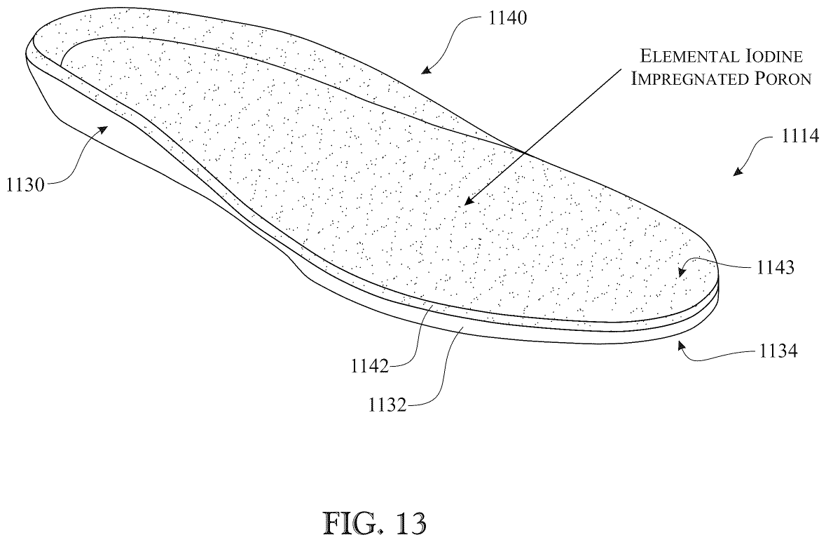

An exemplary wound treatment shoe insole 1114 is presented in FIG. 13. The wound treatment shoe insole 1114 comprises a wound treatment shoe insole core 1130 and a wound treatment shoe insole microporous polyurethane foam 1140. One exemplary microporous polyurethane foam is manufactured under a trade name PORON®. The microporous polyurethane foam has a property desirable for the subject process for treating a wound, where the microporous polyurethane foam absorbs and retains moisture while slowly releasing absorbed liquid composition in a gaseous format. The wound treatment shoe insole core 1130 is fabricated having a wound treatment shoe insole core body 1132 shaped to contour to a bottom surface of a patient's foot. The wound treatment shoe insole core body 1132 includes an insole core body bottom surface 1134 and an opposite top surface (an equivalent to the insole core body top surface 733 of the contactless wound treatment shoe insole core 730 illustrated in FIG. 8). The wound treatment shoe insole microporous polyurethane foam 1140 is fabricated of a microporous polyurethane foam material that creates a wound treatment shoe insole Microporous polyurethane foam body 1142, wherein the wound treatment shoe insole microporous polyurethane foam body 1142 is overmolded or laminated upon a wound treatment shoe insole core body 1132. A volume of wound treatment composition 167 is impregnated into the wound treatment shoe insole microporous polyurethane foam body 1142 at any reasonable point in a fabrication process. In one example, the wound treatment composition 167 can be mixed into the microporous polyurethane foam material prior to the overmolding process. In a second example, the wound treatment composition 167 can be mixed into the microporous polyurethane foam material during the overmolding process. In a third example, the wound treatment composition 167 can be applied to the microporous polyurethane foam material after formation of the wound treatment shoe insole 1114. In the third example, the wound treatment composition 167 can be applied to a limited area of the insole microporous polyurethane foam top surface 1143 or the entire surface of the insole microporous polyurethane foam top surface 1143.

One distinction of the wound treatment shoe insole 1114 over the previously described insoles 614, 714, 814 is that the wound treatment shoe insole core body 1132 is shaped to contour to a bottom of the patient's foot. The wound treatment shoe insole microporous polyurethane foam body 1142 would follow the contour of the wound treatment shoe insole core body 1132, as illustrated. It is noted that any of the previously described insoles 614, 714, 814 can include this feature.

As with the contactless wound treatment shoe insole 614, the contactless wound treatment shoe insole 1114 can also be used to reduce or eliminate malodorous odors, such as those associated with athlete's foot, wounds, and other malodorous odors. In an application directed to reduce or eliminate malodorous odors the contactless wound treatment shoe insole 1114 can more appropriately be referred to as a malodorous odor treatment shoe insole 1114 and the components can be referred to by a like adjective.

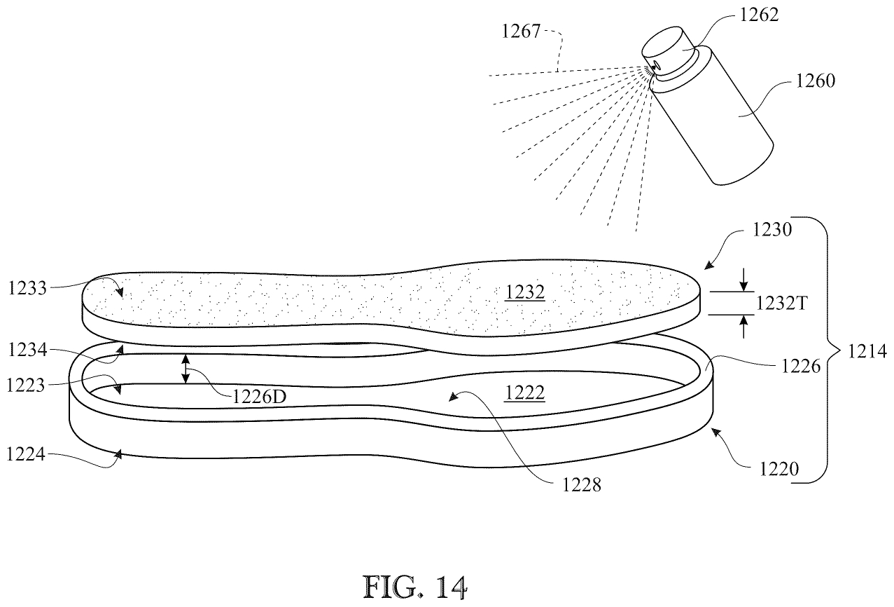

Each of the previously described insoles 614, 714, 814, 1114 were fabricated with the wound treatment composition 167 applied to the insole. An exemplary wound treatment shoe insole 1214 is introduced in FIG. 14. The exemplary wound treatment shoe insole 1214 can use a wound treatment shoe insole core 1230 as a single layer or in conjunction with a contactless wound treatment shoe insole spacing member 1220. The exemplary wound treatment shoe insole core 1230 is fabricated of a wound treatment shoe insole core body 1232 shaped to follow an interior peripheral edge of a shoe; the wound treatment shoe insole core body 1232 having an insole core body top surface 1233 and an insole core body bottom surface 1234.

A wound treatment shoe insole 1214 illustrates an alternative to the previously described insoles. The previously described insoles have a wound treatment composition 167 applied to the manufacturer. Conversely, the wound treatment shoe insole 1214 presents an exemplary insole where a wound treatment composition 1267 can be applied by a manufacturer, a doctor or other medical professional, a pharmacist, the patient, a caretaker, or any other suitable person. In the exemplary illustration presented in FIG. 14, the wound treatment composition 1267 is stored in a wound treatment composition storage and delivery container body 1260. The wound treatment composition storage and delivery container body 1260 includes a dispensing mechanism, such as a wound treatment composition delivery nozzle 1262. The wound treatment composition 1267 would be applied to the insole core body top surface 1233 and/or the insole core body bottom surface 1234 by any of by the manufacturer, the doctor or other medical professional, the pharmacist, the patient, the caretaker, or any other suitable person prior to use by the patient. The patient would obtain the wound treatment shoe insole 1214, apply the wound treatment composition 1267 to the wound treatment shoe insole core body 1232 (if the wound treatment composition 1267 was not previously applied) in accordance with instructions provided by the supplier of the wound treatment composition 1267, the doctor, the medical professional, the pharmacist, or any other authorized party, and insert the wound treatment shoe insole 1214 into a respective shoe. The composition of the wound treatment composition 1267 might require a time delay between application of the wound treatment composition 1267 to the wound treatment shoe insole core body 1232 and use of the wound treatment shoe insole 1214. Once the wound treatment shoe insole 1214 is properly prepared, the user would then place a sock on the respective foot and insert the foot into the shoe. It is understood that the wound treatment shoe insole 1214 can be fabricated having a plurality of layers laminated to one another as previously described. In an alternative application, the dry wound treatment insole 1214 can be inserted into the sock and the foot inserted into the insole containing sock. If the foot wound is too irritating and painful, the wound treatment insole 1214 can be loosely wrapped on a plantar area of the wounded foot with gauze or a bandage.

In another use, the wound treatment shoe insole 1214 can include the contactless wound treatment shoe insole spacing member 1220. The contactless wound treatment shoe insole spacing member 1220 includes an insole spacing member peripheral frame 1226 extending upward from a peripheral edge of a contactless wound treatment shoe insole spacing member base 1222. Orientation of the contactless wound treatment shoe insole spacing member base 1222 can be described as having an insole spacing member base top surface 1223 and an insole spacing member base bottom surface 1224. The insole spacing member peripheral frame 1226 is designed having an insole spacing member peripheral frame depth 1226D that is greater than a wound treatment shoe insole core body thickness 1232T. In use, the wound treatment shoe insole core body 1232 would be inserted into a cavity or an insole spacing member base interior volume 1228 defined by the insole spacing member base top surface 1223 and the insole spacing member peripheral frame 1226. The wound treatment shoe insole core body 1232 can be retained by friction between a peripheral edge of the wound treatment shoe insole core body 1232 against an interior surface of the insole spacing member peripheral frame 1226, by an adhesive placed between the insole spacing member base top surface 1223 and the insole core body bottom surface 1234, by a bonding agent placed between the insole spacing member base top surface 1223 and the insole core body bottom surface 1234, by a double sided adhesive tape placed between the insole spacing member base top surface 1223 and the insole core body bottom surface 1234, by a dense hook and loop tape placed between the insole spacing member base top surface 1223 and the insole core body bottom surface 1234, or by any other suitable retention element.

The insole spacing member peripheral frame 1226 would be of a size and shape to provide a gap between a bottom surface of a patient's foot and the insole core body top surface 1233 of the wound treatment shoe insole core 1230. The insole spacing member peripheral frame depth 1226D would be greater than the wound treatment shoe insole core body thickness 1232T. This provides another arrangement of a contactless wound treatment system. An upper edge of the insole spacing member peripheral frame 1226 would contact the bottom surface of a patient's foot. Since the insole spacing member peripheral frame depth 1226D is greater than the wound treatment shoe insole core body thickness 1232T, the insole spacing member peripheral frame 1226 would maintain a gap between the bottom surface of a patient's foot and the insole core body top surface 1233 of the wound treatment shoe insole core 1230. The combination would preferably be used when the patient is off their feet. This would ensure the insole spacing member peripheral frame 1226 provides sufficient support to maintain a gap between the bottom surface of a patient's foot and the insole core body top surface 1233.

Although the wound treatment shoe insole 1214 is described and shaped for use as an insole, the wound treatment shoe insole 1214 can be sized and shaped for use with any wound and would location. For example, the wound treatment shoe insole 1214 can be circular or oval in shape, creating an apparatus similar to the contactless wound treatment barrier assembly 100 or the contactless wound treatment barrier assembly 200 described above. In this arrangement, the wound treatment shoe insole 1214 can be secured using a wrap or any other suitable retention element.

As with the contactless wound treatment shoe insole 614, the contactless wound treatment shoe insole 1214 can also be used to reduce or eliminate malodorous odors, such as those associated with athlete's foot, wounds, and other malodorous odors. In an application directed to reduce or eliminate malodorous odors the contactless wound treatment shoe insole 1214 can more appropriately be referred to as a malodorous odor treatment shoe insole 1214 and the components can be referred to by a like adjective.

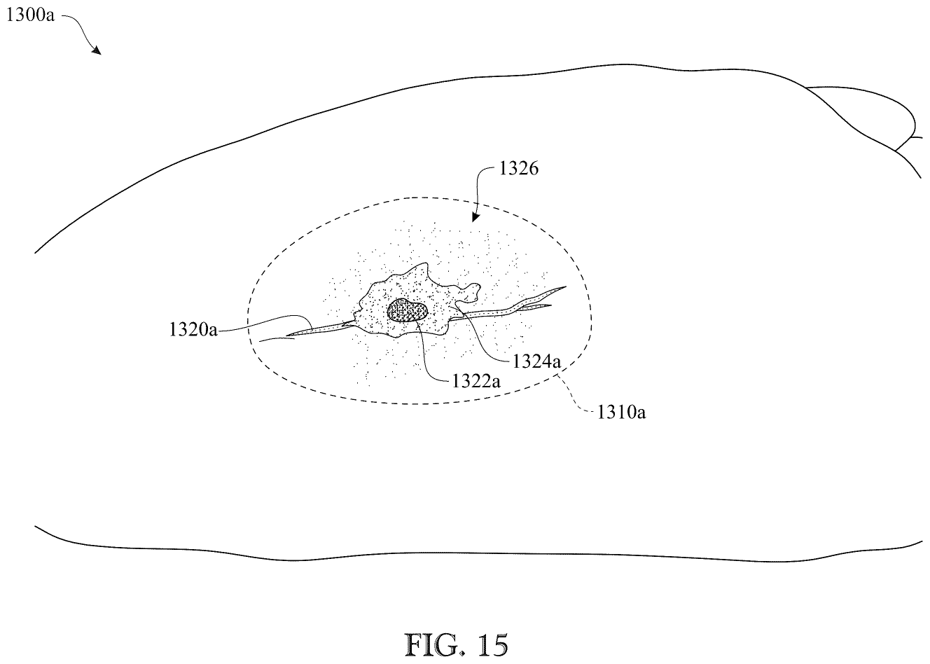

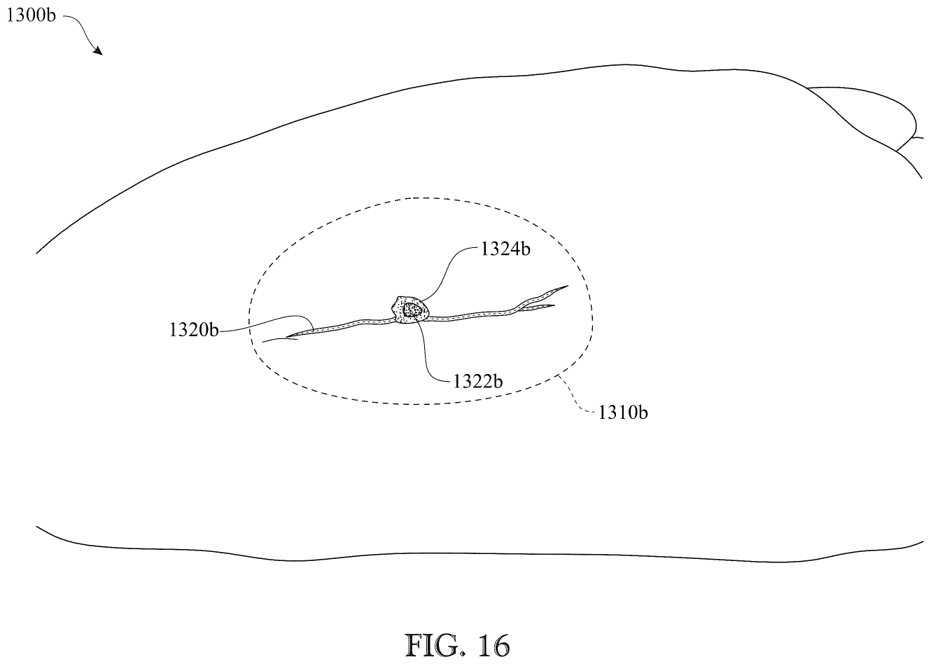

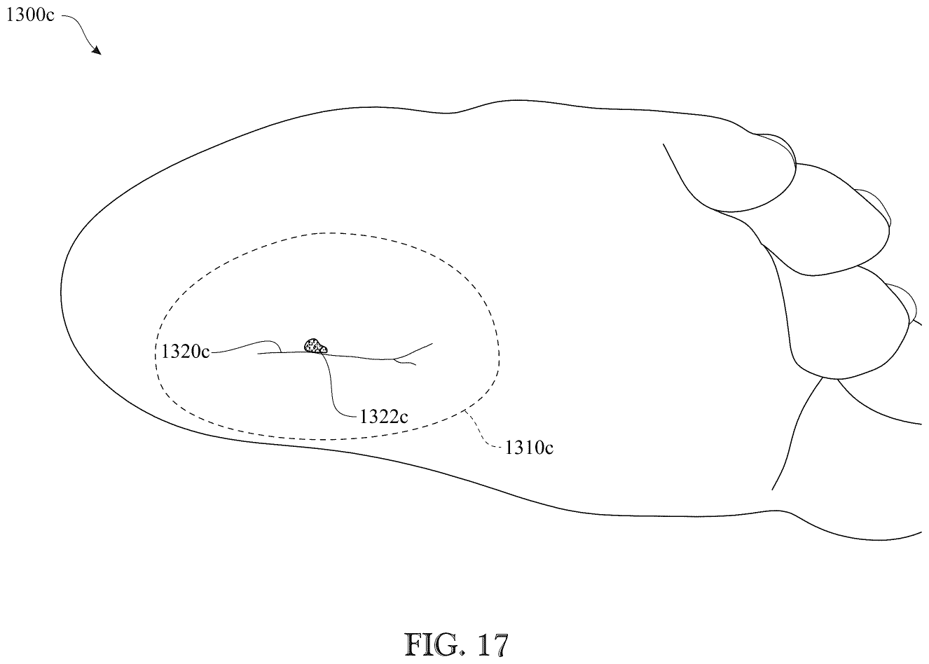

One primary focus of the present invention is to treat a wound on a patient's foot treatment (after one (1) week 1300a), (after two (2) weeks 1300b), (after three (3) weeks 1300c). It is common for wounds, such as the wounded 1310a, to take many weeks or months to heal, particularly if the patient has underlying medical conditions known to inhibit healing of wounds, such as diabetes, poor blood circulation, or any other medical condition that inhibits healing. Progression of a wound during treatment (after one (1) week 1310a), (after two (2) weeks 1310b), (after three (3) weeks 1310c) healing in conjunction with the exemplary treatment is presented in FIGS. 15 through 17. The wounded foot after one (1) week of treatment 1300a is presented in FIG. 15. The wounded foot after two (2) weeks of treatment 1300b is presented in FIG. 16. The healed foot after three (3) weeks of treatment 1300c is presented in FIG. 17. Progression of the healing process of the wound (after one (1) week 1310a), (after two (2) weeks 1310b), (after three (3) weeks 1310c) is identified by a suffix, where reference characters comprising the suffix “a” refers to the wound after one (1) week of treatment; where reference characters comprising the suffix “b” refers to the wound after two (2) weeks of treatment; and where reference characters comprising the suffix “c” refers to the wound after three (3) weeks of treatment. The exemplary wound 1310a, 1310b, 1310c includes a laceration 1320a, 1320b, 1320c; a deep wound 1322a, 1322b, 1322c and a shallow wound 1324a, 1324b.

Each of the above are designed to accomplish the same task: treating a patient's wound by providing a wound treatment composition carrier 169, 369, 469, 668, 669, 732, 832, 1232, wherein the wound treatment composition carrier 169, 369, 469, 668, 669, 732, 832, 1232 is carrying a wound treatment composition 167, 867, 1267. The wound treatment composition 167, 867, 1267 is retained in the wound treatment composition carrier 169, 369, 469, 668, 669, 732, 832, 1232 in at least one of a solid state and a liquid state. The wound treatment composition carrier 169, 369, 469, 668, 669, 732, 832, 1232 can be inserted into a cavity or interior volume 115, 315, 415, (628, 638), (629, 639), 1228 created by an environmental barrier 110, 210, 310, 410, 628, 629, 1220. The environmental barrier 110, 210, 310, 410, 628, 629, 1220 can be provided in any suitable shape, including a dome, a cone, a truncated cone, a hemisphere, a cylinder, a cube, a cuboid, a pyramid, a tetrahedron, a square pyramid, a hexagonal pyramid, a truncated pyramid, an outline of an insole, or any other suitable shape. The wound treatment composition carrier 169, 369, 469, 668, 669, 732, 832, 1232 is positioned proximate a patient's wound 512. The patient's wound 512 is treated by exposing the patient's wound 512 to the out-gassed wound treatment composition 167, 867, 1267 over a period of time. The patient's wound 512 would remain completely exposed (lacking any covering) during the exposure to the out-gassed wound treatment composition 167, 867, 1267. Covering the patient's wound 512 would inhibit the exposure to the out-gassed wound treatment composition 167, 867, 1267. The covering can additionally collect condensed out-gassed wound treatment composition 167, 867, 1267 (iodine), exposing the skin 510 of the patient 500 to undesirable direct contact with the condensed wound treatment composition 167, 867, 1267 (iodine). This is applicable to all of the embodiments described herein.

The wound treatment composition 167, 867, 1267 can be any of the above disclosed compositions. Some preferred wound treatment compositions 167, 867, 1267 include an antimicrobial compound, iodine, elemental iodine, and an iodine releasing compound.

The wound treatment composition carrier 732, 832 can be provided as a series of layers. In one arrangement, a top layer 742, 842 can be provided over the wound treatment composition carrier 732, 832, wherein the top layer 742, 842 would be exclusive of any wound treatment composition 167, 867. In a second arrangement, the wound treatment composition can be carried by wound treatment composition carrier inserts 668, 669, where the wound treatment composition carrier inserts 668, 669 would be seated within a first layer 620. A top layer 630 would be provided over a top surface of the first layer 620. Apertures through the top layer 630 define the cavity and spatial arrangement between the patient's wound and the wound treatment composition carrier inserts (carriers) 668, 669. The environmental barrier 110, 210, 310, 410, 628, 629, 1220 can include features to enable drawing the gaseous state of the wound treatment composition 167, 867, 1267 from the wound treatment composition carrier 169, 369, 469, 668, 669, 732, 832, 1232 by introducing at least one of a pressure within the cavity and a vacuum within the cavity. This can be accomplished by applying and removing pressure from the barrier, thus increase and decreasing the pressure within the cavity 115, 315, 415, (628, 638), (629, 639), 1228 defined by the environmental barrier 110, 210, 310, 410, 628, 629, 1220.

The wounded foot after one (1) week of treatment 1300a is presented in FIG. 15. After one (1) week, the laceration portion of the wound after one (1) week of treatment 1320a shows the dehiscence (separation) starting to reduce in size. Similarly, the deeper portion of the wound area after one (1) week of treatment 1322a also shows a reduction in the dehiscence (reduces in diameter). Following suit, the shallower portion of wound surrounding the deeper wound after one (1) week of treatment 1324a also shows a reduction in the dehiscence (reduces in diameter). During the first week of treatment, an Iodine stain on stratum corneum surrounding the wound area after one (1) week of treatment 1326 can be formed, leaving a visible stain. The Iodine stain disappears over time, as illustrated in FIGS. 16 and 17.

The wounded foot after two (2) weeks of treatment 1300b is presented in FIG. 16. After two (2) weeks, the laceration portion of the wound nearly or completely healed after two (2) weeks of treatment 1320b shows a continuation in a reduction in the dehiscence (separation). At this point, the laceration portion of the wound nearly or completely healed after two (2) weeks of treatment 1320b is considered to be completely healed. Similarly, the deeper portion of the wound after two (2) weeks of treatment 1322b also shows a continued reduction in the dehiscence (further reduction in the diameter). A scab (fibrin plug) forms covering the deeper portion of the wound after two (2) weeks of treatment 1322b. Following suit, the shallower portion of the wound after two (2) weeks of treatment 1324b shows a significant reduction in the dehiscence (significant reduction or more than 80% reduction in the diameter). During the second week of treatment, the Iodine stain on stratum corneum surrounding the wound area after one (1) week of treatment 1326 is significantly reduced or essentially gone.

The healed foot after three (3) weeks of treatment 1300c is presented in FIG. 17. After three (3) weeks, the keratin has fallen out and the skin is smooth and pink. The wound after three (3) weeks of treatment 1310c is essentially healed. The laceration portion of the wound after three (3) weeks of treatment 1320c shows a significant reduction in the dehiscence (separation), where the laceration has disappeared by more than 95%. The deeper portion of the wound after three (3) weeks of treatment 1322c also shows a significant reduction in the dehiscence (significant reduction in the diameter). The scab covering the deeper portion of the wound after three (3) weeks of treatment 1322c is minimal and nearing, beginning to, or already falling off. At this point during the treatment, the shallower portion of the wound after two (2) weeks of treatment 1324b has completely healed. During the third (3) week of treatment, the Iodine stain on stratum corneum surrounding the wound area after one (1) week of treatment 1326 has completely disappeared.

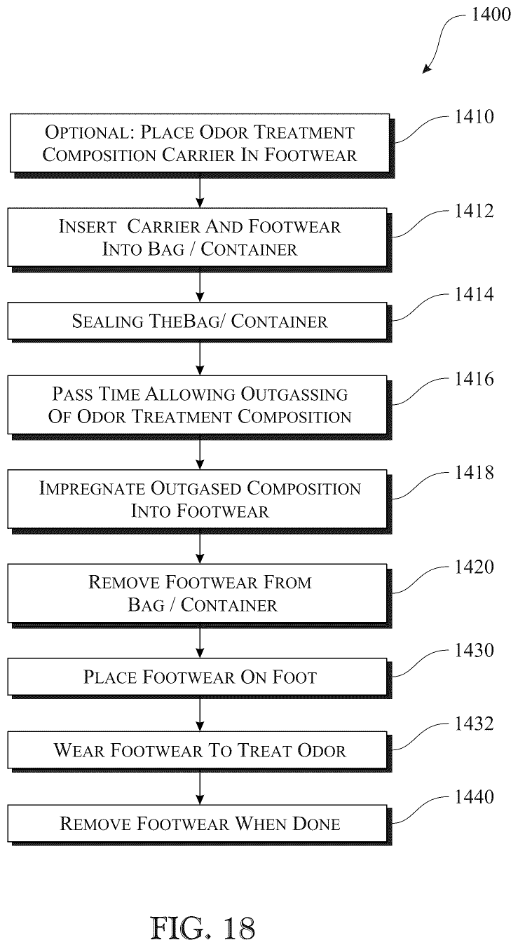

A treatment of a wound and/or a malodorous odor can be accomplished using any of the treatment carriers described above. Although described as wound treatment composition carriers, the treatment carriers described above can also be described as odor treatment composition carriers. In yet another process, the treatment can be accomplished by transferring the treatment composition to a garment or other worn object, as described in an exemplary odor treatment process flow diagram 1400 presented in FIG. 18. Using the illustration in FIG. 7 being adapted towards an odor treatment apparatus used to treat Athlete's foot or other like malodorous odor causing conditions as a reference, the exemplary odor treatment process flow diagram 1400 initiates with an optional step of placing an odor treatment composition carrier 614 into a footwear 600 (block 1410). The odor treatment composition carrier 614 and the footwear 600 are inserted into a bag or other air impervious container (block 1412). The bag or other air impervious container is sealed (block 1414) creating the air impervious environment containing the odor treatment composition carrier 614 and the footwear 600. This allows the odor treatment composition (represented by 668, 669) to be emitted, over time, from the solid state or liquid state in a gaseous state (block 1416), thus embedding the odor treatment composition (represented by 668, 669) in the footwear 600 (block 1418). Once the odor treatment composition (represented by 668, 669) is sufficiently embedded within the footwear 600, the footwear 600 is removed from the bag/enclosure (block 1420). The time required can vary based upon the make-up of the odor treatment composition (represented by 668, 669), a rate of change in state, a volume within the enclosure, a desired quantity of the odor treatment composition (represented by 668, 669) to be embedded within the footwear 600, and the like. It is desirous to remove the footwear 600 from the enclosure shortly prior to use to optimize the effectiveness of the now embedded odor treatment composition. The footwear 600 would be placed upon a foot of a patient (block 1430) to be worn over a period of time to treat the source of the malodorous odor (block 1432). When done wearing the footwear 600, the footwear 600 is removed from the foot of the patient (block 1440). The removed footwear 600 can be reinserted into the enclosure along with the odor treatment composition (represented by 668, 669) (return to block 1412).

The details in the odor treatment process flow diagram 1400 are only exemplary and the odor treatment process flow diagram 1400 can be applied to alternatives, with several examples being described herein. For example, the footwear 600 can alternatively be a shirt, shorts, pants, a bandage, or any other wearable article that would be placed upon a patient near a location carrying a source of the malodorous odor. The odor treatment composition carrier 614 can be formed in a shape of an insole or any other form factor disclosed herein, a sphere, an egg, a prolate spheroid (football shaped), a cube, a block, an insert for short, an insert for pants, an insert for a shirt, or any other suitable shape. Examples of locations of sources of malodorous odor can include a foot, underarms (armpits), groin or private area, an area proximate a sore or infection, etc. The above listed wearable articles can be worn covering the area carrying the source of the malodorous odor. For example, a bandage can be wrapped over a wound emitting the malodorous odor. The same process can be applied to treatment of a wound or both a wound and a malodorous odor.

The contactless wound treatment barrier assembly 100 and the contactless wound treatment barrier assembly 300 each describe an apparatus having a primed wound treatment composition storage and delivery container assembly 150, 350 carried within an interior of an encapsulating structure. This requires removal of the contactless wound treatment barrier assembly 100, 300 from the exemplary skin 510 in order to manage the application of the wound treatment composition. Typically, once the contactless wound treatment barrier assembly 100, 300 is removed, the contactless wound treatment barrier assembly 100, 300 must be replaced. The contactless wound treatment barrier assembly 100, 300 is commonly adhered to the patient using an adhesive. Removal of the adhesively retained contactless wound treatment barrier assembly 100, 300 can cause irritation or injury to the exemplary skin 510 of the patient. The irritation or injury can aid in spreading infections caused by any microorganisms (such as bacteria, archaea, protozoa, algae, fungi, viruses, and multicellular animal parasites (helminths), and the like), with the concerns being towards those which causes disease.

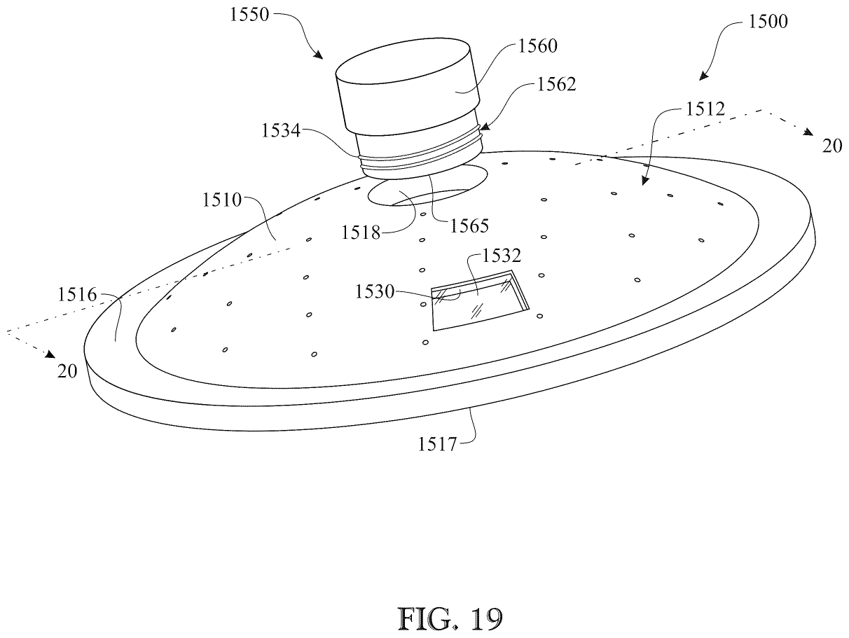

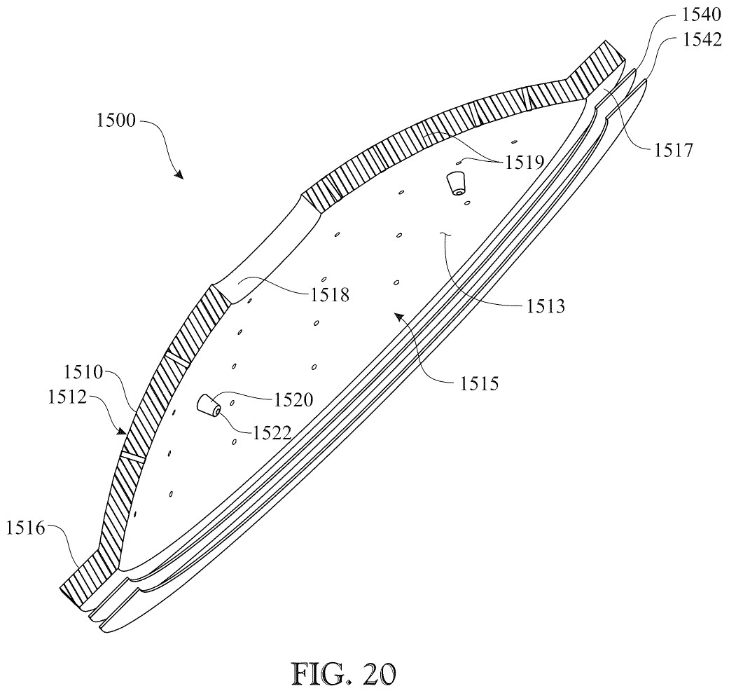

A contactless wound treatment barrier assembly 1500 introduces an apparatus having a wound treatment barrier body wound composition container body receiving aperture 1518 passing through a wound treatment barrier body 1510 of the contactless wound treatment barrier assembly 1500, as illustrated in FIGS. 19 through 23. The contactless wound treatment barrier assembly 1500 and the contactless wound treatment barrier assembly 300 include a number of like elements. Like elements of the contactless wound treatment barrier assembly 1500 and the contactless wound treatment barrier assembly 300 are numbered the same, whereby the elements of the contactless wound treatment barrier assembly 1500 are preceded by the numeral “15”. The wound treatment barrier body wound composition container body receiving aperture 1518 is sized to receive and retain a wound treatment composition storage and delivery container assembly 1550. The wound treatment composition storage and delivery container assembly 1550 can optionally includes one or more wound treatment composition storage and delivery container body securing features 1564 at least partially circumscribing a wound treatment composition storage and delivery container body exterior surface 1562 of a wound treatment composition storage and delivery container body 1560. The exemplary wound treatment composition storage and delivery container assembly 1550 includes a step down lower section creating a seating surface between the threaded section and an upper section. Essentially, the lower section has a smaller diameter than the upper section. The demarcation between the lower section and the upper section provides a stop when inserting the wound treatment composition storage and delivery container assembly 1550 into the wound treatment barrier body wound composition container body receiving aperture 1518.

A method of using the contactless wound treatment barrier assembly 1500 would be similar to the methods of using the contactless wound treatment barrier assembly 100, the contactless wound treatment barrier assembly 200, and contactless wound treatment barrier assembly 300 as previously described herein. In use, the wound treatment composition carrier 1569 would either reside within or be inserted into an interior of the wound treatment composition storage and delivery container body 1560, the interior being defined by a wound treatment composition storage and delivery container body interior surface 1563 of the wound treatment composition storage and delivery container body 1560. The wound treatment composition carrier 1569 can be a sponge or sponge like material. The wound treatment composition carrier 1560 can be fabricated of any suitable material having properties capable of absorbing a composition in a liquid state or carrying the composition in a solid state and emitting the composition in a gaseous state. Examples of suitable material can be a porous material, such as an open cell foam, an open cell polyurethane, a closed cell polyurethane, a reticulated polyurethane foam, silicone, rubber, silicone-rubber, a sponge; an absorbent material, such as a fabric, a carpet, and the like.

The wound treatment composition carrier 1569 would be impregnated with the wound treatment composition. The wound treatment composition storage and delivery container assembly 1550 can optionally include a cover that would be threadably assembled to the lower portion of the wound treatment composition storage and delivery container body 1560. When a cover is included, the cover would be removed prior to insertion. The lower portion of the wound treatment composition storage and delivery container body 1560 would be inserted through the wound treatment barrier body wound composition container body receiving aperture 1518. The wound treatment barrier body 1510 would preferably be fabricated of a pliant material, such as Ethylene-Vinyl Acetate Copolymer (EVA foam) or Poly Ethylene-Vinyl Acetate Copolymer (PEVA). The EVA foam is a non-irritant to the patient and is iodine non-absorbent. The pliancy of the wound treatment barrier body 1510 aids in securing the wound treatment composition storage and delivery container assembly 1550 within the wound treatment barrier body wound composition container body receiving aperture 1518 and creating a gaseous seal there between. A plurality of wound treatment barrier body perforation 1519 can be created through the encapsulating portion of the wound treatment barrier body 1510. The wound treatment barrier body perforations 1519 provide a means for replenishing Oxygen while retaining the wound treatment composition within the interior of the wound treatment barrier body 1510. The wound treatment barrier body perforations 1519 are preferably sized to enable an influx of Oxygen while deterring an outflow of the wound treatment composition, such as the gaseous iodine. Oxygen is a critical component in the healing process. The wound treatment barrier body perforations 1519 enable the influx of Oxygen to supply the exemplary wound 512 with the necessary Oxygen. In a condition where the wound treatment barrier body 1510 is impermeable, the impermeable material of the wound treatment barrier body 1510 will retain too much moisture, wherein the excessive collected moisture will macerate the patient's wound 512.

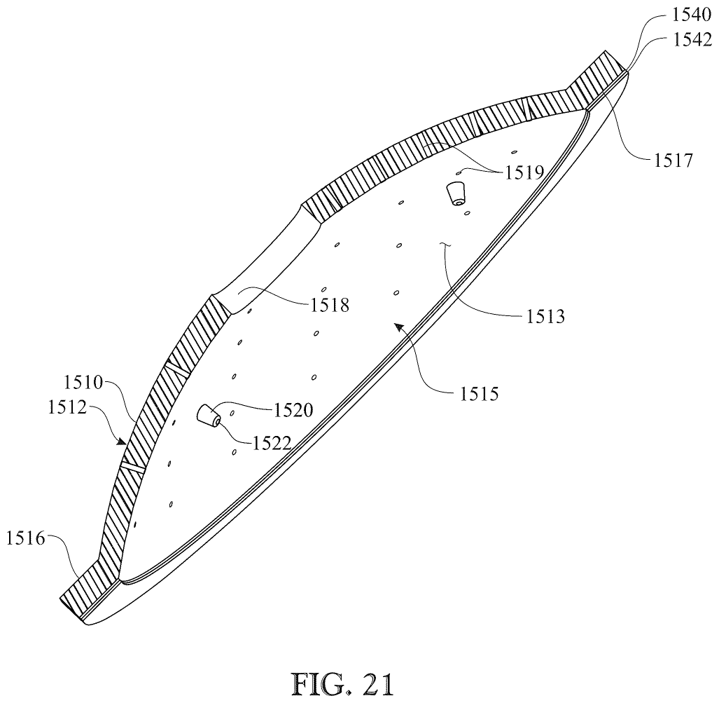

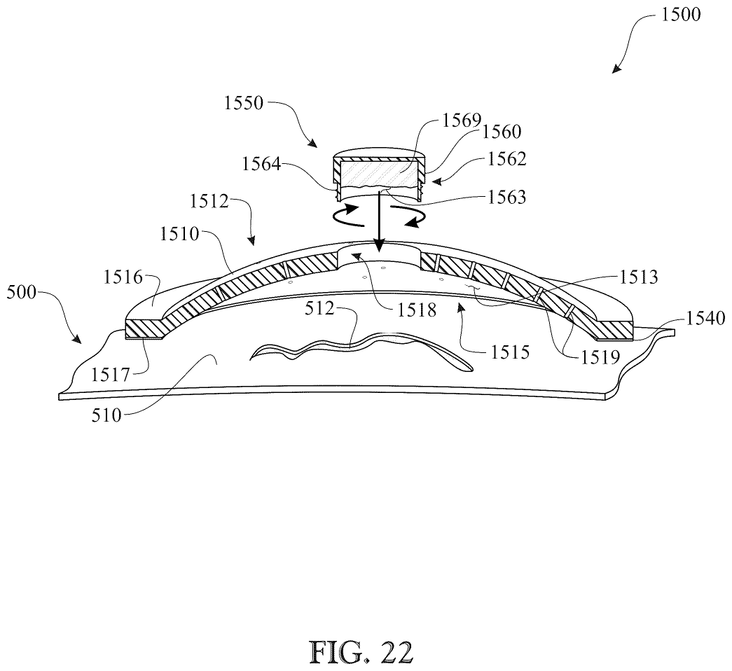

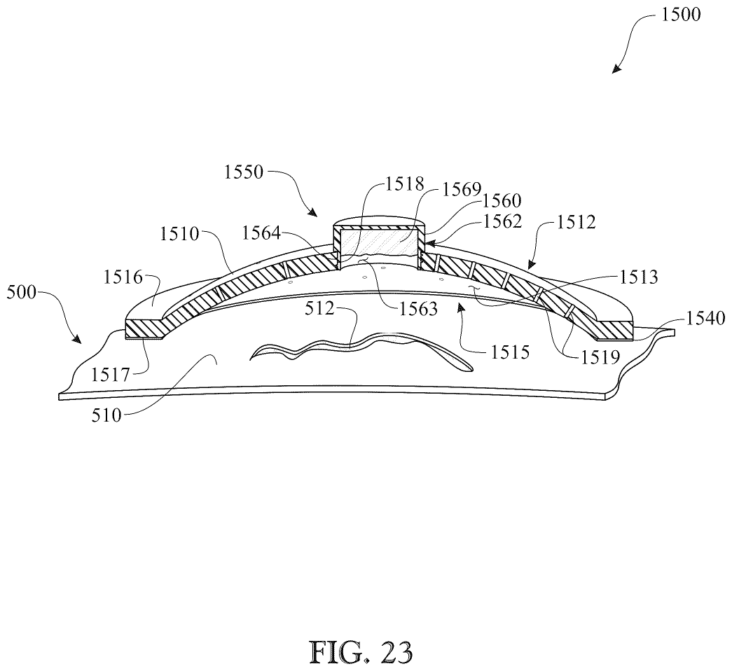

A wound treatment barrier body support double sided adhesive 1540 is secured to a wound treatment barrier body support flange contact surface 1517 of the wound treatment barrier body support flange 1516 of the contactless wound treatment barrier assembly 1500. A wound treatment barrier body support double sided adhesive protectant 1542 is provided on an exterior surface of the wound treatment barrier body support double sided adhesive 1540 to protect the adhesive of an exposed surface of the wound treatment barrier body support double sided adhesive 1540 until use, as illustrated in FIG. 21. The wound treatment barrier body support double sided adhesive protestant 1542 is carefully removed from the wound treatment barrier body support double sided adhesive 1540, exposing the adhesive. The wound treatment barrier body support double sided adhesive 1540 secures the contactless wound treatment barrier assembly 1500 to the exemplary skin 510 of the patient, as illustrated in FIGS. 22 and 23.

The wound treatment composition storage and delivery container assembly 1550 may be sealed by a primed wound treatment composition storage and delivery container closure similar to the closure 365. The closure can include threads that engage with the wound treatment composition storage and delivery container body securing feature 1564. When used, the closure would be removed from the wound treatment composition storage and delivery container assembly 1550, exposing the wound treatment composition carrier 1569.

The wound treatment composition storage and delivery container assembly 1550 with the exposed wound treatment composition carrier 1569 can be inserted into the wound treatment barrier body wound composition container body receiving aperture 1518 either before or after the step of securing of the contactless wound treatment barrier assembly 1500 to the exemplary skin 510. In an arrangement where the wound treatment composition storage and delivery container body 1560 includes a wound treatment composition storage and delivery container body securing feature 1564, the wound treatment composition storage and delivery container assembly 1550 would be rotated during the process of inserting the lower portion of the wound treatment composition storage and delivery container body 1560 through the wound treatment barrier body wound composition container body receiving aperture 1518, as illustrated in FIGS. 22 and 23.

Once the contactless wound treatment barrier assembly 1500 is applied to the patient and the wound treatment composition storage and delivery container assembly 1550 is inserted therein, the wound treatment composition outgases, providing a treatment environment where the wound treatment composition in a gaseous state treats the wound as described above.

The contactless wound treatment barrier assembly 1500 introduces several features enabling inspection of the exemplary wound 512 without removal of the contactless wound treatment barrier assembly 1500 from the patient. A first exemplary inspection feature is the ability to remove the wound treatment composition storage and delivery container assembly 1550 from the wound treatment barrier body 1510. Removal of the wound treatment composition storage and delivery container assembly 1550 from the wound treatment barrier body 1510 converts the wound treatment barrier body wound composition container body receiving aperture 1518 into a viewing port for inspection of the exemplary wound 512. A second exemplary inspection feature is a wound treatment barrier body viewing port 1530 formed through the wound treatment barrier body 1510, as presented in FIGS. 19 through 23. A wound treatment barrier body viewing port clear seal 1532 is preferably assembled to the wound treatment barrier body 1510 to seal the wound treatment barrier body viewing port 1530. Although the exemplary illustration presents a single wound treatment barrier body viewing port 1530, the wound treatment barrier body 1510 can include multiple wound treatment barrier body viewing ports 1530 spatially arranged thereabout. The wound treatment barrier body viewing port 1530 enables a medical professional an ability to inspect the exemplary wound 512 without removal of the contactless wound treatment barrier assembly 1500 or even the wound treatment composition storage and delivery container assembly 1550 from the contactless wound treatment barrier assembly 1500. This feature minimizes any interruption or impact on the treatment of the exemplary wound 512 of the patient. The inclusion of the wound treatment barrier body viewing port 1530 enables the medical professional the ability to determine if the wound treatment composition carrier 1569 needs to be changed and/or modified. The wound treatment barrier body viewing port 1530 can be included in any of the variants described herein.

One or more diffusers/heaters 1520 can be carried by the wound treatment barrier body interior surface 1513. Operational elements associated with the one or more diffusers/heaters 1520 can be arranged externally of the wound treatment barrier body 1510. Operational elements can include electrical power, compressed air, or any other supporting element. The diffusers/heaters 1520 can be used for any of a number of functions, including introducing airflow to circulate the wound treatment composition, increasing a temperature within the interior of the wound treatment barrier body 1510, each of the one or more diffusers/heaters 1520 preferably includes a diffuser/heater nozzle 1522. The diffuser/heater nozzle 1522 enables discharging of airflow, heated airflow, any chemicals, medication, control humidity within the interior of the wound treatment barrier body 1510, and the like. In another application, a vacuum can be applied to one or more diffusers/heaters 1520 to draw gas and/or fluid from the interior of the wound treatment barrier body 1510. The contactless wound treatment barrier assembly 1500 can additionally/alternatively include one or more sensors to monitor a volume of medication, chemicals, pH levels, and/or any other characteristic within the interior of the wound treatment barrier body 1510. The diffuser 1520 can act as an air circulating device.

The wound treatment composition storage and delivery container assembly 1550 can include a feature allowing adjustments to the volume of dispensed medication. In one example, the wound treatment composition storage and delivery container assembly 1550 can include a perforated rotating member that rotates about a second, fixed perforated member. The overlap between perforations adjusts a size of an effective opening, wherein the effective opening determines the volume of dispensed material. The process can be manually operated or automated. The automated process can employ sensors to determine the currently dispensed volume of medication and a logic circuit can determine if the dispensed volume needs to be increased or decreased, then adjusting the dispensing system accordingly. Although the disclosure describes one method of adjusting the dispensed volume, it is understood that any method of adjusting the dispensing can be utilized.

The design of the contactless wound treatment barrier assembly 1500 enables several key factors. The wound treatment barrier body viewing port 1530 enables monitoring of the condition and progress of the healing of the exemplary wound 512. The wound treatment barrier body viewing port 1530 also enables monitoring of the impact of the dosage or exposure of the exemplary wound 512 to the wound treatment composition. When the exposure of the wound treatment composition is determined to be excessive, the wound treatment composition storage and delivery container assembly 1550 is removed and resealed. A cap (not shown) is optionally inserted into the wound treatment barrier body wound composition container body receiving aperture 1518, sealing the interior volume 1515 of the contactless wound treatment barrier assembly 1500. The wound treatment composition storage and delivery container assembly 1550 can be uncovered and reinserted into the wound treatment barrier body wound composition container body receiving aperture 1518 following a gap in time. The gap in time can be determined based upon visual monitoring of the condition of the exemplary wound 512, a predetermined time span, or any other suitable basis. This cycling process avoids excessive exposure of the exemplary wound 512 to the wound treatment composition. The configuration of the contactless wound treatment barrier assembly 1500 permits insertion and removal of the wound treatment composition storage and delivery container assembly 1550 from the wound treatment barrier body wound composition container body receiving aperture 1518 enabling management of the exposure of the exemplary wound 512 to the wound treatment composition.

In one case study: A 12 cm diameter dome 1510 (2.5 cm at apex), with the opened Iodine-carrying-container 1550, was affixed around an ulcer 512 using medical tape across the wound treatment barrier body support flange 1516. This exemplary case study utilized domes 1510 lacking any perforations 1519. The placement of the dome 1510 ensured that the base of the dome 1510 did not touch the ulcer 512. After four (4) hours of treatment (exposure to the wound treatment composition), the Iodine delivery device 1550 was removed from the aperture 1518 on the dome 1510 and the aperture 1518 was covered with porous sterile gauze material for three (3) hours. The use of the porous sterile gauze material allowed the wound 512 to “breathe” Oxygen. After the three (3) hours of exposure to oxygen, the Iodine delivering device 1550 was reinserted into the opening 1518 of the dome 1510. The process was repeated for twenty-four (24) hours. After twelve (12) hours, the malador of the ulcer 512 had ceased, and the wound 512 was visibly drying up. At day two (2), the exposure of the wound 512 to the wound treatment composition (Iodine) using the opened Iodine-carrying-container 1550 was extended to six (6) hours; then the same three (3) hour exposure to Oxygen for the “breathing” cycle. Within one (1) week, the ulcers 512 developed a dry scab and the ulcers 512 were shrinking. After four (4) weeks, the ulcers 512 were completely healed, and only epithelization to rebuild new skin continued for additional 2-3 months. The dome 1510 was used on three (3) additional volunteering patients resulting in a 100% healing rate.

In a second case study, the 12 cm diameter dome 1510 (2.5 cm at apex) included multiple wound treatment barrier body perforations 1519, as illustrated herein. In this arrangement, the inclusion of the multiple wound treatment barrier body perforations 1519 allowed for a dilution of the wound treatment composition (iodine gas) by outflow diffusion while Oxygen can diffuse into the interior volume 1515 at the same time. This arrangement minimized any irritation of the wound 512 or adjacent skin 510 from exposure to the wound treatment composition (iodine gas).

In an arrangement where the patient's wound 512 were covered, the wound treatment composition (iodine) would condensate upon the covering thus placing the patient's wound 512 in an undesirable and potentially harmful condition of exposing the patient's wound 512 to direct contact with the iodine. The wound dressing placed upon the skin can additionally apply an excessive pressure upon the skin 510.

No need to remove the iodine delivery device for the porous dome, a second embodiment of the present invention, as the pores on the dome allow for a dilution of the iodine gas by outflow diffusion while oxygen can diffuse in at the same time. So, any irritation is kept to a minimum.

The wound treatment composition storage and delivery container assembly 1550 can additionally include a flow rate control mechanism for adjusting release of the wound treatment composition in the gaseous state, wherein the flow rate control mechanism is adjusted using at least one of a manual process and an automated process. The flow rate control mechanism preferably employs a pair of planar members, including a first planar member and a second planar member. Each of the first planar member and the second planar member include apertures passing therethrough. The apertures would be sized and arranged where, in a first rotated position, the first planar member and the second planar member create a complete seal and in a second rotated position, the first planar member and the second planar member create a complete seal, the apertures of the first planar member and the apertures of the second planar member are in alignment with one another maximizing an opening or passage for dispensing of the wound treatment composition in the gaseous state. The volume of dispensing wound treatment composition can be controlled by partially rotating one of the first planar member and the second planar member creating a partially (smaller) sized opening limited by a portion of the apertures of the first planar member and a portion of the apertures of the second planar member. This arrangement is analogous to a spice dispenser, such as the TABLECRAFT HGJ3RT Spice Shaker with Adjustable Lid.

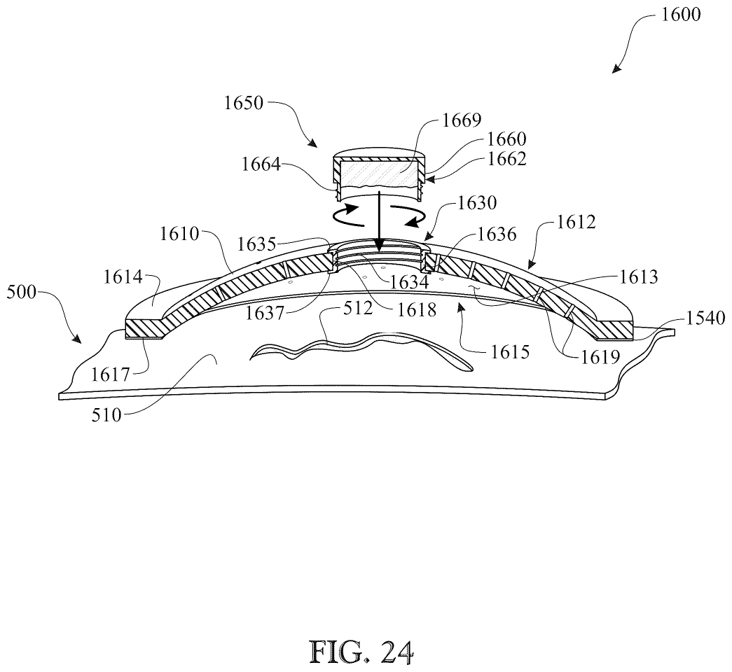

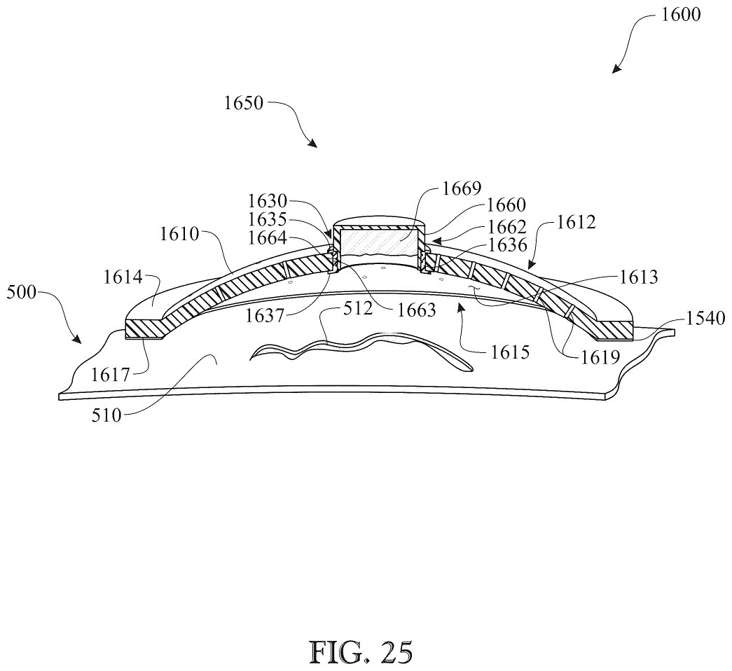

The assembly of the wound treatment composition storage and delivery container assembly 1550 to the contactless wound treatment barrier assembly 1500 relies upon the properties of the material used to fabricate the wound treatment barrier body 1510. A contactless wound treatment barrier assembly 1600 is a modified variant of the contactless wound treatment barrier assembly 1500, wherein the contactless wound treatment barrier assembly 1600 introduces a wound treatment composition container body receiving member 1630, wherein the wound treatment composition storage and delivery container assembly 1650 is assembled to the wound treatment composition container body receiving member exterior flange 1635, as illustrated in FIGS. 24 and 25. A majority of the elements of the contactless wound treatment barrier assembly 1600 and the contactless wound treatment barrier assembly 1500 are alike. Like elements of the contactless wound treatment barrier assembly 1600 and the contactless wound treatment barrier assembly 1500 are numbered the same, whereby the elements of the contactless wound treatment barrier assembly 1600 are preceded by the numeral “16”.

The exemplary wound treatment composition container body receiving member 1630 includes a wound treatment composition container body receiving member exterior flange 1635 extending radially outwardly from an outer edge of a wound treatment composition container body receiving member annular ring 1636 and a wound treatment composition container body receiving member interior flange 1637 extending radially outwardly from an inner edge of the wound treatment composition container body receiving member annular ring 1636 forming an outwardly shaped “C”. The edge of the wound treatment barrier body wound composition container body receiving aperture 1618 is inserted about a ring of the wound treatment composition container body receiving member annular ring 1636 extending between facing surfaces of the wound treatment composition container body receiving member exterior flange 1635 and the wound treatment composition container body receiving member interior flange 1637. A wound treatment composition container body receiving member threading 1634 is formed on an interior surface of the wound treatment composition container body receiving member annular ring 1636. The wound treatment composition container body receiving member threading 1634 is designed to matingly engage with a wound treatment composition storage and delivery container body securing feature 1664 of the wound treatment composition storage and delivery container assembly 1650.

Use of the contactless wound treatment barrier assembly 1600 mimics the use of the contactless wound treatment barrier assembly 1500 with the only modification is the wound treatment composition storage and delivery container assembly 1650 is assembled to the wound treatment barrier body 1610 by threadably assembling the wound treatment composition storage and delivery container body securing feature 1664 of the wound treatment composition storage and delivery container body 1660 and the wound treatment composition container body receiving member threading 1634 of the wound treatment composition container body receiving member 1630 to one another.

The wound treatment composition storage and delivery container assembly 1550, as described above, includes a wound treatment composition storage and delivery container body securing feature 1564 for use in joining the wound treatment composition storage and delivery container assembly 1550 to the wound treatment barrier body 1510. The contactless wound treatment barrier assembly 1600 employs the wound treatment composition container body receiving member 1630 as a member enabling threadably assembly of the wound treatment composition storage and delivery container assembly 1650 to the wound treatment barrier body 1610. The wound treatment composition container body receiving member 1630 is retained within the wound treatment barrier body wound composition container body receiving aperture 1618 by the wound treatment composition container body receiving member exterior flange 1635 and the wound treatment composition container body receiving member interior flange 1637. In a modified version of the wound treatment composition storage and delivery container assembly 1550, the same arrangement of the wound treatment composition container body receiving member 1630 can be adapted to the wound treatment composition storage and delivery container assembly 1550, replacing the wound treatment composition storage and delivery container body securing feature 1564.

The contactless wound treatment barrier assembly 1500, 1600 employ a wound treatment barrier body support double sided adhesive 1540, 1640 for adhering the contactless wound treatment barrier assembly 1500, 1600 to the exemplary skin 510 of the patient. Wounds 512 have a tendency to drain or weep. The draining fluid may build up against the material of the contactless wound treatment barrier assembly 100, 300, 1500, 1600. The collected draining fluid commonly loosens and seeps through the adhesive interface. The uncontained seeping fluid can contaminate the region surrounding the wound; soil bedding, medical gowns, expose others to potential infections, and the like. It is desirous to contain any fluids seeping from the exemplary wound 512.

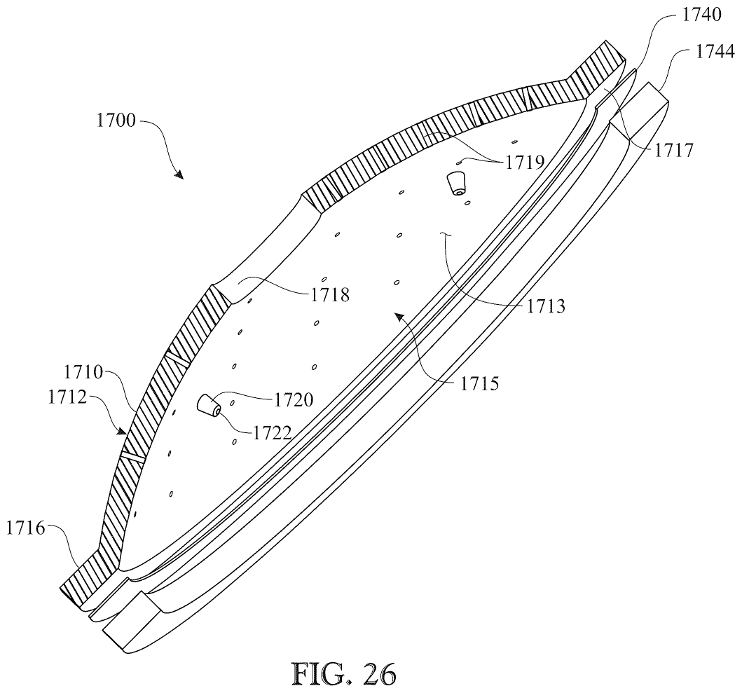

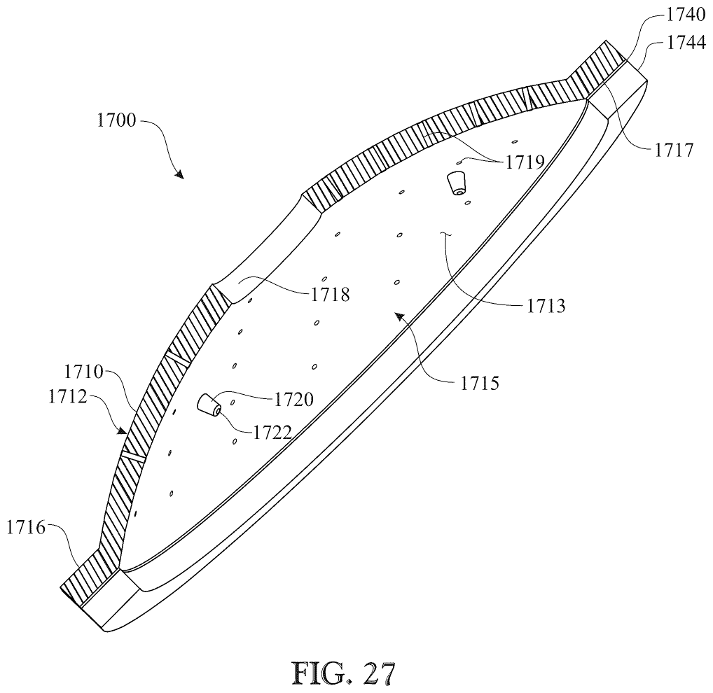

A contactless wound treatment barrier assembly 1700, illustrated in FIGS. 26 and 27, provides a solution that retains any seeping fluids within an interior of a wound treatment barrier body 1710. A majority of the elements of the contactless wound treatment barrier assembly 1700 and the contactless wound treatment barrier assembly 1500, 1600 are alike. Like elements of the contactless wound treatment barrier assembly 1700 and the contactless wound treatment barrier assembly 1500, 1600 are numbered the same, whereby the elements of the contactless wound treatment barrier assembly 1700 are preceded by the numeral “17”. A wound treatment barrier absorbent member 1744 is adhesively bonded to the wound treatment barrier body support flange contact surface 1715 of the wound treatment barrier body support flange 1716 of the wound treatment barrier body 1710 by a wound treatment barrier body support double sided adhesive 1740. A double sided adhesive (not illustrated) may or may not be applied to the exposed/contacting side of the wound treatment barrier absorbent member 1744. The wound treatment barrier absorbent member 1744 would be fabricated of a highly absorbent material. The wound treatment barrier absorbent member 1744 would be designed to have a volume that is capable of absorbing a significant volume of draining fluid to ensure against any draining fluids from escaping from the interior of the wound treatment barrier body 1710.

In an arrangement where the contactless wound treatment barrier assembly 1700 excludes a double sided adhesive (not illustrated) applied to the exposed/contacting side of the wound treatment barrier absorbent member 1744, an adhesive material can be applied to the exemplary wound 512 of the patient while trapping a portion of an outer surface of the wound treatment barrier body support flange 1716.

In an arrangement where the contactless wound treatment barrier assembly 1700 includes the double sided adhesive (not illustrated) applied to the exposed/contacting side of the wound treatment barrier absorbent member 1744, the exposed surface of the double sided adhesive material is adhesively secured to the exemplary wound 512 of the patient.

The contactless wound treatment barrier assembly 1500, 1600, 1700 is provided having a dome shape. The contactless wound treatment barrier assembly 1500, 1600, 1700 is designed to ensure a spatial distance is maintained between the exemplary wound 512 and the medication within the wound treatment composition carrier 1569, 1669, 1769 of the wound treatment composition storage and delivery container assembly 1550, 1650, 1750, thus ensuring against contact between the exemplary wound 512 and the medication within the wound treatment composition carrier 1569, 1669, 1769.

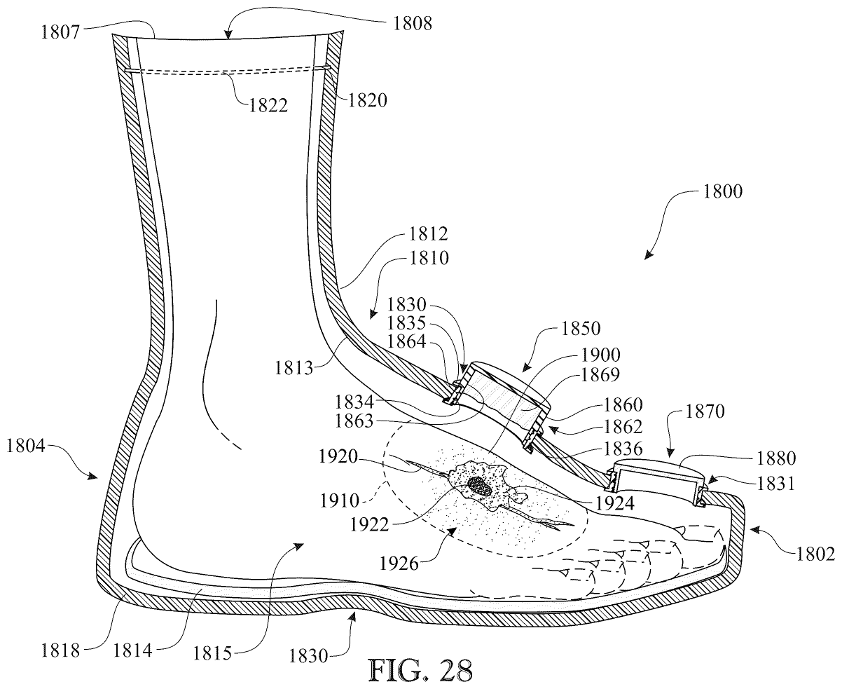

The arrangement of the dome shaped wound treatment barrier assembly 1500, 1600, 1700 is suitable for certain areas of the patient's body. A different solution is described herein for appendages, such as hands and feet. An exemplary foot covering contactless wound treatment barrier assembly 1800 is illustrated in FIG. 28. The exemplary foot covering contactless wound treatment barrier assembly 1800 is designed to be placed over a wounded foot 1900 of the patient. Although the exemplary foot covering contactless wound treatment barrier assembly 1800 is formed to be placed over the wounded foot 1900, it can be understood by those skilled in the art that the same arrangement can be modified for placement over a wounded hand of the patient.

The exemplary foot covering contactless wound treatment barrier assembly 1800 is formed having a J-shaped enclosure. A foot covering upper passageway edge 1807 provides a foot covering foot insertion/removal passageway 1808 into an interior of the foot covering contactless wound treatment barrier assembly 1800. The J-shaped enclosure is preferably fabricated of a pliant gas retaining material, such as Ethylene-Vinyl Acetate Copolymer (EVA foam) or Poly Ethylene-Vinyl Acetate Copolymer (PEVA). The EVA foam is a non-irritant to the patient and is iodine non-absorbent. Alternatively, the J-shaped enclosure can be fabricated of foam, rubber, silicone, silicone-rubber, nylon, flexible plastic, clear flexible plastic, leather, and vinyl, and the like.

The exemplary foot covering contactless wound treatment barrier assembly 1800 includes a foot covering wound treatment barrier body upper 1810 extending upward from a foot covering wound treatment barrier body lower 1818. The foot covering wound treatment barrier body upper 1810 and the foot covering wound treatment barrier body lower 1818 can be fabricated as a unitary assembly. Alternatively, the foot covering wound treatment barrier body upper 1810 can be assembled to the foot covering wound treatment barrier body lower 1818. In a condition where the patient desires to wear the foot covering contactless wound treatment barrier assembly 1800 during daily activities, such as walking, the foot covering wound treatment barrier body lower 1818 can be fabricated of a suitable material, such as a foam, rubber, nylon, flexible plastic, and the like, wherein the selected material would be suitable for use as a sole of a shoe. Orientation of the foot covering contactless wound treatment barrier assembly 1800 can be referenced by a foot covering toe section 1802 and a foot covering heel section 1804. Additionally, surfaces of the foot covering contactless wound treatment barrier assembly 1800 are identified by a foot covering wound treatment barrier body exterior surface 1812 and a foot covering wound treatment barrier body interior surface 1813.

A foot covering elastic sealing flange (diaphragm seal) 1820 is preferably assembled to an upper region of the foot covering wound treatment barrier body upper 1810. A foot covering elastic sealing flange limb pass through aperture 1822 is formed through the foot covering elastic sealing flange (diaphragm seal) 1820. The foot covering elastic sealing flange (diaphragm seal) 1820 is fabricated of an elastic material, such as a silicone material, a medical-grade silicone material, a silicone-rubber material, a rubber material, or any other suitable material having elastic properties. It is also preferred that the foot covering elastic sealing flange (diaphragm seal) 1820 be fabricated of a latex free material to reduce a risk of allergic reactions from patients. The foot covering elastic sealing flange limb pass through aperture 1822 is preferably of a size to provide a seal between the peripheral edge of the foot covering elastic sealing flange limb pass through aperture 1822 and the contacted portion of the patient, such as a lower portion of a leg of the patient, as illustrated in FIG. 28. The elastic properties of the foot covering elastic sealing flange (diaphragm seal) 1820 enable passage of the wounded foot 1900 of the patient therethrough.

Although the exemplary foot covering contactless wound treatment barrier assembly 1800 includes an opening 1808 spanning across an upper edge 1807 of the foot covering wound treatment barrier body upper 1810, similar to a boot, it is understood that the opening can be any suitable location and formation, such as along a partial length of the foot covering wound treatment barrier body upper 1810, along a portion of the heel region of the 1810 #, or any other suitable location.

Perforations, similar to those described above, can be provided through at least one of the material of the foot covering wound treatment barrier body upper 1810 and the foot covering elastic sealing flange (diaphragm seal) 1820.

At least one wound treatment composition container body receiving member 1830 can be assembled to the foot covering contactless wound treatment barrier assembly 1800 in any suitable location, such as through the foot covering wound treatment barrier body upper 1810 thereof, as illustrated. The wound treatment composition container body receiving member 1830 can be assembled to the foot covering contactless wound treatment barrier assembly 1800 using any suitable assembly method, such as ultrasonically welding two elements to one another sandwiching the material of the foot covering contactless wound treatment barrier assembly 1800 therebetween, using an adhesive, stitching the wound treatment composition container body receiving member 1830 to the material of the foot covering contactless wound treatment barrier assembly 1800, or any other suitable assembly method. The wound treatment composition container body receiving member 1830 can be used for connection to any optional item. A primary function of the wound treatment composition container body receiving member 1830 is for insertion, assembly, and retention of a wound treatment composition storage and delivery container assembly 1850, as illustrated. The wound treatment composition storage and delivery container assembly 1850 and the wound treatment composition storage and delivery container assembly 1550 comprise like elements. Like elements of the wound treatment composition storage and delivery container assembly 1850 and the wound treatment composition storage and delivery container assembly 1550 are numbered the same, wherein elements associated with the wound treatment composition storage and delivery container assembly 1850 and ancillary elements are preceded by the numeral “18”.

The foot covering contactless wound treatment barrier assembly 1800 would remain sealed prior to use. A cap (not illustrated but well understood by description) would be removed from the open portion of the wound treatment composition storage and delivery container body 1860. Removal of the cap exposes medication embedded within the wound treatment composition carrier 1869 located within an interior of the wound treatment composition storage and delivery container assembly 1850.

It is preferred that a shape of the foot covering wound treatment barrier body interior surface 1813 retains a spatial distance between the medication carried by the wound treatment composition carrier 1869 and a wound region 1910 on the encapsulated wounded foot 1900 of the patient. Alternatively, one or more elements can be introduced within the interior of the foot covering contactless wound treatment barrier assembly 1800 to maintain a spatial distance between the medication carried by the wound treatment composition carrier 1869 and the wound region 1910 on the encapsulated wounded foot 1900 of the patient. In yet another embodiment, the foot covering contactless wound treatment barrier assembly 1800 includes a second aperture, such as a removable viewing port 1880. A gas can be delivered into the interior of the foot covering contactless wound treatment barrier assembly 1800, wherein the gas maintains a separation between the foot covering wound treatment barrier body interior surface 1813 and the wounded foot 1900. More specifically, the gas maintains a separation between the wound treatment composition carrier 1869 and the wound region 1910.

In use, the cap or other seal would be removed from the wound treatment composition storage and delivery container assembly 1850. The wound treatment composition storage and delivery container body securing feature (threading in the exemplary illustration) 1864 would be engaged with the wound treatment composition container body receiving member threading 1834 of the wound treatment composition container body receiving member 1830. The inclusion of the interchangeable interface allows for replacement of spent wound treatment composition carrier 1869 without removal of the foot covering contactless wound treatment barrier assembly 1800 from the patient. The wound treatment composition carrier 1869 functions as previously described, wherein the wound treatment composition (iodine) that is embedded within the wound treatment composition carrier 1869 changes from a liquid or a solid state into a gaseous state. The gaseous state of the wound treatment composition disperses throughout the interior of the foot covering contactless wound treatment barrier assembly 1800, wherein the gaseous state of the wound treatment composition treats the wound 1910 while avoiding issues that are commonly encountered with direct contact of the same wound treatment composition.

It is preferred that the patient wear the foot covering contactless wound treatment barrier assembly 1800 over an extended period of time. Inclusion of a removable viewing port assembly 1870 enables visual inspection of the wound region 1910 to monitor a healing process of the deeper portion of the wound area 1922, the shallower portion of wound surrounding the deeper wound 1924, and the wound laceration 1920 of the wound region 1910. The removable viewing port assembly 1870 also enables visual inspection of the Iodine stain on the stratum corneum surrounding the wound area 1926 to monitor the level of iodine accumulation upon the patient.

The removable viewing port assembly 1870 is an assembly comprising a removable viewing port 1880 which is removably assembled to a receiving member annular ring 1831. The receiving member annular ring 1831 preferably replicates the wound treatment composition container body receiving member 1830. It would be desirous for the wound treatment composition storage and delivery container assembly 1850 and the removable viewing port 1880 to fit interchangeably within the wound treatment composition container body receiving member 1830 and the receiving member annular ring 1831. This also allows the wound treatment composition storage and delivery container assembly 1850 and the removable viewing port 1880 to be inserted into either location providing more direct wound care, improved monitoring of the wound region 1910, among other benefits.

A contactless wound treatment shoe insole 1814 can be inserted into the foot covering contactless wound treatment barrier assembly 1800 and placed upon an interior surface of the foot covering wound treatment barrier body lower 1818. The contactless wound treatment shoe insole 1814 is representative of any of the disclosed versions of the contactless wound treatment shoe insole 614, 714, 814, 1114, 1214.

Although the foot covering contactless wound treatment barrier assembly 1800 is illustrated to be worn on the wounded foot 1900 of the patient, the concept can be adapted to be worn on a hand of a patient. The shape of the adapted version of the foot covering contactless wound treatment barrier assembly 1800 would resemble a glove. The foot covering elastic sealing flange (diaphragm seal) 1820 would be adapted to have an arm covering elastic sealing flange (diaphragm seal) limb pass through aperture 1822 sized to provide a gaseous seal over an arm of the patient.

Although a portion of the exemplary wound treatment carriers are provided in a form of an insole, it can be understood that the same technology can be applied to similar materials and applied in a form of a bandage on other parts of a patient's body.

The above-described embodiments are merely exemplary illustrations of implementations set forth for a clear understanding of the principles of the invention. Many variations, combinations, modifications or equivalents may be substituted for elements thereof without departing from the scope of the invention. Therefore, it is intended that the invention not be limited to the particular embodiments disclosed as the best mode contemplated for carrying out this invention, but that the invention will include all the embodiments falling within the scope of the appended claims.

### ELEMENT DESCRIPTION REFERENCES

### Ref. No. Description

100 contactless wound treatment barrier assembly
110 wound treatment barrier body
112 wound treatment barrier body exterior surface
113 wound treatment barrier body interior surface
114 wound treatment barrier body peripheral contact edge
115 wound treatment barrier body interior volume
116 wound treatment barrier body support flange
117 wound treatment barrier body support flange contact surface
120 support flange adhesive
122 support flange adhesive protective cover
150 primed wound treatment composition storage and delivery container assembly (storage and container assembly)
160 primed wound treatment composition storage and delivery container body (storage and container body)
162 primed wound treatment composition storage and delivery container body exterior surface (storage and container body exterior surface)
163 primed wound treatment composition storage and delivery container body interior surface (storage and container body interior surface)
164 primed wound treatment composition storage and delivery container body attachment surface (storage and container body end wall or attachment surface)
165 primed wound treatment composition storage and delivery container closure (storage and container closure or storage and container cap)
166 wound treatment composition container
167 wound treatment composition
168 wound treatment composition carrier
169 primed wound treatment composition carrier
170 dense hook segment
172 dense loop segment
200 contactless wound treatment barrier assembly
210 wound treatment barrier body (barrier body)
212 wound treatment barrier body exterior surface (barrier body exterior surface)
213 wound treatment barrier body interior surface (barrier body interior surface)
214 wound treatment barrier body support flange (barrier body support flange or barrier body attachment flange)
215 wound treatment barrier body interior volume
217 wound treatment barrier body support flange contact surface (barrier body support flange contact surface or barrier body attachment flange contact surface)
230 contactless wound treatment barrier elastic retention member (elastic retention member)
232 elastic retention member fastener
300 contactless wound treatment barrier assembly
310 wound treatment barrier body
312 wound treatment barrier body exterior surface
313 wound treatment barrier body interior surface
314 wound treatment barrier body support flange
315 wound treatment barrier body interior volume
317 wound treatment barrier body support flange contact surface
350 primed wound treatment composition storage and delivery container assembly
360 primed wound treatment composition storage and delivery container body
362 primed wound treatment composition storage and delivery container body exterior surface
363 primed wound treatment composition storage and delivery container body interior surface
364 primed wound treatment composition storage and delivery container body attachment surface
365 primed wound treatment composition storage and delivery container closure
369 primed wound treatment composition carrier
370 dense hook segment
372 dense loop segment
380 vacuum creating element
382 vacuum retaining check valve
384 vacuum retaining check valve tubular member
386 vacuum retaining check valve flow control member
388 vacuum retaining check valve flow control member seat
400 contactless wound treatment barrier assembly
410 wound treatment barrier body
412 wound treatment barrier body exterior surface
413 wound treatment barrier body interior surface
414 wound treatment barrier body support flange
415 wound treatment barrier body interior volume
417 wound treatment barrier body support flange contact surface
416 support flange adhesive
418 support flange adhesive protective cover
450 primed wound treatment composition storage and delivery container assembly
460 primed wound treatment composition storage and delivery container body
462 primed wound treatment composition storage and delivery container body exterior surface
463 primed wound treatment composition storage and delivery container body interior surface
464 primed wound treatment composition storage and delivery container body attachment surface
465 primed wound treatment composition storage and delivery container closure
469 primed wound treatment composition carrier
470 dense hook segment
472 dense loop segment
482 interior pressure venting check valve
484 interior pressure venting check valve tubular member
486 interior pressure venting check valve flow control member
488 interior pressure venting check valve flow control member seat
490 air pump
491 air pump delivery tube
492 air fill check valve
494 air fill check valve tubular member
496 air fill check valve flow control member
498 air fill check valve flow control member seat
500 exemplary injured region
510 exemplary skin
512 exemplary wound
600 contactless wound treatment shoe assembly
602 contactless wound treatment shoe upper
604 contactless wound treatment shoe toe section
606 contactless wound treatment shoe heel section
610 contactless wound treatment shoe sole
612 contactless wound treatment shoe outsole
613 contactless wound treatment shoe insole seat
614 contactless wound treatment shoe insole
620 contactless wound treatment shoe insole base
622 contactless wound treatment shoe insole base body
623 insole base body upper surface
624 insole base body lower surface
628 contactless wound treatment shoe insole base first receptacle
629 contactless wound treatment shoe insole base second receptacle
630 contactless wound treatment shoe insole upper
632 contactless wound treatment shoe insole upper body
633 insole base upper body upper surface
634 insole base upper body lower surface
638 contactless wound treatment shoe insole upper first treatment delivery aperture
639 contactless wound treatment shoe insole upper second treatment delivery aperture
640 air flow generator
668 first wound treatment composition insert
669 second wound treatment composition insert
714 contactless wound treatment shoe insole
720 contactless wound treatment shoe insole lower
722 contactless wound treatment shoe insole lower body
723 insole lower body top surface
724 insole lower body bottom surface
726 insole lower body peripheral edge dam
730 contactless wound treatment shoe insole core
732 contactless wound treatment shoe insole core body
733 insole core body top surface
734 insole core body bottom surface
740 contactless wound treatment shoe insole upper
742 contactless wound treatment shoe insole upper body
743 insole upper body top surface
744 insole upper body bottom surface
814 wound treatment shoe insole
830 wound treatment shoe insole core
832 wound treatment shoe insole core body
833 insole core body top surface
834 insole core body bottom surface
840 wound treatment shoe insole upper
842 wound treatment shoe insole upper body
843 insole upper body top surface
844 insole upper body bottom surface
867 wound treatment composition
900 contactless wound treatment flow diagram
910 apply wound treatment composition to carrier step
912 seal wound treatment composition carrier step
914 unseal wound treatment composition carrier step
920 secure wound composition carrier to interior of support element step
922 prepare support element for placement and attachment to the body at a location covering the wound step
924 place support element on the body at the location covering the wound step
926 secure support element to the body at the location covering the wound step
930 treat wound by gaseous application of wound treatment composition around the wound step
940 remove support element from the body after a period of time step
950 repeat application process as needed step
1000 elemental iodine impregnated insole fabrication process flow diagram
1010 incorporate elemental iodine into polyurethane
1012 dissolve elemental iodine into Polyols
1020 treat composition with Di-isocyanates
1030 form polyurethane into insole
1114 wound treatment shoe insole
1130 wound treatment shoe insole core
1132 wound treatment shoe insole core body
1134 insole core body bottom surface
1140 wound treatment shoe insole microporous polyurethane foam
1142 wound treatment shoe insole microporous polyurethane foam body
1143 insole microporous polyurethane foam top surface
1214 wound treatment shoe insole
1220 contactless wound treatment shoe insole spacing member
1222 contactless wound treatment shoe insole spacing member base
1223 insole spacing member base top surface
1224 insole spacing member base bottom surface
1226 insole spacing member peripheral frame
1226D insole spacing member peripheral frame depth
1228 insole spacing member base interior volume
1230 wound treatment shoe insole core
1232 wound treatment shoe insole core body
1232T wound treatment shoe insole core body thickness
1233 insole core body top surface
1234 insole core body bottom surface
1260 wound treatment composition storage and delivery container body
1262 wound treatment composition delivery nozzle
1267 wound treatment composition
1300a wounded foot after one (1) week of treatment
1300b wounded foot after two (2) weeks of treatment
1300c healed foot after three (3) weeks of treatment
1310a wound after one (1) week of treatment
1310b wound after two (2) weeks of treatment
1310c healed wound after three (3) weeks of treatment
1320a laceration wound after one (1) week of treatment
1320b laceration nearly or completely healed after two (2) weeks of treatment
1320c laceration after three (3) weeks of treatment
1322a deeper portion of the wound area after one (1) week of treatment
1322b deeper portion of the wound after two (2) weeks of treatment
1322c deeper portion of the wound after three (3) weeks of treatment
1324a shallower portion of wound surrounding the deeper wound after one (1) week of treatment
1324b shallower portion of the wound after two (2) weeks of treatment
1326 Iodine stain on stratum corneum surrounding the wound area after one (1) week of treatment
1400 odor treatment process flow diagram
1410 place odor treatment composition carrier in footwear step
1412 insert carrier and footwear into bag/enclosure step
1414 seal bag/enclosure step
1416 pass time allowing outgassing of odor treatment composition step
1418 embed outgassed odor treatment composition into footwear step
1420 remove footwear from bag/enclosure step
1430 place footwear onto foot of patient step
1432 wear composition embedded footwear to treat odor step
1440 remove footwear from foot when done step
1500 contactless wound treatment barrier assembly
1510 wound treatment barrier body
1512 wound treatment barrier body exterior surface
1513 wound treatment barrier body interior surface
1515 wound treatment barrier body interior volume
1516 wound treatment barrier body support flange
1517 wound treatment barrier body support flange contact surface
1518 wound treatment barrier body wound composition container body receiving aperture
1519 wound treatment barrier body perforation
1520 diffuser/heater
1522 diffuser/heater nozzle
1530 wound treatment barrier body viewing port
1532 wound treatment barrier body viewing port clear seal
1540 wound treatment barrier body support double sided adhesive
1542 wound treatment barrier body support double sided adhesive protectent
1550 wound treatment composition storage and delivery container assembly
1560 wound treatment composition storage and delivery container body
1562 wound treatment composition storage and delivery container body exterior surface
1563 wound treatment composition storage and delivery container body interior surface
1564 wound treatment composition storage and delivery container body securing feature
1569 wound treatment composition carrier
1600 contactless wound treatment barrier assembly
1610 wound treatment barrier body
1612 wound treatment barrier body exterior surface
1613 wound treatment barrier body interior surface
1615 wound treatment barrier body interior volume
1616 wound treatment barrier body support flange
1617 wound treatment barrier body support flange contact surface
1618 wound treatment barrier body wound composition container body receiving aperture
1619 wound treatment barrier body perforation
1630 wound treatment composition container body receiving member
1634 wound treatment composition container body receiving member threading
1635 wound treatment composition container body receiving member exterior flange
1636 wound treatment composition container body receiving member annular ring
1637 wound treatment composition container body receiving member interior flange
1650 wound treatment composition storage and delivery container assembly
1660 wound treatment composition storage and delivery container body
1662 wound treatment composition storage and delivery container body exterior surface
1663 wound treatment composition storage and delivery container body interior surface
1664 wound treatment composition storage and delivery container body securing feature
1669 wound treatment composition carrier
1700 contactless wound treatment barrier assembly
1710 wound treatment barrier body
1712 wound treatment barrier body exterior surface
1713 wound treatment barrier body interior surface
1715 wound treatment barrier body interior volume
1716 wound treatment barrier body support flange
1717 wound treatment barrier body support flange contact surface
1718 wound treatment barrier body wound composition container body receiving aperture
1719 wound treatment barrier body perforation
1720 diffuser/heater
1722 diffuser/heater nozzle
1740 wound treatment barrier body support double sided adhesive
1744 wound treatment barrier absorbent member
1750 wound treatment composition storage and delivery container assembly
1760 wound treatment composition storage and delivery container body
1762 wound treatment composition storage and delivery container body exterior surface
1763 wound treatment composition storage and delivery container body interior surface
1764 wound treatment composition storage and delivery container body securing feature
1769 wound treatment composition carrier
1800 foot covering contactless wound treatment barrier assembly
1802 foot covering toe section
1804 foot covering heel section
1807 foot covering upper passageway edge
1808 foot covering foot insertion/removal passageway
1810 foot covering wound treatment barrier body upper
1811 foot covering contactless wound treatment barrier body interior volume
1812 foot covering wound treatment barrier body exterior surface
1813 foot covering wound treatment barrier body interior surface
1814 contactless wound treatment shoe insole
1818 foot covering wound treatment barrier body lower
1820 foot covering elastic sealing flange (diaphragm seal)
1822 foot covering elastic sealing flange (diaphragm seal) limb pass through aperture
1830 wound treatment composition container body receiving member
1831 receiving member annular ring
1834 wound treatment composition container body receiving member threading
1835 wound treatment composition container body receiving member exterior flange
1836 wound treatment composition container body receiving member annular ring
1850 wound treatment composition storage and delivery container assembly
1860 wound treatment composition storage and delivery container body
1862 wound treatment composition storage and delivery container body exterior surface
1863 wound treatment composition storage and delivery container body interior surface
1864 wound treatment composition storage and delivery container body securing feature
1869 wound treatment composition carrier
1870 removable viewing port assembly
1880 removable viewing port
1900 wounded foot
1910 wound region
1920 wound laceration
1922 deeper portion of the wound area
1924 shallower portion of wound surrounding the deeper wound
1926 Iodine stain on stratum corneum surrounding the wound area

TABLE 1
Evidence Based Complimentary and Alternative Medicine

| MAPs | Part used | Major chemical compounds | Inhibited microorganisms |
|---|---|---|---|
| Achillea clavennae | Leaves and flowers | Camphor, myrcene, 1,8-cineole, | Klebsiella pneumonia, |
| | | β-caryophyllene, linalool, geranyl acetate | Streptococcus pneumonia, |
| | | | Haemophilus influenzae, |
| | | | Pseudomonas aeruginosa |
| Achillea | Aerial parts | Yomogi alcohol, 1,8-cineole, artemisia alcohol, | Staphylococcus aureus, |
| fragrantissima | | thujone | Staphylococcus epidermidis, |
| | | | Escherichia coli |
| Achillea ligustica | Aerial parts | Viridiflorol, terpinen-4-ol | Streptococcus mutans |
| Artemisia | Aerial parts | Myrcene, trans-thujone, trans-sabinyl acetate | E. coli, S. aureus, |
| absinthium | | | Staphylococcus epidermidis |
| Artemisia biennis | Aerial parts | (Z)-Beta-ocimene, (E)-beta-farnesene, | E. coli, S. aureus, |
| | | acetylenes, (Z)- and (E)-En-yn-dicycloethers | S. epidermidis |
| Artemisia cana | Aerial parts | Santolina triene, alpha-pinene, camphene | E. coli, S. aureus, |
| | | | S. epidermidis |
| Artemisia dracunculus | Aerial parts | Methylchavicol, methyl eugenol, | E. coli, S. aureus, |
| | | beta-phellandrene, terpinolene | S. epidermidis, Brochothrix |
| | | | thermosphacta, Listeria |
| | | | innocua, L. monocytogenes, |
| | | | Pseudomonas putida, |
| | | | Shewanella putrefaciens |
| Artemisia longifolia | Aerial parts | Alpha-pinene, camphene, 1,8-cineole | E. coli, S. aureus, |
| | | | S. epidermidis |
| Artemisia frigida | Aerial parts | 1,8-Cineole, methylchavicol, camphor | E. coli, S. aureus, |
| | | | S. epidermidis |
| Cinnamomum | Bark, leaves | Cinnamaldehyde | Enterobacteriaceae, |
| zeylancium | | | S. aureus, Streptococcus |
| | | | pyogenes, S. pneumoniae, |
| | | | Enterococcus faecalis, |
| | | | E. faecium, Bacillus cereus, |
| | | | Acinetobacter lwoffii, |
| | | | Enterobacter aerogenes, |
| | | | E. coli, Klebsiella pneumoniae, |
| | | | Proteus mirabilis, |
| | | | P. aeruginosa, Salmonella |
| | | | typhimurium, Clostridium |
| | | | perfringens, |
| | | | Mycobacterium smegmatis |
| Copaifera officinalis | Essential oil | β-Caryophyllene, β-bisabolene, germacrene B, | S. aureus, E. coli |
| | | α-copaene, germacrene D, α-humulene, | |
| | | δ-cadinene | |
| Coriandrum sativum | Leaves | 2E-Decenal, decanal, 2E-decen-1-ol, n-decanol | S. aureus, Bacillus spp., |
| | | | E. coli, Salmonella typhi, |
| | | | K. pneumonia, Proteus |
| | | | mirabilis, P. aeruginosa |
| Cuminum cyminum | Leaves | γ-Terpin-7-al, γ-terpinene, β-pinene, | S. typhimurium, E. coli |
| | | cuminaldehyde | |
| Cymbopogon citratus | Fruit | Ethanolic compounds | Enterobacteriaceae, |
| | | | S. aureus |
| Cymbopogon nardus | Leaves, stems | Δ2-Carene, beta-citronellal | Brochothrix thermosphacta, |
| | | | E. coli, Listeria innocua, |
| | | | L. monocytogenes, P. putida, |
| | | | S. typhimurium, |
| | | | S. putrefaciens |
| Cyperus longus | Arial part | β-Himachalene, α-humulene, γ-himachalene | S. aureus, |
| | | | L. monocytogenes, |
| | | | L. monocytogenes, E. faecium, |
| | | | S. Enteritidis, E. coli, |
| | | | P. aeruginosa |
| Daucus littoralis | Leaves, stems, roots, | Germacrene D, acorenone B | S. aureus, E. coli |
| | flowers, fruits | | |
| Dracocephalum | Leaves | n-Mentha-1,8-dien-10-al, limonene, geranial, | B. subtilis, S. aureus, |
| foetidum | | neral | M. luteus, E. hirae, |
| | | | S. mutans, E. coli |
| Eremanthus | Leaves | (Z)-Caryophyllene, germacrene D, viridiflorol, | S. epidermidis |
| erythropapps | | p-cymene, γ-terpinene | |
| Eugenia | Flower buds | Phenylpropanoids such as carvacrol, thymol, | S. epidermidis |
| caryophyllata | | eugenol, cinnamaldehyde | |
| Euphrasia rostkoviana | Essential oil | n-Hexadecanoic acid, thymol, myristic acid, | E. faecalis, E. coli, |
| | | linalool | K. pneumoniae, S. aureus, |
| | | | S. epidermidis, P. aeruginosa |
| Foeniculum vulgare | Leaves | Trans-anethole, methylchavicol, limonene | S. typhimurium, E. coli |
| Fortunella margarita | Leaves | Gurjunene, eudesmol, muurolene | B. subtilis, S. aureus, |
| | | | Sarcina luta, S. faecalis, |
| | | | E. coli, K. pneumonia, |
| | | | P. aeruginosa |
| Juniperus phoenicea | Arial part | α-Pinene, β-phellandrene, α-terpinyl acetate | S. aureus, |
| | | | L. monocytogenes, |
| | | | L. monocytogenes, E. faecium, |
| | | | S. Enteritidis, E. coli, |
| | | | P. aeruginosa |
| Laurus nobilis | Arial part | Eucalyptol (1,8-cineole), linalool | Mycobacterium smegmatis, |
| | | | E. coli |
| Lavandula × intermedia | Arial part | Camphor, eucalyptol (1,8-cineole), linalool, | M. smegmatis, E. coli |
| ‘Provence’ (Blue | | β-pinene, α-pinene | |
| Lavandin) (a cross between | | | |
| L. angustifolia, | | | |
| L. Latifolia) | | | |
| Juniperus excelsa | Leaves and twigs | α-Pinene, α-cedrol, δ-car-3-ene | S. aureus |
| Lippia sidoides | Leaves | Thymol and carvacrol | S. mutans, S. sanguis, |
| | | | S. salivarius, S. mitis |
| Mentha piperita | Arial part | | S. aureus, S. typhimurium, |
| | | | V. parahaemolyticus |
| Mentha pulegium | Arial part | Piperitone, piperitenone, α-terpineol, pulegone | S. aureus, S. epidermidis, |
| | | | B. cereus, L. monocytogenes, |
| | | | E. coli, S. typhimurium, |
| | | | V. cholera, L. monocytogenes, |
| | | | E. faecium, S. Enteritidis |
| Mentha suaveolens | Arial part | Pulegone, piperitone, cis-cis-p-menthenolide, | Lactococcus lactis subsp. |
| | | limonene germacrene | Lactis, S. xylosus |
| Melaleuca alternifolia | Essential oil | Terpinen-4-ol, 1,8-cineole, γ-terpinene, | E. coli, S. aureus, |
| (tea tree oil) | | α-terpinene, terpinolene | S. epidermidis, E. faecalis, |
| | | | P. aeruginosa, M. avium, |
| | | | H. influenzae, S. pyogenes, |
| | | | S. pneumonia |
| Momordica charantia | Seed | Trans-nerolidol, apiole, cis-dihydrocarve, | E. coli, S. aureus |
| | | ol germacrene D | |
| Myrtus | Leaves | Eugenol, α-terpineol, γ-terpinene | S. aureus, |
| communis | | | L. monocytogenes, E. durans, |
| | | | Salmonella Typhi, E. coli, |
| | | | B. subtilis, M. tuberculosis, |
| | | | P. aeruginosa, K. pneumonia, |
| | | | M. avium subsp. paratuberculosis, |
| | | | E. cloacae |
| Nigella sativa | Seeds | Thymoquinone, p-cymene, α-thujene, | S. aureus, B. cereus, E. coli, |
| | | thymohydroquinone, longifolene | P. aeruginosa |
| Ocimum | Leaves | Eugenol, methyl eugenol, cis-ocimene, | S. aureus, Bacillus spp. |
| gratissimum | | trans-ocimene, α-pinene, camphor | E. coli, P. aeruginosa, S. typhi, |
| | | | K. pneumoniae, |
| | | | P. mirabilis, E. cloacae |
| Ocimum | Flowers and leaves | Eugenol, borneol, linalool, methyl eugenol | B. subtilis, S. aureus, |
| kilimandscharicum | | | Citrobacter youngae, E. coli, |
| | | | Klebsiella spp., Micrococcus |
| | | | spp., Proteus spp., |
| | | | Pseudomonas spp., |
| | | | Salmonella spp. |
| Origanum vulgare | Leaves, Arial part | Carvacrol, thymol, γ-terpinene, trans-sabinene | Clostridium botulinum, |
| | | hydrate, cis-piperitol, borneol, terpinen-4-ol, | C. perfringens, |
| | | linalool | L. monocytogenes, E. coli, |
| | | | S. choleraesuis, |
| | | | S. typhimurium, S. aureus, |
| | | | B. subtilis, Pseudomonas |
| | | | aeruginosa, Shigella sonnei, |
| | | | Sarcina lutea, M. flavus, |
| | | | K. pneumoniae, K. oxytoca |
| Ocimum basilicum | Leaves, stems | γ-Terpinene, methylchavicol | Brochothrix thermosphacta, |
| | | | E. coli, L. innocua, |
| | | | L. monocytogenes, P. putida, |
| | | | S. typhimurium, |
| | | | S. putrefaciens, M. flavus |
| Petroselinum | Leaves, stems | Myristicin, apiol, | B. thermosphacta, E. coli, |
| sativum | | 1,2,3,4-tetramethoxy-5-(2-propenyl)- | L. innocua, L. monocytogenes, |
| | | benzene | P. putida, S. typhimurium, |
| | | | S. putrefaciens |
| Piper nigrum | Essential oil | Limonene, δ-3-carene, α-pinene, | S. aureus, E. coli |
| | | β-caryophyllene, β-pinene, sabinene, | |
| | | α-felandeno, myrcene, para-cymene, linalool, | |
| | | terpinolene, β-selinene, 1,8 cineole, | |
| | | α-terpinene, α-humulene, α-copaene, eugenol, | |
| | | terpinen-4-ol, camphene, safrole | |
| Pimpinella anisum | Seed | Trans-anethole | S. typhimurium, E. coli |
| Plectranthus | Leaves | (Z)-Caryophyllene, germacrene D, viridiflorol, | S. epidermidis |
| barbatus | | p-cymene, γ-terpinene | |
| P. amboinicus | Leaves | (Z)-Caryophyllene, germacrene D, viridiflorol, | S. epidermidis |
| | | p-cymene, γ-terpinene | |
| Plectranthus | Leaves | α-Pinene, β-pinene, trans-caryophyllene, | E. faecalis, S. salivarius, |
| neochilus | | caryophyllene oxide | S. sobrinus, S. sanguinis, |
| | | | S. mitis, L. casei, S. mutans |
| Pogostemon cablin | Leaves | Patchoulol, δ-guaieno; gurjunene-α, α-guaiene, | K. pneumonia, H. pylori, |
| | | aromadendrene, β-patchoulene | E. coli, B. subtilis, S. aureus, |
| | | | P. aeruginosa, E. faecalis |
| Rosmarinus | Leaves, flower | Camphor, camphene, limonene, geraniol, | E. coli, S. typhimurium, |
| officinalis | | myrcene, linalool benzoylacetate, linalool, | B. cereus, Bacillus subtilis, |
| | | α-pinene, α-terpinolene, bornyl acetate, | S. aureus, S. agalactiae, |
| | | borneol | S. epidermidis, S. aureus, |
| | | | P. vulgaris, P. aeruginosa, |
| | | | K. pneumonia, E. faecalis, |
| | | | B. thermosphacta, L. innocua, |
| | | | L. monocytogenes, |
| | | | P. putida, S. typhimurium, |
| | | | S. putrefaciens, M. smegmatis |
| Satureja hortensis | Arial part | Carvacrol, thymol, γ-terpinene | C. botulinum, |
| | | | C. perfringens, |
| Salvia sclarea | Arial part | Linalool, linalyl acetate, geranyl acetate, β- | S. aureus, S. agalactiae, |
| | | ocimene acetate, caryophyllene oxide | S. epidermis, E. coli, Proteus |
| | | | vulgaris, P. aeruginosa, |
| | | | K. pneumonia, E. faecalis, |
| | | | B. pumilus, B. subtilis, |
| | | | S. typhimurium |
| Salvia officinalis | Arial part | α-Thujone, camphor, 1,8-cineole, α-pinene | S. aureus, P. stuartii, |
| | | | P. stuartii, E. coli, Shigella |
| | | | sonnei, Sarcina lutea, |
| | | | M. flavus, B. thermosphacta, |
| | | | E. coli, L. innocua, |
| | | | L. monocytogenes |
| Salvia lavandulifolia | Essential oil | Camphor, α-thujone, beta-thujone, camphene, | P. vulgaris, P. aeruginosa, |
| | | α-pinene, terpineol | K. pneumonia, E. faecalis |
| Satureja cuneifolia | Aerial parts | Carvacrol and p-cymene | E. coli, Campylobacter |
| | | | jejuni, S. sonnei, S. aureus, |
| | | | L. monocytogenes, |
| | | | B. cereus, P. aeruginosa, |
| | | | S. enteritidis |
| Struchium | Leaves | β-Caryophyllene, germacrene A, α-humulene, | S. typhi, B. cereus, |
| sparganophora | | germacrene D | P. mirabilis, P. aeruginosa, |
| | | | B. subtilis |
| Syzygium aromaticum | Leaves, flower bud | Eugenol, eugenylacetate | P. aeruginosa, |
| | | | Enterobacteriaceae |
| Syzygium cumini | Leaves | α-Pinene, β-pinene, trans- caryophyllene, | E. coli, S. aureus, |
| | | 1,3,6-octatriene, delta-3-carene, | P. aeruginosa, |
| | | α-caryophyllene, α-limonene | N. gonorrhoeae, B. subtilis, |
| | | | S. aureus |
| Trachyspermum | Seeds | — | K. pneumoniae, E. coli, |
| ammi | | | S. aureus |
| Thymus vulgaris | Arial part | Thymol, linalool, carvacrol, 1,8-cineole, | L. monocytogenes, E. coli, |
| | | eugenol, camphor, camphene, α-pinene, | S. typhimurium, S. aureus, |
| | | borneol, β-pinene | C. botulinum, C. perfringens, |
| | | | S. sonnei, S. lutea, |
| | | | M. flavus, B. thermosphacta, |
| | | | L. innocua, L. monocytogenes, |
| | | | P. putida, S. putrefaciens |
| Thymus zygis | Essential oil | — | S. choleraesuis, |
| | | | S. typhimurium, E. coli |
| Thymus mastichina | Leaves, stems | m-Thymol, carvacrol, trans-caryophyllene | B. thermosphacta, E. coli, |
| | | | L. innocua, L. monocytogenes, |
| | | | P. putida, S. typhimurium, |
| | | | S. putrefaciens |
| Thymus kotschyanus | Arial part | Carvacrol, 1,8 cineole, thymol, borneol, | S. aureus, S. epidermidis, |
| | | E-caryophyllene | B. cereus, E. coli |
| Thuja sp. | Essential oil | Alpha-thujone and beta-thujone | P. aeruginosa, |
| (Thuja plicata, | | | K. pneumoniae, S. aureus, |
| Thuja occidentalis) | | | E. coli |
| Verbena officinalis | Arial part | Borneol, geranoil | S. aureus, E. coli, |
| | | | S. typhimurium, |
| | | | L. monocytogenes |
| Warionia saharae | Arial part | β-Eudesmol, trans-nerolidol, linalool, 1,8 | S. aureus, B. cereus, |
| | | cineole, camphor, p-cymene, terpinen-4-ol | P. aeruginosa, E. coli |

TABLE 2
Exemplary Plant Based Wound Treatment Compositions

| MAPs | Part used | Major chemical compounds | Inhibited microorganisms |
|---|---|---|---|
| Aegle marmelos | Leaves | γ-Cadinene, δ-carene, | Candida albicans, Aspergillus niger, |
| | | α-pinene | Fusarium oxysporum |
| Artemisia biennis | Aerial parts | (Z)-β-Ocimene, | Cryptococcus neoformans, Fonsecaea pedrosoi, |
| | | (E)-beta-farnesene, | A. niger |
| | | acetylenes, (Z)- and | |
| | | (E)-en-yn-dicycloethers | |
| Cinnamomum | Bark, leaves | Cinnamaldehyde | C. albicans, C. parapsilosis, C. krusei |
| zeylancium | | | |
| Coriandrum sativum | Leaves | 2E-Decenal, decanal, | C. albicans |
| | | 2E-decen-1-ol, n-decanol | |
| Daucus littoralis | Leaves, stems, | Germacrene D, acorenone B | C. albicans |
| | roots, flowers, | | |
| | fruits | | |
| Dracocephalum | Leaves | n-Mentha-1,8-dien-10-al, | C. albicans |
| foetidum | | limonene, geranial, neral | |
| Eremanthus | Leaves | (Z)-Caryophyllene, | C. albicans, C. gattii, C. gattii, C. neoformans, |
| erythropappus | | germacrene D, viridiflorol, | S. cerevisiae |
| | | p-cymene, γ-terpinene | |
| Euphrasia rostkoviana | Essential oil | n-Hexadecanoic acid, thymol, | C. albicans |
| | | myristic acid, linalool | |
| Feoniculum vulgare | Seed | Trans-anethole, | Alternaria alternata, F. oxysporum, A. flavus |
| | | methylchavicol, limonene | |
| Fortunella margarita | Leaves | Gurjunene, eudesmol, | A. niger, C. albicans |
| | | muurolene | |
| Glechon spathulata | Leaves | β-Caryophyllene, | Trichophyton rubrum, Epidermophyton floccosum |
| | | bicyclogermacrene | |
| Glechon marifolia | Leaves | β-Caryophyllene, | T. rubrum, E. floccosum |
| | | bicyclogermacrene | |
| Lippia sidoides | Leaves | Thymol and carvacrol | C. albicans |
| Melaleuca alternifolia | Essential oil | Terpinen-4-ol, 1,8-cineole, | Alternaria spp. A. flavus, A. fumigates, A. niger, |
| (tea tree oil) | | γ-terpinene, α-terpinene, | Blastoschizomyces Capitatus, C. albicans, |
| | | terpinolene | C. glabrata, C. parapsilosis, C. tropicalis, |
| | | | Cladosporium spp., C. neoformans, |
| | | | Epidermophyton floccosum, Fusarium spp., |
| | | | Malassezia furfur, Microsporum canis, |
| | | | M. sympodialis, M. gypseum, Penicillium spp., |
| | | | Rhodotorula rubra, Saccharomyces cerevisiae, |
| | | | Trichophyton mentagrophytes, T. rubrum, |
| | | | T. tonsurans, Trichosporon spp. |
| Mentha pulegium | Arial part | Piperitone, piperitenone, | A. niger, C. albicans, C. zemplinina, |
| | | α-terpineol pulegone | Kloeckera apiculata, Metschnikowia pulcherrima, |
| | | | Tetrapisispora phaffii |
| Momordica charantia | Seed | Trans-nerolidol, apiole, | C. albicans |
| | | cis-dihydrocarveol, | |
| | | germacrene D | |
| Myrtus communis | Leaves | Eugenol, α-terpineol, | C. albicans, A. flavus |
| | | γ-terpinene, α-caryophyllene | |
| Nigella sativa | Seeds | Thymoquinone, p-cymene, | A. flavus, Fusarium moniliforme, F. graminearum, |
| | | α-thujene, | P. viridicatum |
| | | thymohydroquinone, | |
| | | longifolene | |
| Ocimum species | Leaves, flower | Eugenol, methyl eugenol, | C. albicans, C. tropicalis, C. glabrata, P. notatum, |
| (Ocimum basilicum, | | cis-ocimene, trans-ocimene, | R. stolonifer, M. mucedo, A. ochraceus, A. versicolor, |
| Ocimum gratissimum, | | α-pinene camphor | A. niger, A. fumigates, T. viride, P. funiculosum |
| O. kilimandscharicum, | | | |
| O. lamiifolium, | | | |
| O. suave) | | | |
| Origanum vulgare | Leaves, arial part | Carvacrol, thymol, | C. albicans, A. niger, M. gypseum, M. canis, |
| | | γ-terpinene, trans-sabinene | A. cajetani, T. violaceum, T. mentagrophytes, |
| | | hydrate, cis-piperitol, borneol, | E. floccosum, T. rubrum, T. tonsurans, |
| | | terpinen-4-ol, linalool | phytopathogens B. cinerea and P. oryzae |
| Pelargonium | Leaves | Citronellol, citronellyl | C. tropicalis |
| graveolens | | formate, geraniol | |
| Plectranthus barbatus | Leaves | (Z)-Caryophyllene, | C. albicans, C. gattii, C. gattii, C. neoformans, |
| and P. amboinicus | | germacrene D, viridiflorol, | S. cerevisiae. |
| | | p-cymene, γ-terpinene | |
| Pogostemon cablin | Leaves | Patchoulol, δ-guaieno; | Aspergillus species, C. albicans |
| | | gurjunene-α, α-guaiene, | |
| | | aromadendrene, β-patchoulene | |
| Rosmarinus officinalis | Leaves | Camphor, camphene, | C. albicans, M. gypseum, M. canis, A. cajetani, |
| | | limonene, geraniol, myrcene, | T. violaceum, T. mentagrophytes, E. floccosum, T. rubrum, |
| | | linalool benzaylacetate, | T. tonsurans, phytopathogens B. cinerea, |
| | | linalool, α-pinene, | P. oryzae |
| | | α-terpinolene, bornyl acetate, | |
| | | borneol | |
| Salvia sclarea | Arial part | Linalool, linalyl acetate, | C. albicans, C. tropicalis, C. krusei, C. glabrata, |
| | | geranyl acetate, β- ocimene | C. parapsilosis |
| | | acetate, caryophyllene oxide | |
| Syzygium aromaticum | Leaves | Eugenol, eugenylacetate | A. fumigatus, C. albicans, Candida spp. |

TABLE 3
Exemplary Essential Oils and Respective Antiviral Activity Against Human Pathogens

| Plant | Part used | Chemical compounds | Inhibited microorganisms |
|---|---|---|---|
| Achillea | Aerial parts | 2,5,5-Trimethyl-3,6- | ORF virus |
| fragrantissima | | heptadien-2-ol, eucalyptol, | (a parapox virus) |
| | | artemisia alcohol, thujone | |
| Artemisia | Aerial parts | β-Thujone, linalool, | Herpes simplex virus type 1 |
| arborescens | | myrcene, carvacrol | (HSV-1) |
| Fortunella | Leaves | Gurjunene, eudesmol, | Avian influenza A virus |
| margarita | | muurolene | (H5N1), |
| Glechon spathulata | Leaves | β-Caryophyllene, | HSV-1 |
| | | bicyclogermacrene | |
| Glechon marifolia | Leaves | β-Caryophyllene, | HSV-1 |
| | | bicyclogermacrene | |
| Hyptis mutabilis | Leaves | α-Phellandrene, p-cymene, | HSV-1 |
| | | E-caryophyllene | |
| Lepechinia | Leaves | Germacrene D | HSV-1 |
| salviifolia | | | |
| Melissa officinalis | Leaves | Myrcene, linalool, | HSV-2, avian influenza |
| | | camphor, citronellal, | virus (AIV) subtype H9N2 |
| | | β-caryophyllene, | |
| | | caryophyllene oxide, citral | |
| Minthostachys | Leaves | α-Pinene, estragole | HSV-1 |
| mollis | | | |
| Ocimum campechianum | Leaves | Linalool, eugenol | HSV-1 |
| Pogostemon cablin | Leaves | Patchoulol, δ-guaieno; | Influenza A (H2N2) virus |
| | | gurjunene-α, α-guaiene, | |
| | | aromadendrene, | |
| | | β-patchoulene | |
| Trachyspermum | Leaves | Thymol, α-pinene, | Japanese encephalitis virus |
| ammi | | p-cymene, limonene | (JEV) |

What is claimed is:

1. A contactless medicine applicating apparatus comprising:
a wound treatment barrier comprising a volume encapsulating body, wherein an interior surface of the volume encapsulating body defines a gas retaining volume;
a wound treatment composition carrier, the wound treatment composition carrier carrying a wound treatment composition in at least one of a solid state and a liquid state, wherein the wound treatment composition carrier has a property enabling outgassing of the wound treatment composition in a gaseous state;
a wound treatment composition carrier receiving aperture formed through the volume encapsulating body, the wound treatment aperture is of a shape and size to receive and retain the wound treatment composition carrier,
wherein the wound treatment composition contains iodine,
wherein in use, the wound treatment composition carrier is removably inserted into the wound treatment composition carrier receiving aperture and a gaseous state of the wound treatment composition is discharged into the gas retaining volume defined by the interior surface of the volume encapsulating body,
wherein the volume encapsulating body is of a shape and size to encapsulate an exposed wound of a patient and at least one sealing element that provides a substantially gas impervious seal against skin of a patient;
wherein the volume encapsulating body is of a shape and size to encapsulate the exposed wound of the patient while avoiding contact between the wound treatment composition carrier and the exposed wound of the patient.

2. The contactless medicine applicating apparatus as recited in claim 1, wherein the volume encapsulating body is provided in one of the following shapes:
(a) a dome shape,
(b) a dome shape having a flange extending radially outward from a peripheral edge thereof,
(c) a J-shape,
(d) a J-shape having a seal located proximate an opening at a top of the J-shape,
(e) a foot covering,
(f) a foot covering having a seal located proximate an opening at an insertion region of the foot covering, and
(g) a glove.

3. The contactless medicine applicating apparatus as recited in claim 1, the wound treatment composition carrier further comprising a mechanically assembling feature for assembling the wound treatment composition carrier within the wound treatment aperture of the volume encapsulating body.

4. The contactless medicine applicating apparatus as recited in claim 1, the apparatus further comprising a wound treatment composition container body receiving member inserted through the wound treatment composition carrier receiving aperture.

5. The contactless medicine applicating apparatus as recited in claim 4, the wound treatment composition container body receiving member further comprising threading for threadably receiving the wound treatment composition carrier.

6. The contactless medicine applicating apparatus as recited in claim 1, the apparatus further comprising a wound treatment barrier body viewing port enabling viewing through the volume encapsulating body.

7. The contactless medicine applicating apparatus as recited in claim 1, wherein the apparatus is of a shape following a contour of a foot of the patient, the apparatus further comprising a contactless wound treatment shoe insole located proximate a lower interior surface of the volume encapsulating body, wherein the contactless wound treatment shoe insole carries an additional volume of the wound treatment composition in at least one of the liquid state and the solid state.

8. The contactless medicine applicating apparatus as recited in claim 1, the volume encapsulating body is fabricated of at least one of: Ethylene-Vinyl Acetate Copolymer (EVA foam), Poly Ethylene-Vinyl Acetate Copolymer, foam, rubber, silicone, silicone-rubber, nylon, flexible plastic, clear flexible plastic, leather, and vinyl.

9. The contactless medicine applicating apparatus as recited in claim 1, the volume encapsulating body further comprising a plurality of small perforations passing therethrough.

10. The contactless medicine applicating apparatus as recited in claim 1, the wound treatment composition carrier further comprising a flow rate control mechanism for adjusting release of the wound treatment composition in the gaseous state, wherein the flow rate control mechanism is adjusted using at least one of a manual process and an automated process.

11. A contactless medicine applicating apparatus comprising:
a wound treatment barrier comprising a volume encapsulating body, wherein an interior surface of the volume encapsulating body defines a gas retaining volume;
a wound treatment composition carrier, the wound treatment composition carrier carrying a wound treatment composition in at least one of a solid state and a liquid state, wherein the wound treatment composition carrier has a property enabling outgassing of the wound treatment composition in a gaseous state;
a plurality of small perforations passing through the wound treatment barrier, the small perforations sized to allow an influx of Oxygen while limiting outflow of the gaseous wound treatment composition in the gaseous state;
a wound treatment composition carrier receiving aperture formed through the volume encapsulating body, the wound treatment aperture is of a shape and size to receive and retain the wound treatment composition carrier,
wherein the wound treatment composition contains iodine,
wherein in use, the wound treatment composition carrier is removably inserted into the wound treatment composition carrier receiving aperture and a gaseous state of the wound treatment composition is discharged into the gas retaining volume defined by the interior surface of the volume encapsulating body,
wherein the volume encapsulating body is of a shape and size to encapsulate a exposed wound of a patient and at least one sealing element that provides a substantially gas impervious seal against skin of a patient;
wherein the volume encapsulating body is of a shape and size to encapsulate the exposed wound of the patient while avoiding contact between the wound treatment composition carrier and the exposed wound of the patient.

12. The contactless medicine applicating apparatus as recited in claim 11, wherein the volume encapsulating body is provided in one of the following shapes:
(a) a dome shape,
(b) a dome shape having a flange extending radially outward from a peripheral edge thereof,
(c) a J-shape,
(d) a J-shape having a seal located proximate an opening at a top of the J-shape,
(e) a foot covering,
(f) a foot covering having a seal located proximate an opening at an insertion region of the foot covering, and
(g) a glove.

13. The contactless medicine applicating apparatus as recited in claim 11, the wound treatment composition carrier further comprising a mechanically assembling feature for assembling the wound treatment composition carrier within the wound treatment aperture of the volume encapsulating body.

14. The contactless medicine applicating apparatus as recited in claim 11, the apparatus further comprising a wound treatment composition container body receiving member inserted through the wound treatment composition carrier receiving aperture.

15. The contactless medicine applicating apparatus as recited in claim 14, the wound treatment composition container body receiving member further comprising threading for threadably receiving the wound treatment composition carrier.

16. The contactless medicine applicating apparatus as recited in claim 11, the apparatus further comprising a wound treatment barrier body viewing port enabling viewing through the volume encapsulating body.

17. The contactless medicine applicating apparatus as recited in claim 11, wherein the apparatus is of a shape following a contour of a foot of the patient, the apparatus further comprising a contactless wound treatment shoe insole located proximate a lower interior surface of the volume encapsulating body, wherein the contactless wound treatment shoe insole carries an additional volume of the wound treatment composition in at least one of the liquid state and the solid state.

18. The contactless medicine applicating apparatus as recited in claim 11, the volume encapsulating body is fabricated of at least one of: Ethylene-Vinyl Acetate Copolymer (EVA foam), Poly Ethylene-Vinyl Acetate Copolymer, foam, rubber, silicone, silicone-rubber, nylon, flexible plastic, clear flexible plastic, leather, and vinyl.

19. The contactless medicine applicating apparatus as recited in claim 11, the volume encapsulating body further comprising a plurality of small perforations passing therethrough.

20. The contactless medicine applicating apparatus as recited in claim 11, the wound treatment composition carrier further comprising a flow rate control mechanism for adjusting release of the wound treatment composition in the gaseous state, wherein the flow rate control mechanism is adjusted using at least one of a manual process and an automated process.
